(19) United States
(12) Patent Application Publication
(10) Pub. No.: US 2022/0040331 A1
(43) Pub. Date: Feb. 10, 2022

(54) COMPOSITIONS AND METHODS FOR TRANSFECTING CELLS

(71) Applicant: AMRYT GENETICS LTD., Dublin (IE)

(72) Inventors: Lara Cutlar, Dublin (IE); Wenxin Wang, Dublin (IE)

(21) Appl. No.: 17/284,607

(22) PCT Filed: Oct. 14, 2019
(86) PCT No.: PCT/US2019/056151

§ 371 Date: Apr. 12, 2021

Related U.S. Application Data
(60) Provisional application No. 62/826,461, filed on Mar. 29, 2019.
(60) Provisional application No. 62/744,994, filed on Oct. 12, 2018.

(51) Int. Cl. A61K 48/00; C08G 83/00; A61K 31/7105; A61K 38/46; A61K 38/39; A61K 47/64; A61K 9/19

(52) U.S. Cl. CPC A61K 48/0041; C08G 83/002; A61K 31/7105; A61K 9/19; A61K 38/39; A61K 47/641; A61K 38/465

(57) ABSTRACT

The present disclosure relates to branched polymers and polyplexes which find use in gene therapy applications as safe and non-toxic nucleic acid transfection agents.

### CROSS-REFERENCE TO RELATED APPLICATIONS

The present application claims the benefit of priority to U.S. Provisional Application No. 62/744,994, filed Oct. 12, 2018, and U.S. Provisional Application No. 62/826,461, filed Mar. 29, 2019, the contents of which are hereby incorporated by reference in their entirety.

### FIELD OF DISCLOSURE

Various embodiments of the present disclosure relate to branched polymers which find use, e.g., in gene therapy applications as safe and non-toxic nucleic acid transfection agents.

### BACKGROUND

Delivery of functional genetic materials into cell (e.g., skin fibroblast cells) to manipulate the transgene expression is of great significance in nanomedicine. Despite numerous polymeric gene delivery systems having been developed, highly safe and efficient gene transfection (e.g., fibroblast gene transfection) has not yet been achieved.

Following almost three decades of development, gene therapy has become a predominant part of the rapidly increasing armamentarium of nanomedicine for improving health conditions and correcting genetic disorders.^{[1]} Although multiple clinical trials using viral gene delivery vectors have been carried out, the risks of triggering immunogenic responses and transgene insertional mutagenesis, limitations associated with large-scale production and low “cargo capacity” for genetic materials, along with the unpredictability of vector mobility remain unaddressed.^{[2,3]} From this perspective, non-viral gene delivery vectors would be more promising because of their potential for minimal immunogenicity, non-tumorigenicity, cost-effective manufacturing, high payload of nucleic acids and localized gene expression. From 2010 onward, the number of clinical trials for gene therapies using non-viral gene vectors has increased remarkably; plasmid DNAs and small interfering RNAs (siRNA) have been formulated in at least 40 nanoparticle-based gene therapies for gene correction, therapeutic protein expression and antigen vaccination, with 12 major liposome systems investigated in 27 clinical trials and 7 polymer-based systems in 13 clinical trials.^{[3]} Among the polymer-based gene therapy clinical trials, the off-the-shelf cationic polymer polyethylenimine (PEI) has showed some promises. However, PEI is nondegradable and severely hampered by concerns about its safety.^{[4]} Therefore, tremendous efforts have been made to improve the gene transfection efficiency and safety of polymeric gene vectors so that the polymer-based gene therapies can be brought closer to clinical applications.

Among polymeric gene delivery vectors, poly(β-amino ester)s (PAEs) are one type of the most promising candidates. PAEs were first designed and synthesized by Langer and co-workers by the copolymerization of amines with diacrylates through a one-step Michael addition process.^{[5]} The tertiary amines on the backbone and primary amines at the terminals serve as the cationic units to condense DNA into nanomeric particles through electrostatic interactions, and to facilitate polyplex escape from endo/lysosomes via the “proton sponge effect”, and the ester bonds on the backbone can be hydrolytically degraded under aqueous conditions to dissociate the polyplexes and release DNA as well as reduce the cytotoxicity after gene transfection.^{[6,7]} After intensive structure/property optimization,^{[8-10]} several PAEs have been identified for DNA transfection with favorable safety profile and high transfection efficiency, both in vitro and in vivo.^{[6,11,12]} However, until 2015, almost all of the studies with PAEs had been focused on polymers with a linear structure. Branched polymers may have greater potential for gene transfection because their three-dimensional (3D) structure and multiple terminal functional groups would bestow the polymeric gene vectors with additional advantages, we have successfully developed highly branched poly(β-amino ester)s (HPAEs) via a facile one-pot “A2+B3+C2” Michael addition strategy.^{[13-16]} Over a wide range of cell types, HPAEs exhibited much higher gene transfection ability in comparison with their corresponding linear counterparts, demonstrating their greater potential in gene delivery. The high gene transfection capability of HPAEs was further demonstrated in vivo using the recessive dystrophic epidermolysis bullosa (RDEB) skin disease model. RDEB is a rare, devastating, hereditary mechanobullous disorder caused by the mutation of COL7A1 gene that encodes type VII collagen (C7), which is a key component of anchoring fibrils (AFs) that serve to secure the epidermal-dermal adherence^{[17]}. The deficiency of C7 leads to skin fragility, widespread bullae, and erosions that characteristically heal with exuberant scarring and milia formation.^{[18]} In both the RDEB knockout mouse model and grafting mouse model, HPAEs mediated high level and up to 10-week restoration of C7 expression,^{[13,15,19]} highlighting their huge potential for clinical skin gene delivery. HPAEs are further described in U.S. Patent Publication No. 2017/0216455, which is hereby incorporated by reference in its entirety for all purposes.

Fibroblasts play a pivotal role in maintaining the integrity of skin tissue and skin biological function, regulating cellular microenvironment, and are associated with multiple skin diseases such as hypertrophic scarring, aging/photoaging, diabetic wound healing, cancer, and pachydermoperiostosis. The ability to manipulate gene expression within fibroblasts is fundamental for functional genomics, pathway analysis, and biomedical applications. For example, primary human dermal fibroblasts (HPDF) are an accessible source of phenotypically and karyotypically normal human skin cells, biologically more relevant to in vivo applications in comparison with the immortalized cell lines.^{[20]} Previously, HPDF were directly injected into the skin for C7 restoration in RDEB. Nevertheless, direct intradermal injection of HPDF shows abnormal morphology of the AFs^{[21]} and transient protein replacement^{[22]} in RDEB patients. In contrast, it can be envisaged that after genetic engineering by transfection, fibroblasts can be diversely adapted and made more suitable for clinical gene therapy. C7 enhancement of the HPDF would have a significant effect on improving the strength and stability of the reconstructed AFs, optimizing the dosing schedule and reducing the administration frequency in RDEB. However, non-viral gene transfection of fibroblasts has always been challenging. The most common methods include expensive electroporation, magnetofection and relatively inefficient and toxic chemical formulations.^{[20,23,24]} For instance, only 27% and 44% of enhanced green fluorescence protein (EGFP) delivery efficiencies in human dermal fibroblast^{[25]} and human primary fibroblasts^{[26]} were detected by different electroporation systems. The maximum transfection efficiency of the leading cationic lipid reagents TransFectin, Lipofectamine LTX and electroporation in the mouse embryonic fibroblast was 15.7%, 11.8% and 48.1%, respectively^{[24]}.

Therefore, the development of a reliable non-viral gene delivery system to transfect fibroblasts with high efficiency and safety is imperative and of great significance.

### SUMMARY

In some embodiments, the present disclosure provides branched polymers suitable for forming polyplexes useful for gene transfection therapies made by a process of:

(a) reacting a compound of formula (A)

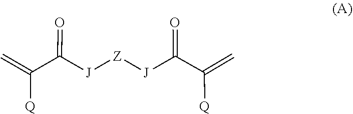

with a first amine having the formula R₁—NH₂ or R₁—N(H)—Z′—N(H)—R₁;
(b) reacting the product of Step (a) with a second amine having the formula R₂—NH₂ or R₂—N(H)—Z″—N(H)—R₂; and
(c) reacting the product of Step (b) with a compound of formula (B):

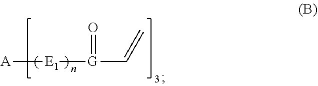

wherein

each J is independently —O— or —NH—;

Z, Z′, and Z″ are linking moieties;

A is a linear or branched carbon chain of 1 to 30 carbon atoms, a linear or branched heteroatom-containing carbon chains of 2 to 30 atoms, a carbocycle containing 3 to 30 carbon atoms, or a heterocycle containing 3 to 30 atoms;

wherein A is optionally substituted with one or more halogen, hydroxyl, amino group, sulfonyl group, sulphonamide group, thiol, C₁-C₆ alkyl, C₁-C₆ alkoxy, C₁-C₆ether, C₁-C₆ thioether, C₁-C₆ sulfone, C₁-C₆ sulfoxide, C₁-C₆ primary amide, C₁-C₆ secondary amide, halo C₁-C₆ alkyl, carboxyl group, cyano group, nitro group, nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl or C₆-C₁₀ aryl; wherein each R′ is independently selected, from the group consisting of hydrogen and C₁-C₆ alkyl;

G is —C—, —S—, —S(O)—, —P(OR₁)—, or —P(OH)—;

each Q is a C₁-C₁₀ linear or branched alkyl group;

each E₁ is independently selected from the group consisting of covalent bond, —N—, —O—, —S—, alkylene, heteroalkylene, alkenyl, heteroalkenylene, alkynyl, heteroalkynylene;

R₁ and R₂ are each independently C₁-C₄₀alkyl, C₁-C₄₀ heteroalkyl, C₂-C₄₀alkenyl, C₂-C₄₀ heteroalkenylene, C₄-C₈cycloalkenyl, C₂-C₄₀alkynyl, C₂-C₄₀ heteroalkynylene, C₃-C₈cycloalkyl, heterocyclyl, aryl, or heteroaryl; wherein the heterocyclyl and heteroaryl contain 1-5 heteroatoms selected from the group consisting of N, S, P and O; wherein the C₁-C₄₀alkyl, C₂-C₄₀alkenyl, C₄-C₈cycloalkenyl, C₂-C₄₀alkynyl, C₃-C₈cycloalkyl, heterocyclyl, aryl, and heteroaryl are optionally substituted with D, halogen, C₁-C₆alkyl, —OH, —O—C₁-C₆alkyl, —NH₂, —NH(C₁-C₆alkyl), or —N(C₁-C₆alkyl)₂; and R₁ is unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ ether, a C₁-C₆ thioether, a C₁-C₆ sulfone, a C₁-C₆ sulfoxide, a C₁-C₆ primary amide, a C₁-C₆ secondary amide, a halo C₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl and C₆-C₁₀) aryl; wherein each R′ is independently selected, from the group consisting of hydrogen and C₁-C₆ alkyl; and

each n is at least 1.

In some embodiments, the present disclosure provides a method of making a polymer comprising:

(a) reacting a compound of formula (A)

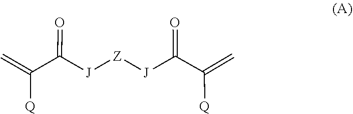

with a first amine having the formula R₁—NH₂ or R₁—N(H)—Z′—N(H)—R₁;
(b) reacting the product of (a) with a second amine having the formula R₂—NH₂ or R₁—N(H)—Z″—N(H)—R₂; and
(b) reacting the product of (b) with a compound of formula (B):

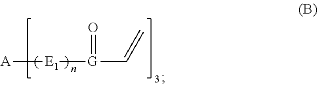

wherein

each J is independently —O— or —NH—;

Z, Z′, and Z″ are linking moieties;

A is a linear or branched carbon chain of 1 to 30 carbon atoms, a linear or branched heteroatom-containing carbon chains of 2 to 30 atoms, a carbocycle containing 3 to 30 carbon atoms, or a heterocycle containing 3 to 30 atoms;

wherein A is optionally substituted with one or more halogen, hydroxyl, amino group, sulfonyl group, sulphonamide group, thiol, C₁-C₆ alkyl, C₁-C₆ alkoxy, C₁-C₆ether, C₁-C₆ thioether, C₁-C₆ sulfone, C₁-C₆ sulfoxide, C₁-C₆ primary amide, C₁-C₆ secondary amide, halo C₁-C₆ alkyl, carboxyl group, cyano group, nitro group, nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl or C₆-C₁₀ aryl; wherein each R′ is independently selected, from the group consisting of hydrogen and C₁-C₆ alkyl;

G is —C—, —S—, —S(O)—, —P(OR₁)—, or —P(OH)—;

each Q is H or a C₁-C₁₀ linear or branched alkyl group;

each E₁ is independently selected from the group consisting of covalent bond, —N—, —O—, —S—, alkylene, heteroalkylene, alkenyl, heteroalkenylene, alkynyl, heteroalkynylene;

R₁ and R2 are each independently C₁-C₄₀alkyl, heteroalkyl, C₂-C₄₀alkenyl, C₂-C₄₀ heteroalkenylene, C₄-C₈cycloalkenyl, C₂-C₄₀alkynyl, C₂-C₄₀ heteroalkynylene, C₃-C₈cycloalkyl, heterocyclyl, aryl, or heteroaryl; wherein the heterocyclyl and heteroaryl contain 1-5 heteroatoms selected from the group consisting of N, S, P and O; wherein the C₁-C₄₀alkyl, C₂-C₄₀alkenyl, C₄-C₈cycloalkenyl, C₂-C₄₀alkynyl, C₃-C₈cycloalkyl, heterocyclyl, aryl, and heteroaryl are optionally substituted with D, halogen, C₁-C₆alkyl, —OH, —NH(C₁-C₆alkyl), or —N(C₁-C₆alkyl)₂; and R₁ is unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ ether, a C₁-C₆ thioether, a C₁-C₆ sulfone, a C₁-C₆ sulfoxide, a C₁-C₆ primary amide, a C₁-C₆ secondary amide, a halo C₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl and C₆-C₁₀) aryl; wherein each R′ is independently selected, from the group consisting of hydrogen and C₁-C₆ alkyl; and

each n is at least 1.

In some embodiments, the present disclosure provides a polyplex comprising a nucleic acid component and either a polymer prepared by the processes described herein or a polymer comprising formula (I)

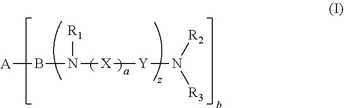

wherein

each A is independently a linear or branched carbon chain of 1 to 30 carbon atoms, a linear or branched heteroatom-containing carbon chains of 1 to 30 atoms, a carbocycle containing 3 to 30 carbon atoms, or a heterocycle containing 3 to 30 atoms;

wherein A is optionally substituted with one or more halogen, hydroxyl, amino group, sulfonyl group, sulphonamide group, thiol, C₁-C₆ alkyl, C₁-C₆ alkoxy, C₁-C₆ether, C₁-C₆ thioether, C₁-C₆ sulfone, C₁-C₆ sulfoxide, C₁-C₆primary amide, C₁-C₆ secondary amide, halo C₁-C₆ alkyl, carboxyl group, cyano group, nitro group, nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl or C₆-C₁₀ aryl; wherein each R′ is independently selected, from the group consisting of hydrogen and C₁-C₆ alkyl;

each B is independently a first linking moiety;

each X is independently

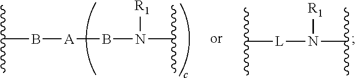

each Y is independently

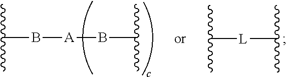

each L is independently a second linking moiety;

each R₁, R₂ and R₃ are independently, at each occurrence H, C₁-C₄₀alkyl, C₁-C₄₀ heteroalkyl, C₂-C₄₀alkenyl, C₂-C₄₀ heteroalkenylene, C₄-C₈cycloalkenyl, C₂-C₄₀alkynyl, C₂-C₄₀ heteroalkynylene, C₃-C₈cycloalkyl, heterocyclyl, aryl, or heteroaryl; wherein the heterocyclyl and heteroaryl contain 1-5 heteroatoms selected from the group consisting of N, S, P and O; wherein the C₁-C₆alkyl, C₂-C₈alkenyl, C₄-C₈cycloalkenyl, C₂-C₆alkynyl, C₃-C₈cycloalkyl, heterocyclyl, aryl, and heteroaryl are optionally substituted with D, halogen, C₁-C₆alkyl, —OH, —O—C₁-C₆alkyl, —NH₂, —NH(C₁-C₆alkyl), or —N(C₁-C₆alkyl)₂; or

wherein R₂ and R₃ together with the atom to which they are attached can form heterocyclyl or heteroaryl containing 1-3 heteroatoms selected from the group consisting of N, S, P and O;

a is 1-1000;

b is 1-4;

c is 1-3; and

z is 1-100;

with the proviso that at least one of R₂ and R₃ is not H.

In some embodiments, the present disclosure provides a pharmaceutical composition comprising an effective amount of a polyplex in accordance with certain embodiments of present disclosure, in combination with a pharmaceutically acceptable carrier.

In some embodiments, the present disclosure provides a method of cell transfection comprising contacting one or more target cells with a pharmaceutical composition comprising at least one polyplex in accordance with certain embodiments of the present disclosure, under conditions suitable to transfect the target cell with the one or more polyplexes.

In some embodiments, the present disclosure provides a method of treating a disease in a patient in need thereof, comprising administering a therapeutically effective amount of the pharmaceutical composition comprising at least one polyplex in accordance with certain embodiments of the present disclosure, such that one or more of the patient's cells are transfected with the polyplex nucleic acid component.

In some embodiments, the present disclosure provides a method of treating a disease in a patient in need thereof, comprising administering a therapeutically effective amount of the pharmaceutical composition comprising at least one polyplex in accordance with certain embodiments of the present disclosure, wherein the administration of the composition corrects a defective translation of a target gene in the subject.

### BRIEF DESCRIPTION OF THE DRAWINGS

FIG. 1 shows transfection efficiency and cell viability assessment. FIG. 1a shows Gluc activity and cell viability of HPDF 48 h post transfection by the LBPAE/DNA, PEI/DNA and SuperFect/DNA polyplexes at a series of w/w ratios. FIG. 1b shows Gluc activity and cell viability of 3T3. Significant difference from the *PEI and ^{#}SuperFect group in Gluc activity (p<0.05, Student's two-tailed t test).

FIG. 2 shows LC₅₀ assessment of LBPAE/DNA polyplexes and SuperFect/DNA polyplexes in HPDF and 3T3. FIG. 2a shows representative live/dead images of the untreated cells or cells transfected with the LBPAE/DNA polyplexes at the concentration of 555 μg mL⁻¹ or SuperFect/DNA polyplexes at the concentration of 35 μg mL⁻¹. The scale bars are 50 μm. FIG. 2b shows LBPAE/DNA polyplex concentration-dependent cell viability measured by Alamarblue assay. FIG. 2c shows SuperFect/DNA polyplex concentration-dependent cell viability measured by Alamarblue assay.

FIG. 3 shows a comparison of GFP expression and MFI mediated by different gene delivery systems. FIG. 3a shows GFP images of HPDF cells after the treatment with LBPAE/DNA, PEI/DNA and SuperFect/DNA polyplexes. Untreated (UT) cells were used as the negative control. Scale bar, 200 μm. FIG. 3b shows histogram distribution of HPDF populations after transfection with different polyplexes. FIG. 3c shows percentage of GFP positive HPDF and the MFI of cells after transfection. FIG. 3d shows GFP images of 3T3. Scale bar, 200 μm. FIG. 3e shows Histogram distribution of 3T3 populations after transfection with different polyplexes. FIG. 3f shows percentage of GFP positive 3T3 and the MFI of cells after transfection. Significant difference from commercial reagent groups in the *percentage of GFP positive cells and ^{#}MFI (p<0.05, Student's two-tailed t test).

FIG. 4 shows physicochemical characteristics of the LBPAE/DNA polyplexes. FIG. 4a shows DNA condensation ability determination with agarose gel electrophoresis. FIG. 4b shows DNA binding affinity measurement with PicoGreen assay. FIG. 4c shows polyplex size and zeta potential measurements. FIG. 4d shows polyplex morphology observation with TEM. Scale bar, 200 nm.

FIG. 5 shows cellular uptake of diverse polyplexes. FIG. 5a shows fluorescent images of cells 4 hours post transfections with different polyplexes. The nucleus was stained with DAPI (blue), DNA was labeled with Cy3 (red). Scale bar, 20 μm. FIG. 5b shows polyplex uptake efficiency in HPDF quantified with flow cytometry. FIG. 5c shows polyplex uptake efficiency in 3T3 quantified with flow cytometry. FIG. 5d shows percentage of Cy3 positive HPDF and the normalized MFI of cells. FIG. 5e shows percentage of Cy3 positive 3T3 and the normalized MFI of cells. Significant difference from SuperFect in the * MFI quantification (p<0.05, Student's two-tailed t test).

FIG. 6 shows proton buffering capacity, degradation and DNA release assessment of the LBPAE. FIG. 6a shows proton buffering capacity determined by acid-base titration. FIG. 6b shows degradation profile determined using GPC. FIG. 6c shows evaluation of DNA release from polyplexes assessed with PicoGreen assay.

FIG. 7 shows immunofluorescence staining of C7 expression in the HPDF. FIG. 7a shows fluorescent images of the HPDF four days post transfection with the LBPAE/MCC7 polyplexes. The nucleus was stained with DAPI (blue) and the C7 was incubated with monoclonal anti-collagen VII primary antibody and stained with Alexa-568 goat anti-mouse secondary antibody (red). Scale bar, 20 μm. FIG. 7b shows flow cytometry quantification of C7 expression of HPDF. FIG. 7c shows degree of C7 expression upregulation and the MFI of HPDF after transfection with LBPAE/MCC7 and SuperFect/DNA polyplexes. Significant difference from SuperFect in the *percentage of C7 upregulation and ^{#}MFI (p<0.05, Student's two-tailed t test).

FIG. 8 is a schematic illustration of the synthesis of LBPAE through the linear oligomer combination strategy. In step 1, A2 type amine reacts with C2 type diacrylate to form the linear A2-C2 base oligomer, which is further end-capped by a second amine to generate the linear A2-C2 oligomer. In Step 2, the linear A2-C2 oligomer is combined by the B3 type triacrylate by branching to yield LBPAE. The box shows the monomers and end-capping agent used for the synthesis of LBPAE in this work.

FIG. 9 shows GPC results of linear oligomer and LBPAE.

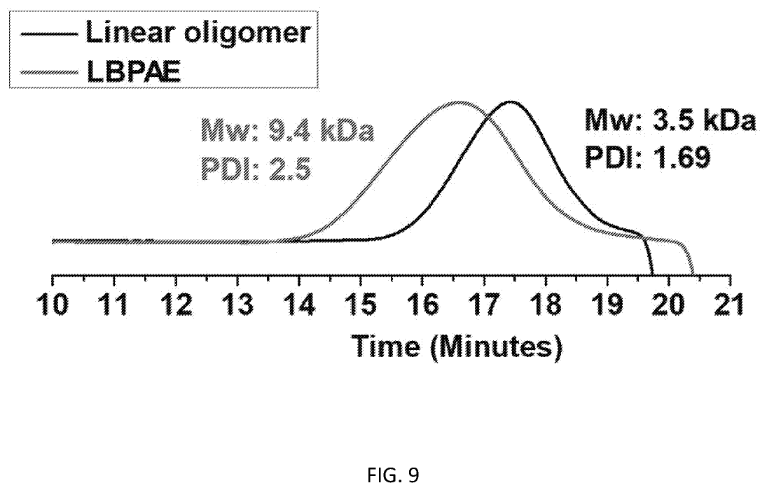

FIG. 10 shows MH Alpha curve and value of LBPAE.

FIG. 11 shows chemical composition analysis of LBPAE by ¹H NMR.

FIG. 12 shows agarose gel results of MCC7 and pcDNA3.1COL7A1.

FIG. 13a shows the HPAE synthesis via the “A2+B3+C2” Michael addition strategy. FIG. 13b shows GPC curves and calculated Mw of HPAEs of the present disclosure. FIG. 13c shows MH Alpha curves and calculated values of HPAEs of the present disclosure. (The FIGS. 13 and 16-19 use “HC32-122” to define the HPAE polymer of the present disclosure. HC32-122 is equivalent to HC32-DATOU, which is used in the Examples.)

FIG. 14 shows transfection of RDEB keratinocytes using polyplexes comprising HPAEs having MW 11 kDa, 21 kDa, 34 kDa and 41 kDa using HPAE/DNA ratios of 10:1, 30:1 and 50:1 (weight %/weight %).

FIG. 15 shows cell viability test after gene transfection of RDEB keratinocytes using polyplexes comprising HPAEs having MW 11 kDa, 21 kDa, 34 kDa and 41 kDa using polymer/DNA ratios of 10:1, 30:1 and 50:1 (weight %/weight %).

FIG. 16 shows reporter gene transfection studies in RDEBK cells using HPAE/DNA polyplexes. FIG. 16a shows relative Gluc activity of RDEBK cells 48 h post transfection by HPAE/DNA and PEI/DNA polyplexes. Data presented as the percentage normalized to the Gluc activity of RDEBK cells transfected by HPAE/DNA polyplexes (30:1 wt %/wt %). *Significant difference from the HPAE group (w/w=30:1) (p<0.05, Student's t-test); FIG. 16b shows viability of RDEBK cells after transfection with HPAE/DNA and PEI/DNA polyplexes; FIG. 16c shows GFP images of untreated (UT) cells, cells treated with HPAE/DNA (w/w=30:1) or PEI/DNA (w/w=1:1) polyplexes. Scale bar, 200 μm; FIG. 16d shows representative histogram distributions of UT and transfected cell population; FIG. 16e shows percentage of GFP-positive RDEBK cells and MFI quantified with flow cytometry. Significant difference from PEI in the *percentage of GFP-positive cells and ^{#}cell MFI (p<0.05, Student's t-test).

FIG. 17 shows MCC7 biosynthesis and cellular uptake of HPAE/MCC7 polyplexes. FIG. 17a shows the MCC7 biosynthesis with phiC31 plus 1-scel digest system. FIG. 17b shows agarose gel electrophoresis of three COL7A1-encoding plasmid DNA after EcoR1 digestion. Regular plasmid (RP) of pcDNA3.1COL7A1, parental plasmid (PP) of MN511A-1-COL7A1 and MCC7 have 5 kb, 8 kb and 3 kb backbone lengths, respectively; FIG. 17c shows fluorescent images of RDEBK cells after transfection with different polyplexes. The nucleus was stained with DAPI (blue), DNA was labeled with Cy3 (red). Scale bar, 20 μm; FIG. 17d shows polyplex cellular uptake efficiency quantified with flow cytometry; FIG. 17e shows the percentage of Cy3-positive cells and MFI. *Significant difference from the PEI/MCC7 group in cell MFI (p<0.05, Student's t-test).

FIG. 18 shows COL7A1 mRNA and recombinant C7 expression following transfection with HPAE/MCC7 polyplexes. FIG. 18a shows amplification plot of endogenous control GAPDH obtained by RT-qPCR; FIG. 18b shows amplification plot of COL7A1 mRNA of RDEBK cells after transfection obtained by RT-qPCR; FIG. 18c shows COL7A1 mRNA quantification, * Significant difference from PEI group (p<0.05, Student's t-test); FIG. 18d shows Cyto-immunofluorescence images of C7 staining (red fluorescence), scale bar, 20 μm; FIG. 18e shows Western blotting results of C7 expression. The 42-kDa β-Actin was used as the loading control.

FIG. 19 shows physicochemical properties of HPAE/MCC7 polyplexes at the HPAE/DNA wt %/wt % ratio of 30:1. FIG. 19a shows HPAE/MCC7 polyplex formation; FIG. 19b shows agarose gel results of DNA condensation and heparin competition assay 2 h post polyplex preparation; FIG. 19c shows DNA binding ability test by PicoGreen assay with or without the presence of heparin 2 h post polyplex preparation; FIG. 19d shows the size of HPAE/MCC7 polyplexes measured by NTA; FIG. 19e shows the Zeta potential distribution of HPAE/MCC7 polyplexes; FIG. 19f shows the TEM image of HPAE/MCC7 polyplexes. Scale bar, 500 nm.

FIG. 20 shows gene transfection performance of a formulations comprising a HPAE polyplex of the present disclosure. FIG. 20a shows polyplex lyophilization and further transfection studies in RDEBK cells; FIG. 20b shows GFP images of cells after transfection with polyplexes from different storage methods and lyophilization conditions. FZ: freeze-drying; Suc: sucrose. Scale bar, 200 μm; FIG. 20c shows Representative histogram distributions of UT and transfected cell population; FIG. 20d shows GFP expression efficiency of cells after transfection quantified by flow cytometry. *Significant difference from the freshly prepared polyplex group (p<0.05, Student's t-test); (e) Normalized MFI quantified by flow cytometry. *Significant difference from the freshly prepared polyplex group (p<0.05, Student's t-test).

FIG. 21 shows the transfection of HPAE polyplexes of the present disclosure into RDEBK cells following long-term storage

### DETAILED DESCRIPTION

As used above, and throughout this disclosure, the following terms, unless otherwise indicated, shall be understood to have the following meanings. If a term is missing, the conventional term as known to one skilled in the art controls.

As used herein, the terms “including,” “containing,” and “comprising” are used in their open, non-limiting sense.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

A “patient” is a mammal, e.g., a human, mouse, rat, guinea pig, dog, cat, horse, cow, pig, or non-human primate, such as a monkey, chimpanzee, baboon or rhesus monkey. “Patient” includes both humans and animals.

The terms “effective amount” or “therapeutically effective amount” when used in connection with a compound refer to a sufficient amount of the compound to provide the desired biological result. That result can be reduction and/or alleviation of the signs, symptoms, or causes of a disease, or any other desired alteration of a biological system. For example, an “effective amount” for therapeutic use is the amount of the composition comprising a compound as disclosed herein required to provide a clinically significant decrease in a disease. An appropriate “effective amount” in any individual case may be determined by one of ordinary skill in the art using routine experimentation. Thus, the expression “effective amount” generally refers to the quantity for which the active substance has therapeutic effects.

As used herein, the terms “treat” or “treatment” are synonymous with the term “prevent” and are meant to indicate a postponement of development of diseases, preventing the development of diseases, and/or reducing severity of such symptoms that will or are expected to develop. Thus, these terms include ameliorating existing disease symptoms, preventing additional symptoms, ameliorating or preventing the underlying causes of symptoms, inhibiting the disorder or disease, e.g., arresting the development of the disorder or disease, relieving the disorder or disease, causing regression of the disorder or disease, relieving a condition caused by the disease or disorder, or stopping or alleviating the symptoms of the disease or disorder. In certain embodiments, “treat” or “treatment” refers to promoting a healthy skin phenotype.

The term “disorder” is used in this disclosure to mean, and is used interchangeably with, the terms disease, condition, or illness, unless otherwise indicated.

By using the terms “pharmaceutically acceptable” or “pharmacologically acceptable” it is intended to mean a material which is not biologically, or otherwise, undesirable—the material may be administered to an individual without causing any substantially undesirable biological effects or interacting in a deleterious manner with any of the components of the composition in which it is contained.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject. Excipients should be selected on the basis of compatibility and the release profile properties of the desired dosage form. Exemplary carrier materials include, e.g., binders, suspending agents, disintegration agents, filling agents, surfactants, solubilizers, stabilizers, lubricants, wetting agents, diluents, spray-dried dispersions, and the like.

The term “pharmaceutically compatible carrier materials” may comprise, e.g., acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, and the like. See, e.g., Hoover, John E., Remington's Pharmaceutical Sciences, Mack Publishing Co., Easton, Pa. 1975.

As used herein, the term “subject” encompasses mammals and non-mammals. Examples of mammals include, but are not limited to, any member of the class Mammalia: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the present disclosure, the mammal is a human.

The terms “administered”, “administration”, or “administering” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body, including an animal, in need of treatment by bringing such individual in contact with, or otherwise exposing such individual to, such compound.

As used herein, “alkyl” means a straight chain or branched saturated chain having from 1 to 40 carbon atoms. Representative saturated alkyl groups include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, n-pentyl, isopentyl, neopentyl, n-hexyl and the like, and longer alkyl groups, such as heptyl, and octyl and the like. An alkyl group can be unsubstituted or substituted. Alkyl groups containing three or more carbon atoms may be straight, or branched. As used herein, “lower alkyl” means an alkyl having from 1 to 10 carbon atoms.

As used herein, an “alkenyl” includes an unbranched or branched hydrocarbon chain containing 2-40 carbon atoms. The “alkenyl” group contains at least one double bond. The double bond of an alkenyl group can be unconjugated or conjugated to another unsaturated group. Examples of alkenyl groups include, but are not limited to, ethylenyl, vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl and the like. An alkenyl group can be unsubstituted or substituted. Alkenyl, as defined herein, may also be branched or straight.

As used herein, “alkynyl” includes an unbranched or branched unsaturated hydrocarbon chain containing 2-40 carbon atoms. The “alkynyl” group contains at least one triple bond. The triple bond of an alkynyl group can be unconjugated or conjugated to another unsaturated group. Examples of alkynyl groups include, but are not limited to, ethynyl, propynyl, butynyl, pentynyl, hexynyl, methylpropynyl, 4-methyl-1-butynyl, 4-propyl-2-pentynyl, 4-butyl-2-hexynyl and the like. An alkynyl group can be unsubstituted or substituted.

It should also be noted that any carbon as well as heteroatom with unsatisfied valences in the text, schemes, examples and Tables herein is assumed to have the sufficient number of hydrogen atom(s) to satisfy the valences.

As used herein, references to hydrogen may also refer to a deuterium substitution if desired. The term “deuterium” as used herein means a stable isotope of hydrogen having odd numbers of protons and neutrons.

The term “halo” or “halogen” refers to fluorine, chlorine, bromine, or iodine.

The term “haloalkyl” as used herein refers to an alkyl group, as defined herein, which is substituted one or more halogen. Examples of haloalkyl groups include, but are not limited to, trifluoromethyl, difluoromethyl, pentafluoroethyl, trichloromethyl, etc.

Unless otherwise specifically defined, the term “aryl” refers to cyclic, aromatic hydrocarbon groups that have 1 to 3 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. The substituents can themselves be optionally substituted. Furthermore when containing two fused rings the aryl groups herein defined may have an unsaturated or partially saturated ring fused with a fully saturated ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic or polycyclic aromatic radical of 5 to 18 ring atoms or a polycyclic aromatic radical, containing one or more ring heteroatoms selected from N, O, or S, the remaining ring atoms being C. Heteroaryl as herein defined also means a bicyclic heteroaromatic group wherein the heteroatom is selected from N, O, or S. The aromatic radical is optionally substituted independently with one or more substituents described herein. The substituents can themselves be optionally substituted. Examples include, but are not limited to, benzothiophene, furyl, thienyl, pyrrolyl, pyridyl, pyrazinyl, pyrazolyl, pyridazinyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, benzoimidazolyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, dihydrobenzoxanyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydropyrrolo[1,2-a]pyrimidinyl, 3,4-dihydro-2H-1λ²-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore when containing two fused rings the heteroaryl groups herein defined may have an unsaturated or partially saturated ring fused with a fully saturated ring.

As used herein, the term “cycloalkyl” refers to a saturated or partially saturated, monocyclic, fused or spiro polycyclic, carbocycle having from 3 to 18 carbon atoms per ring. The cycloalkyl ring or carbocycle may be optionally substituted by one or more substituents, e.g., 1 to 5 substituents, at any point of attachment. The substituents can themselves be optionally substituted. Examples of cycloalkyl groups include, without limitations, cyclopropyl, cyclobutyl, cyclopentyl, cyclohexyl, cycloheptanyl, cyclooctanyl, norboranyl, norborenyl, bicyclo[2.2.2]octanyl, bicyclo[2.2.2]octenyl, decahydronaphthalenyl, octahydro-1H-indenyl, cyclopentenyl, cyclohexenyl, cyclohexa-1,4-dienyl, cyclohexa-1,3-dienyl, 1,2,3,4-tetrahydronaphthalenyl, octahydropentalenyl, 3a,4,5,6,7,7a-hexahydro-1H-indenyl, 1,2,3,3a-tetrahydropentalenyl, bicyclo[3.1.0]hexanyl, bicyclo[2.1.0]pentanyl, spiro[3.3]heptanyl, bicyclo[2.2.1]heptanyl, bicyclo[2.2.1]hept-2-enyl, bicyclo[2.2.2]octanyl, 6-methylbicyclo[3.1.1]heptanyl, 2,6,6-trimethylbicyclo[3.1.1]heptanyl, and derivatives thereof.

As used herein, the term “cycloalkenyl” refers to a partially saturated, monocyclic, fused or spiro polycyclic, carbocycle having from 3 to 18 carbon atoms per ring and contains at least one double bond. The cycloalkenyl ring may be optionally substituted by one or more substituents, e.g., 1 to 5 substituents, at any point of attachment. The substituents can themselves be optionally substituted.

As used herein, the term “heterocycloalkyl” or “heterocyclyl” refers to a saturated or partially unsaturated and non-aromatic monocyclic, or fused or spiro, polycyclic, ring structure of 4-to-18 atoms containing carbon and heteroatoms taken from oxygen, nitrogen, or sulfur and wherein there is not delocalized π-electrons (aromaticity) shared among the ring carbon or heteroatoms. The heterocycloalkyl or heterocyclyl ring structure may be substituted by one or more substituents. The substituents can themselves be optionally substituted. Examples of heterocycloalkyl or heterocyclyl rings include, but are not limited to, oxetanyl, azetidinyl, tetrahydrofuranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, homotropanyl, dihydrothiophen-2(3H)-onyl, tetrahydrothiophene 1,1-dioxide, 2,5-dihydro-1H-pyrrolyl, imidazolidin-2-one, pyrrolidin-2-one, dihydrofuran-2(3H)-one, 1,3-dioxolan-2-one, isothiazolidine 1,1-dioxide, 4,5-dihydro-1H-imidazolyl, 4,5-dihydrooxazolyl, oxiranyl, pyrazolidinyl, 4H-1,4-thiazinyl, thiomorpholinyl, 1,2,3,4-tetrahydropyridinyl, 1,2,3,4-tetrahydropyrazinyl, 1,3-oxazinan-2-one, tetrahydro-2H-thiopyran 1,1-dioxide, 7-oxabicyclo[2.2.1]heptanyl, 1,2-thiazepane 1,1-dioxide, octahydro-2H-quinolizinyl, 1,3-diazabicyclo[2.2.2]octanyl, 2,3-dihydrobenzo[b][1,4]dioxine, 3-azabicyclo[3.2.1]octanyl, 8-azaspiro[4.5]decane, 8-oxa-3-azabicyclo[3.2.1]octanyl, 2-azabicyclo[2.2.1]heptane, 2,8-diazaspiro[5.5]undecanyl, 2-azaspiro[5.5]undecanyl, 3-azaspiro[5.5]undecanyl, decahydroisoquinolinyl, 1-oxa-8-azaspiro[4.5]decanyl, 8-azabicyclo[3.2.1]octanyl, 1,4′-bipiperidinyl, azepanyl, 8-oxa-3-azabicyclo[3.2.1]octanyl, 3,4-dihydro-2H-benzo[b][1,4]oxazinyl, 5,6,7,8-tetrahydroimidazo[1,2-a]pyridinyl, 1,4-diazepanyl, phenoxathiinyl, benzo[d][1,3]dioxolyl, 2,3-dihydrobenzofuranyl, 2,3-dihydrobenzo[b][1,4]dioxinyl, 4-(piperidin-4-yl)morpholinyl, 3-azaspiro[5.5]undecanyl, decahydroquinolinyl, piperazin-2-one, 1-(pyrrolidin-2-ylmethyl)pyrrolidinyl, 1,3′-bipyrrolidinyl, and 6,7,8,9-tetrahydro-1H,5H-pyrazolo[1,2-a][1,2]diazepinyl.

Numerical ranges, as used herein, are intended to include sequential integers, unless otherwise noted. For example, a range expressed as “from 0 to 5” would include 0, 1, 2, 3, 4 and 5.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e., a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus the term “optionally substituted” means that a given chemical moiety has the potential to contain other functional groups, but does not necessarily have any further functional groups. If not specified otherwise, suitable substituents used in the optional substitution of the described groups include, without limitation, oxo, -halogen, C₁-C₆ alkyl, C₁-C₆ alkoxy, C₁-C₆ haloalkyl, C₁-C₆ haloalkoxy, —OC₁-C₆ alkenyl, —OC₁-C₆ alkynyl, —C₁-C₆ alkenyl, —C₁-C₆ alkynyl, —OH, CN (cyano), —CH₂CN, —OP(O)(OH)₂, —C(O)OH, —OC(O)C₁-C₆ alkyl, —C(O)C₁-C₆ alkyl, —C(O)—C₀-C₆ alkylenyl-cycloalkyl, —C(O)—C₀-C₆ alkylenyl-heterocycloalkyl, —C(O)—C₀-C₆ alkylenyl-aryl, —C(O)—C₀-C₆ alkylenyl-heteroaryl, —OC(O)OC₁-C₆ alkyl, NH₂, NH(C₁-C₆ alkyl), N(C₁-C₆ alkyl)₂, —C(O)NH₂, —C(O)NH(C₁-C₆ alkyl), —C(O)N(C₁-C₆ alkyl)₂, —C(O)NH cycloalkyl, —C(O)N(C₁-C₆alkyl)cycloalkyl, —C(O)NHheterocycloalkyl, —C(O)N(C₁-C₆ alkyl)heterocycloalkyl, —C(O)NHaryl, —C(O)N(C₁-C₆ alkyl)aryl, —C(O)NHheteroaryl, —C(O)N(C₁-C₆alkyl)heteroaryl, —S(O)₂—C₁-C₆ alkyl, —S(O)₂—C₁-C₆ haloalkyl, —S(O)₂-cycloalkyl, —S(O)₂-heterocycloalkyl, —S(O)₂-aryl, —S(O)₂-heteroaryl —C₀-C₆ alkylenyl-S(O)₂NH₂, —S(O)₂NHC₁-C₆ alkyl, —S(O)₂N(C₁-C₆alkyl)₂, —S(O)₂NHcycloalkyl, —S(O)₂NHheterocycloalkyl, —S(O)₂NHaryl, —S(O)₂NHhetereoaryl, —NHS(O)₂C₁-C₆ alkyl, —N(C₁-C₆ alkyl)S(O)₂(C₁-C₆ alkyl), —NHS(O)₂aryl, —N(C₁-C₆ alkyl)S(O)₂ aryl, —NHS(O)₂ heteroaryl, —N(C₁-C₆ alkyl)S(O)₂ heteroaryl, —NHS(O)₂ cycloalkyl, —N(C₁-C₆ alkyl)S(O)₂ cycloalkyl, —NHS(O)₂ heterocycloalkyl, —N(C₁-C₆ alkyl)S(O)₂ heterocycloalkyl, —N(C₁-C₆ alkyl)S(O)₂ aryl, —C₀-C₆ alkylenyl-aryl, —C₀-C₆ alkylenyl-heteroaryl, —C₀-C₆ alkylenyl-cycloalkyl, —C₀-C₆ alkylenyl-heterocycloalkyl, —O-aryl, —NH-aryl, and N(C₁-C₆alkyl)aryl. The substituents can themselves be optionally substituted. When a multifunctional moiety is shown, the point of attachment to the core is indicated by a line, e.g., (cycloalkyloxy)alkyl-refers to alkyl being the point of attachment to the core while cycloalkyl is attached to alkyl via the oxy group. “Optionally substituted” also refers to “substituted” or “unsubstituted”, with the meanings described above.

As used herein, the term “linker” or “linking moiety” refers to a group that connects two groups and has a backbone of between 0 and 100 atoms. A linker or linkage may be a covalent bond (i.e., backbone of 0 atoms) that connects two groups or a chain of between 1 and 100 atoms in length, for example of about 1, 2, 3, 4, 5, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100 carbon atoms in length, where the linker may be linear, branched, cyclic or a single atom. In certain cases, one or more carbon atoms of a linker backbone may be optionally substituted with a sulfur, nitrogen or oxygen heteroatom. The bonds between backbone atoms may be saturated or unsaturated. The linker may include one or more substituent groups, for example an alkyl, aryl or alkenyl group. A linker may include, without limitations, oligo(ethylene glycol), ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable.

Unless otherwise stated, the term molecular weight refers to weight average molecular weight (M_{W}).

The term “heteroalkylene” refers to a divalent alkylene having one or more carbon atoms replaced with a sulfur, oxygen, or NR^{d} where R^{d} is hydrogen or alkyl. The heteroalkylene can be linear, branched, cyclic, or combinations thereof

The term “heteroalkenylene” refers to divalent straight or branched chain hydrocarbyl groups having at least one carbon-carbon double bond, and one or more heteroatoms (e.g., N, S or O) in the backbone thereof.

The term “heteroalkynylene” refers to divalent straight or branched chain hydrocarbyl groups having at least one carbon-carbon triple bond, and one or more heteroatoms (e.g., N, S or O) in the backbone thereof.

The term “polyplex” as used herein refers to a complex between a nucleic acid and a polymer. The nucleic acid is bound to the polymer via non-covalent bonds, in particular electrostatic bonds.

The term “plasmid” refers to an extra-chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. As used herein, the term “plasmid” refers to a construct made up of genetic material (i.e., nucleic acids). Typically a plasmid contains an origin of replication which is functional in bacterial host cells, e.g., Escherichia coli, and selectable markers for detecting bacterial host cells comprising the plasmid.

The term “nanoplasmid” refers to a circular DNA sequence having a reduced bacterial sequence which provides a smaller plasmid with the desired gene insert. For example, nanoplasmids produced by an antibiotic free RNA-OUT selection system and methods of making the same are described in U.S. Pat. No. 9,109,012, which are hereby incorporated by reference in their entirety patented by Nature Technology.

The term “nucleic acid” refers to a biological polymer of nucleotide bases, and may include but is not limited to deoxyribonucleic acid (DNA), ribonucleic acid (RNA), micro RNA (miRNA), and peptide nucleic acid (PNA).

The term “minicircle” refers to small, minimally sized circular DNA derived from a parental plasmid by intramolecular recombination to remove bacterial replication sequences.

The term “gene editing system” refers to a system capable of altering a target nucleic acid by one of many DNA repair pathways.

The present disclosure is directed to a new class of branched polymers, including polymers synthesized by a linear oligomer combination strategy. In some embodiments, linear poly(β-amino ester) oligomers are connected by branching units to form multifunctional linear-branched hybrid poly(β-amino ester) (LBPAE). The polymers of the present disclosure are designed and prepared for a variety of applications, including but not limited to high-performance fibroblast gene transfection. In human primary dermal fibroblasts (HPDF) and mouse embryo fibroblasts (3T3), ultra-high transgene expression is achieved by LBPAE: up to 3292-fold enhancement in Gluciferase (Gluc) expression and nearly 100% of green fluorescence protein (GFP) expression are detected. In-depth mechanistic studies reveal that LBPAE can navigate the multiple extra- and intra-cellular barriers involved in the fibroblast gene transfection. More importantly, LBPAE can effectively deliver a variety of genes (e.g. COL7A1) to substantially upregulate a desired expression (e.g. type VII collagen protein (C7) in HPDF), demonstrating its great potential in the treatment of diseases (e.g. C7-deficiency genodermatosis such as recessive dystrophic epidermolysis bullosa (RDEB)).

### Polymers

In some embodiments, the present disclosure provides polymers made by a process of:

(a) reacting a compound of formula (A)

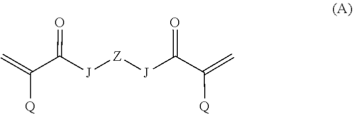

with a first amine having the formula R₁—NH₂ or R₁—N(H)—Z′—N(H)—R₁;
(b) reacting the product of Step (a) with a second amine having the formula R₂—NH₂ or R₂—N(H)—Z″—N(H)—R₂; and
(c) reacting the product of Step (b) with a compound of formula (B):

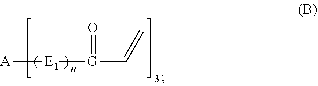

wherein

each J is independently —O— or —NH—;

Z, Z′, and Z″ are linking moieties;

A is a linear or branched carbon chain of 1 to 30 carbon atoms, a linear or branched heteroatom-containing carbon chains of 2 to 30 atoms, a carbocycle containing 3 to 30 carbon atoms, or a heterocycle containing 3 to 30 atoms;

wherein A is optionally substituted with one or more halogen, hydroxyl, amino group, sulfonyl group, sulphonamide group, thiol, C₁-C₆ alkyl, C₁-C₆ alkoxy, C₁-C₆ether, C₁-C₆ thioether, C₁-C₆ sulfone, C₁-C₆ sulfoxide, C₁-C₆ primary amide, C₁-C₆ secondary amide, halo C₁-C₆ alkyl, carboxyl group, cyano group, nitro group, nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl or C₆-C₁₀ aryl; wherein each R′ is independently selected, from the group consisting of hydrogen and C₁-C₆ alkyl;

G is —C—, —S—, —S(O)—, —P(OR₁)—, or —P(OH)—;

each Q is H or a C₁-C₁₀ linear or branched alkyl group;

each E₁ is independently selected from the group consisting of covalent bond, —N—, —O—, —S—, alkylene, heteroalkylene, alkenyl, heteroalkenylene, alkynyl, heteroalkynylene;

R₁ and R₂ are each independently C₁-C₄₀alkyl, heteroalkyl, C₂-C₄₀alkenyl, C₂-C₄₀ heteroalkenylene, C₄-C₈cycloalkenyl, C₂-C₄₀alkynyl, C₂-C₄₀ heteroalkynylene, C₃-C₈cycloalkyl, heterocyclyl, aryl, or heteroaryl; wherein the heterocyclyl and heteroaryl contain 1-5 heteroatoms selected from the group consisting of N, S, P and O; wherein the C₁-C₄₀alkyl, C₂-C₄₀alkenyl, C₄-C₈cycloalkenyl, C₂-C₄₀alkynyl, C₃-C₈cycloalkyl, heterocyclyl, aryl, and heteroaryl are optionally substituted with D, halogen, C₁-C₆alkyl, —OH, —NH(C₁-C₆alkyl), or —N(C₁-C₆alkyl)₂; and R₁ is unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ ether, a C₁-C₆ thioether, a C₁-C₆ sulfone, a C₁-C₆ sulfoxide, a C₁-C₆ primary amide, a C₁-C₆ secondary amide, a halo C₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl and C₆-C₁₀) aryl; wherein each R′ is independently selected, from the group consisting of hydrogen and C₁-C₆ alkyl; and

each n is at least 1.

In some embodiments, the present disclosure provides polymers made by a process of:

(a) reacting a compound of formula (A)

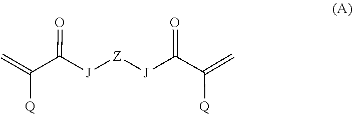

and a compound of formula (B):

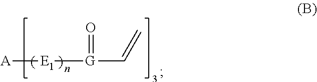

with a first amine having the formula R₁—NH₂ or R₁—N(H)—Z′—N(H)—R₁;
(b) reacting the product of Step (a) with a second amine having the formula R₂—NH₂ or R₂—N(H)—Z″—N(H)—R₂;
wherein

each J is independently —O— or —NH—;

Z, Z′, and Z″ are linking moieties;

A is a linear or branched carbon chain of 1 to 30 carbon atoms, a linear or branched heteroatom-containing carbon chains of 2 to 30 atoms, a carbocycle containing 3 to 30 carbon atoms, or a heterocycle containing 3 to 30 atoms;

wherein A is optionally substituted with one or more halogen, hydroxyl, amino group, sulfonyl group, sulphonamide group, thiol, C₁-C₆ alkyl, C₁-C₆ alkoxy, C₁-C₆ether, C₁-C₆ thioether, C₁-C₆ sulfone, C₁-C₆ sulfoxide, C₁-C₆ primary amide, C₁-C₆ secondary amide, halo C₁-C₆ alkyl, carboxyl group, cyano group, nitro group, nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl or C₆-C₁₀ aryl; wherein each R′ is independently selected, from the group consisting of hydrogen and C₁-C₆ alkyl;

G is —C—, —S—, —S(O)—, —P(OR₁)—, or —P(OH)—;

each Q is H or a C₁-C₁₀ linear or branched alkyl group;

each E₁ is independently selected from the group consisting of covalent bond, —N—, —O—, —S—, alkylene, heteroalkylene, alkenyl, heteroalkenylene, alkynyl, heteroalkynylene;

R₁ and R2 are each independently C₁-C₄₀alkyl, heteroalkyl, C₂-C₄₀alkenyl, C₂-C₄₀ heteroalkenylene, C₄-C₈cycloalkenyl, C₂-C₄₀alkynyl, C₂-C₄₀ heteroalkynylene, C₃-C₈cycloalkyl, heterocyclyl, aryl, or heteroaryl; wherein the heterocyclyl and heteroaryl contain 1-5 heteroatoms selected from the group consisting of N, S, P and O; wherein the C₁-C₄₀alkyl, C₂-C₄₀alkenyl, C₄-C₈cycloalkenyl, C₂-C₄₀alkynyl, C₃-C₈cycloalkyl, heterocyclyl, aryl, and heteroaryl are optionally substituted with D, halogen, C₁-C₆alkyl, —OH, —NH(C₁-C₆alkyl), or —N(C₁-C₆alkyl)₂; and R₁ is unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ ether, a C₁-C₆ thioether, a C₁-C₆ sulfone, a C₁-C₆ sulfoxide, a C₁-C₆ primary amide, a C₁-C₆ secondary amide, a halo C₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl and C₆-C₁₀) aryl; wherein each R′ is independently selected, from the group consisting of hydrogen and C₁-C₆ alkyl; and

each n is at least 1.

In some embodiments, Z is a linear or branched carbon chain of 1 to 30 carbon atoms, a linear or branched heteroatom-containing carbon chains of 1 to 30 atoms, a carbocycle containing 3 to 30 carbon atoms, an alkylene-carbocycle containing 3 to 30 carbon atoms, a heterocycle containing 3 to 30 atoms, or an alkylene-heterocycle containing 3 to 30 atoms. Z may be unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ether, a C₁-C₆ thioether, a C₁-C₆ sulfone, a C₁-C₆ sulfoxide, a C₁-C₆primary amide, a C₁-C₆ secondary amide, a halo C₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl and C₆-C₁₀ aryl; wherein each R′ is independently selected, from the group consisting of hydrogen and C₁-C₆ alkyl. In some embodiments, Z is a linear carbon chain of 1 to 30 carbon atoms. For example, Z may be an alkylene group including, but not limited to, C₁-C₂₄ alkylene, C₁-C₂₀ alkylene, C₁-C₁₆ alkylene, C₁-C₁₂ alkylene, C₁-C₈ alkylene, C₁-C₆ alkylene, C₁-C₄ alkylene, C₁-C₃ alkylene, C₁-C₂ alkylene, C₁ alkylene. Representative alkylene groups include, but are not limited to, methylene, ethylene, propylene, n-butylene, ethenylene, propenylene, n-butenylene, propynylene, n-butynylene, and the like. In some embodiments, Z is a linear or branched carbon chain of 1 to 30 carbon atoms or a linear or branched heteroatom-containing carbon chains of 1 to 30 atoms. In some embodiments, Z is a linear or branched carbon chain of 1 to 10 carbon atoms. For example, in some embodiments, Z is

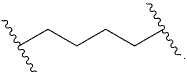 [SMILES: *CCCC*]

In some embodiments, Z is a branched carbon chain of 1 to 30 carbon atoms. In some embodiments, Z is a linear or branched heteroatom-containing carbon chain of 1 to 30 atoms. For example, Z may be a linear or branched carbon chain with one or more of the carbon atoms substituted with a heteroatom, including but not limited to O, N, S, or P. In some embodiments, is Z a carbocycle containing 3 to 30 carbon atoms. In some embodiments, Z is an alkylene-carbocycle containing 3 to 30 carbon atoms. For example, in some embodiments, Z is

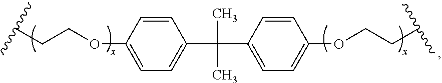

wherein x is 1-1000. In some embodiments, Z is a heterocycle containing 3 to 30 atoms. In some embodiments, Z is an alkylene-heterocycle containing 3 to 30 atoms. In some embodiments, Z is unsubstituted. In some embodiments, Z is substituted. In some embodiments, Z is one of the following

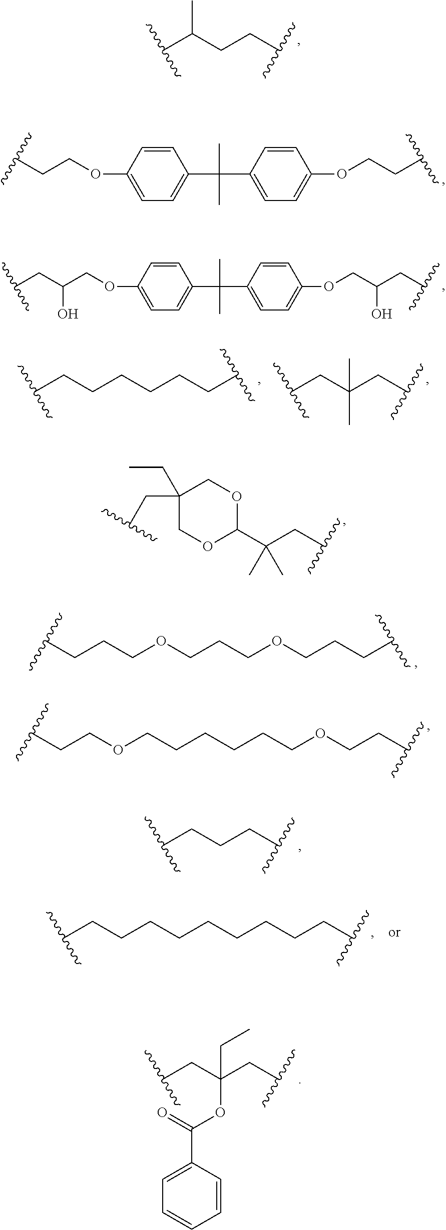

In some embodiments, Z′ is a linear or branched carbon chain of 1 to 30 carbon atoms, a linear or branched heteroatom-containing carbon chains of 1 to 30 atoms, a carbocycle containing 3 to 30 carbon atoms, an alkylene-carbocycle containing 3 to 30 carbon atoms, a heterocycle containing 3 to 30 atoms, or an alkylene-heterocycle containing 3 to 30 atoms. Z′ may be unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ether, a C₁-C₆ thioether, a C₁-C₆ sulfone, a C₁-C₆ sulfoxide, a C₁-C₆primary amide, a C₁-C₆ secondary amide, a halo C₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl and C₆-C₁₀ aryl; wherein each R′ is independently selected, from the group consisting of hydrogen and C₁-C₆ alkyl. In some embodiments, Z′ is a linear carbon chain of 1 to 30 carbon atoms. For example, Z′ may be an alkylene group including, but not limited to, C₁-C₂₄ alkylene, C₁-C₂₀ alkylene, C₁-C₁₆ alkylene, C₁-C₁₂ alkylene, C₁-C₈ alkylene, C₁-C₆ alkylene, C₁-C₄ alkylene, C₁-C₃ alkylene, C₁-C₂ alkylene, C₁ alkylene. Representative alkylene groups include, but are not limited to, methylene, ethylene, propylene, n-butylene, ethenylene, propenylene, n-butenylene, propynylene, n-butynylene, and the like. In some embodiments, Z′ is a linear or branched carbon chain of 1 to 30 carbon atoms or a linear or branched heteroatom-containing carbon chains of 1 to 30 atoms.

In some embodiments, Z″ is a linear or branched carbon chain of 1 to 30 carbon atoms, a linear or branched heteroatom-containing carbon chains of 1 to 30 atoms, a carbocycle containing 3 to 30 carbon atoms, an alkylene-carbocycle containing 3 to 30 carbon atoms, a heterocycle containing 3 to 30 atoms, or an alkylene-heterocycle containing 3 to 30 atoms. Z″ may be unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ether, a C₁-C₆ thioether, a C₁-C₆ sulfone, a C₁-C₆ sulfoxide, a C₁-C₆primary amide, a C₁-C₆ secondary amide, a halo C₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl and C₆-C₁₀ aryl; wherein each R′ is independently selected, from the group consisting of hydrogen and C₁-C₆ alkyl. In some embodiments, Z″ is a linear carbon chain of 1 to 30 carbon atoms. For example, Z″ may be an alkylene group including, but not limited to, C₁-C₂₄ alkylene, C₁-C₂₀ alkylene, C₁-C₁₆ alkylene, C₁-C₁₂ alkylene, C₁-C₈ alkylene, C₁-C₆ alkylene, C₁-C₄ alkylene, C₁-C₃ alkylene, C₁-C₂ alkylene, C₁ alkylene. Representative alkylene groups include, but are not limited to, methylene, ethylene, propylene, n-butylene, ethenylene, propenylene, n-butenylene, propynylene, n-butynylene, and the like. In some embodiments, Z″ is a linear or branched carbon chain of 1 to 30 carbon atoms or a linear or branched heteroatom-containing carbon chains of 1 to 30 atoms.

In accordance with certain embodiments of the present disclosure, G is —C—, —S—, —S(O)—, —P(OR₁)—, or —P(OH)—, thus forming a carbonyl, sulfoxide, sulfone, and phosphono group, respectively. Thus, in some embodiments, G is —C—. In some embodiments, G is —S—. In some embodiments, G is —S(O)—.

In some embodiments, the compound of formula (B) is

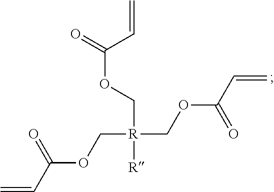

wherein

R is a linear or branched carbon chain of 1 to 10 carbon atoms, a linear or branched heteroatom-containing carbon chains of 1 to 10 atoms, a carbocycle containing 3 to 10 carbon atoms, or a heterocycle containing 3 to 10 atoms, and R is unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ ether, a C₁-C₆ thioether, a C₁-C₆ sulfone, a C₁-C₆ sulfoxide, a C₁-C₆ primary amide, a C₁-C₆ secondary amide, a halo C₁-C₆alkyl, a carboxyl group, a cyano group, a nitro group, a nitroso group, —OC(O)NR′R′, —N(R′)C(O)NR′R′, N(R′)C(O)O—C₁-C₆alkyl, C₃-C₆ cycloalkyl, C₃-C₆ heterocyclyl, C₂-C₅ heteroaryl and C₆-C₁₀ aryl; wherein each R′ is independently selected, from the group consisting of hydrogen and C₁-C₆ alkyl; and R″ is an unsubstituted or substituted, linear or branched carbon chain of 1 to 10 carbon atoms, a linear or branched heteroatom-containing carbon chains of 1 to 10 atoms, a carbocycle containing 3 to 10 carbon atoms, or a heterocycle containing 3 to 10 atoms. In some embodiments, R is 1 carbon atom. In some embodiments, R″ is a linear or branched carbon chain, such as methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, n-pentyl, isopentyl, neopentyl, n-hexyl. For example, in some embodiments, the compound of formula (B) is

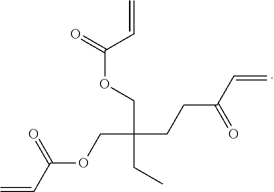 [SMILES: C=CC(=O)CCC(CC)(COC(=O)C=C)COC(=O)C=C]

In some embodiments, R is a carbocycle containing 3 to 10 carbon atoms. For example R may be cyclopropyl, cyclobutyl, cyclopentyl, cyclohexyl, cycloheptyl, cyclooctyl, cyclononyl, cyclodecyl, phenyl, or naphthyl. In some embodiments, R is a heterocycle containing 3 to 10 atoms.

In certain embodiments, the first amine has the formula R₁—NH₂ or R₁—N(H)—Z′—N(H)—R₁. In some embodiments, the first amine has the formula R₁—NH₂. In some embodiments, the first amine has the formula R₁—N(H)—Z′—N(H)—R₁. In some embodiments, the first amine having the formula R₁—N(H)—Z′—N(H)—R₁ is

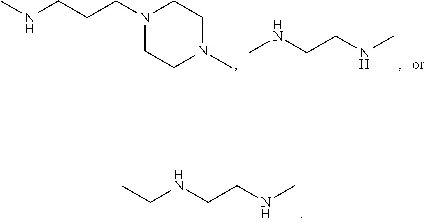

In some embodiments, the first amine has the formula R₁—N(H)—Z′—N—(R₁)₂. In some embodiments, the first amine having the formula R₁—N(H)—Z′—N—(R₁)₂ is

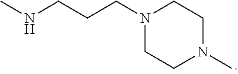

In certain embodiments, the second amine has the formula R₂—NH₂ or R₂—N(H)—Z″—N(H)—R₂. In some embodiments, the second amine has the formula R₂—NH₂. In some embodiments, the second amine has the formula R₂—N(H)—Z″—N(H)—R₂. In some embodiments, the second amine having the formula R₂—N(H)—Z″—N(H)—R₂ is

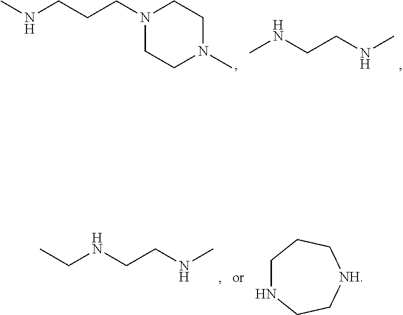

In some embodiments, the first amine has the formula R₂—N(H)—Z″—N—(R₂)₂. In some embodiments, the first amine having the formula R₂—N(H)—Z″—N—(R₂)₂ is

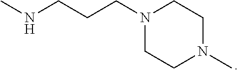

In certain embodiments, R₁ is C₁-C₄₀alkyl, C₁-C₄₀ heteroalkyl, C₂-C₄₀alkenyl, C₂-C₄₀ heteroalkenylene, C₄-C₈cycloalkenyl, C₂-C₄₀alkynyl, C₂-C₄₀ heteroalkynylene, C₃-C₈cycloalkyl, heterocyclyl, aryl, or heteroaryl; wherein the heterocyclyl and heteroaryl contain 1-5 heteroatoms selected from the group consisting of N, S, P and O; wherein the C₁-C₄₀alkyl, C₂-C₄₀alkenyl, C₄-C₈cycloalkenyl, C₂-C₄₀alkynyl, C₃-C₈cycloalkyl, heterocyclyl, aryl, and heteroaryl are optionally substituted with D, halogen, C₁-C₆alkyl, —OH, —O—C₁-C₆alkyl, —NH₂, —NH(C₁-C₆alkyl), or —N(C₁-C₆alkyl)₂. R₁ may be unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ ether, a C₁-C₆ thioether, a C₁-C₆ sulfone, a C₁-C₆ sulfoxide, a C₁-C₆ primary amide, a C₁-C₆ secondary amide, a halo C₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl and C₆-C₁₀) aryl; wherein each R′ is independently selected, from the group consisting of hydrogen and C₁-C₆ alkyl. In some embodiments, R₁ is C₁-C₂₀ alkyl. For example, R₁ may be C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl groups such as such as methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, n-pentyl, isopentyl, neopentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, or n-icosyl. In some embodiments, R₁ is unsubstituted. In some embodiments, R₁ is substituted. In some embodiments, R₁ is selected from the group consisting of

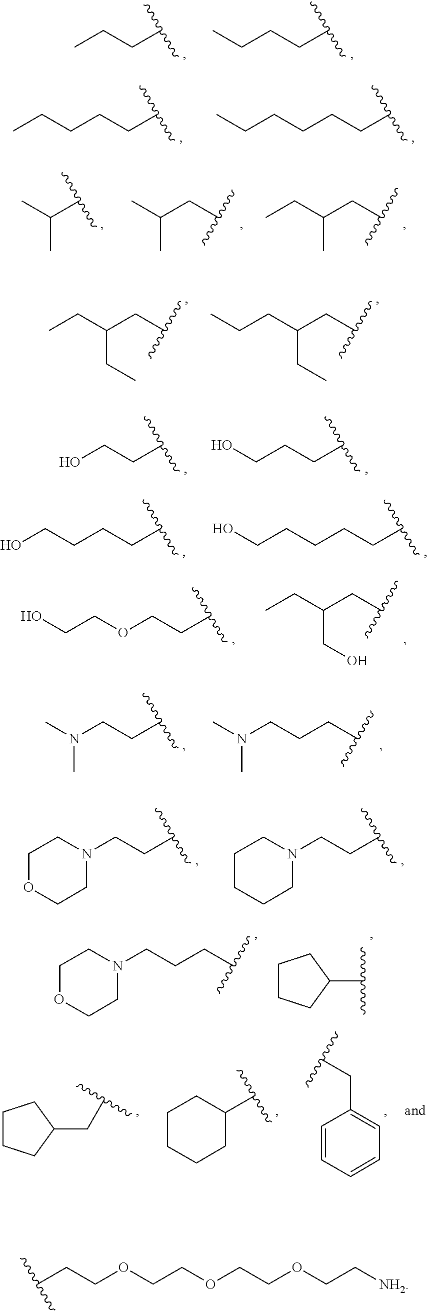

In some embodiments, R₁ is

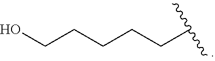 [SMILES: *CCCCCO]

In some embodiments, R₁ is

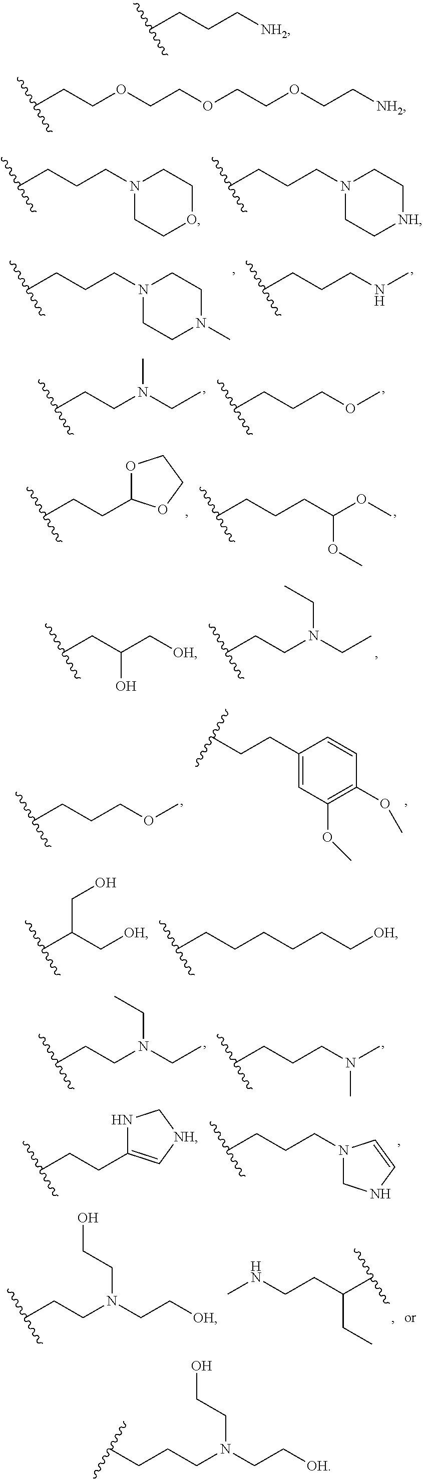

In certain embodiments, R₂ is C₁-C₄₀alkyl, C₁-C₄₀ heteroalkyl, C₂-C₄₀alkenyl, C₂-C₄₀ heteroalkenylene, C₄-C₈cycloalkenyl, C₂-C₄₀alkynyl, C₂-C₄₀ heteroalkynylene, C₃-C₈cycloalkyl, heterocyclyl, aryl, or heteroaryl; wherein the heterocyclyl and heteroaryl contain 1-5 heteroatoms selected from the group consisting of N, S, P and O; wherein the C₁-C₄₀alkyl, C₂-C₄₀alkenyl, C₄-C₈cycloalkenyl, C₂-C₄₀alkynyl, C₃-C₈cycloalkyl, heterocyclyl, aryl, and heteroaryl are optionally substituted with D, halogen, C₁-C₆alkyl, —OH, —O—C₁-C₆alkyl, —NH₂, —NH(C₁-C₆alkyl), or —N(C₁-C₆alkyl)₂. R₂ may be unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ ether, a C₁-C₆ thioether, a C₁-C₆ sulfone, a C₁-C₆ sulfoxide, a C₁-C₆ primary amide, a C₁-C₆ secondary amide, a halo C₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl and C₆-C₁₀) aryl; wherein each R′ is independently selected, from the group consisting of hydrogen and C₁-C₆ alkyl. In some embodiments, R₂ is C₁-C₂₀ alkyl. For example, R₂ may be C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl groups such as such as methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, n-pentyl, isopentyl, neopentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, or n-icosyl. In some embodiments, R₂ is unsubstituted. In some embodiments, R₂ is substituted. In some embodiments, R₂ is selected from the group consisting of

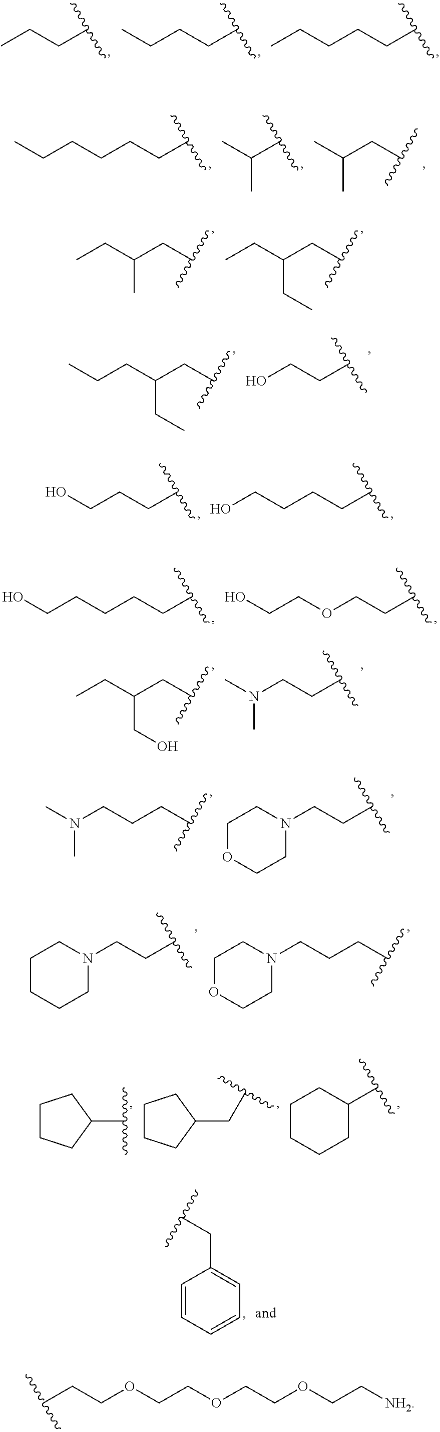

In some embodiments, R₂ is

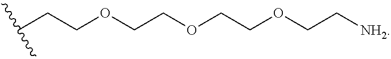 [SMILES: *CCOCCOCCOCCN]

In some embodiments, R₂ is

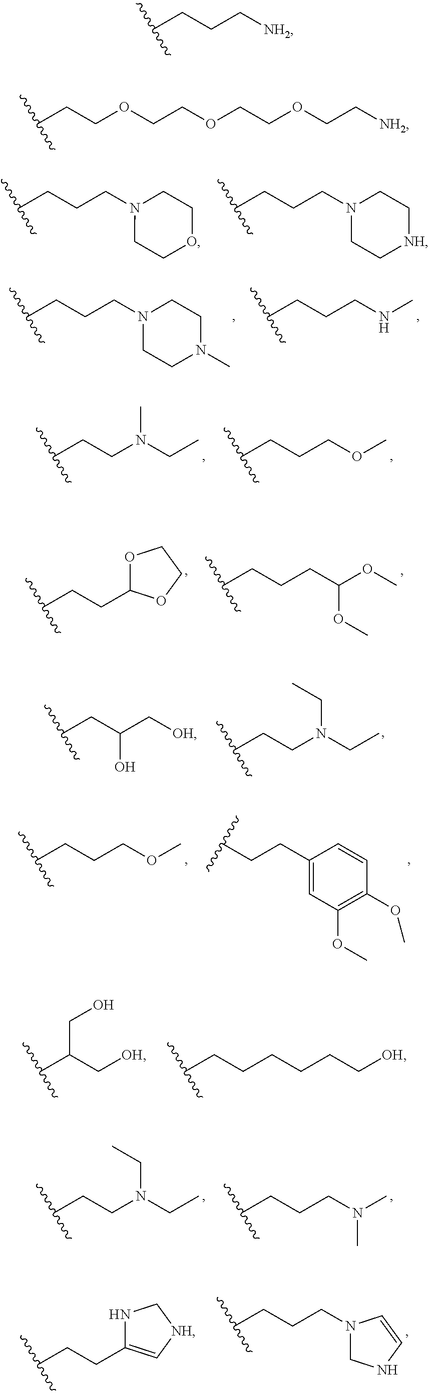

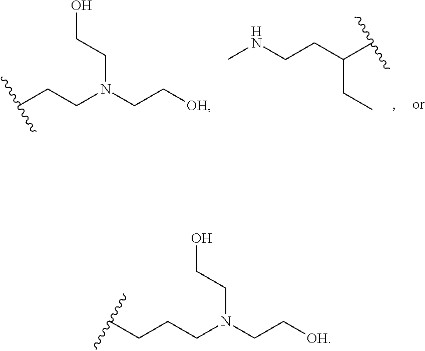

In some embodiments, each Q is H or a C₁-C₁₀ linear or branched alkyl group. Thus, in some embodiments, each Q is H. In other embodiments, each Q is a C₁-C₁₀ linear or branched alkyl group. For example, each Q may be methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, n-pentyl, isopentyl, neopentyl, n-hexyl, n-heptyl, n-octyl, n-nonyl, or n-decyl. In some embodiments, each Q is methyl.

In some embodiments, each J is —O—. In some embodiments, each J is —NH—.

In some embodiments, each E₁ is independently selected from the group consisting of covalent bond, —N—, —O—, —S—, alkylene, heteroalkylene, alkenyl, heteroalkenylene, alkynyl, heteroalkynylene. In some embodiments, each E₁ is heteroalkylene. In some embodiments, each E₁ is —CH₂—O—. In some embodiments, each n is at least 1. For example, n may be 1, 2, 3, 4, 5, 6, 7, 8, 9, or 10. In some embodiments, n is 1.

In some embodiments, A is a linear or branched carbon chain of 1 to 30 carbon atoms, a linear or branched heteroatom-containing carbon chains of 2 to 30 atoms, a carbocycle containing 3 to 30 carbon atoms, or a heterocycle containing 3 to 30 atoms. For example, in some embodiments, A is

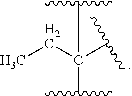 [SMILES: *C(*)(*)CC]

In some embodiments, the polymer of the present disclosure has the general structure of

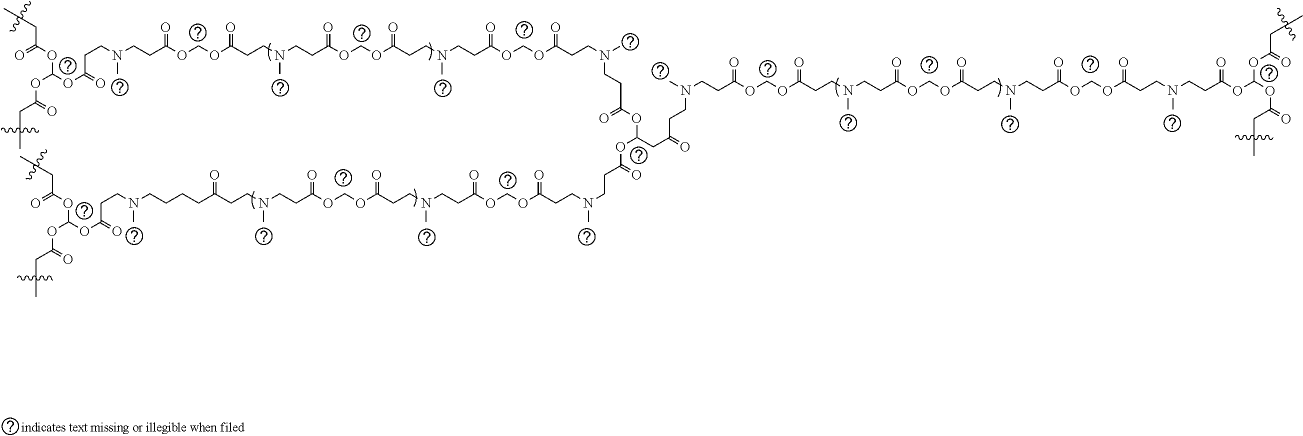

wherein the wavy bond ( [symbol] ) represents a bond to rest of the polymer, and wherein R₁, R₂, and R₄ have any of the definitions provided herein. Because of the highly controlled sequential linear oligomer growth and branching, the resulting polymers have a more uniform distribution of the linear segments and branching units, as illustrated in the above structure. As described in subsequent sections and examples, the polymers possess a strong DNA binding affinity and can condense DNA to formulate nanosized polyplexes with nearly 100% cellular uptake efficiency. In some embodiments, the polymer of the present disclosure is
wherein the groups R₂ have any of the definitions provided herein.

In some embodiments, the present disclosure provides a polymer comprising:

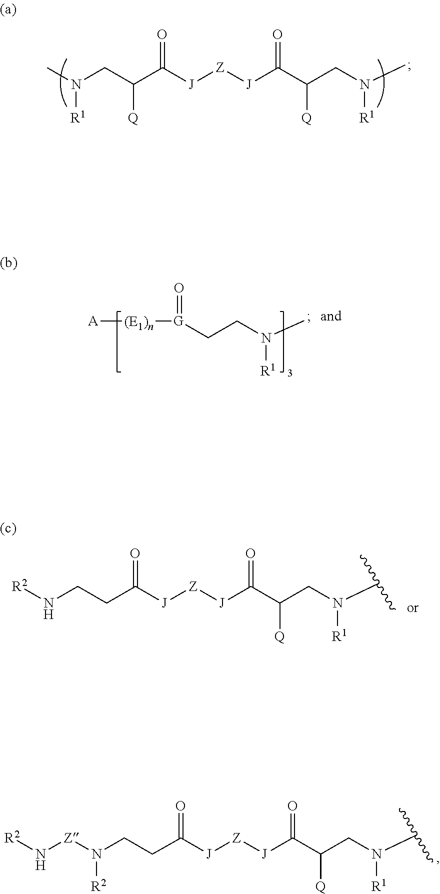

wherein R₁, R₂, A, E₁, G, J, Q, Z, Z″ and n have any of the definitions provided herein. In some further embodiments, the polymer has a M_{W} of about 3 kDa to about 200 kDa. In some further embodiments, the polymer has a M_{W} of about 5 kDa to about 50 kDa. In some further embodiments, the polymer has a M_{W} of between about 10 kDa and 50 kDa. In some further embodiments, the polymer has a M_{W} of about 5 kDa to about 15 kDa. In some further embodiments, the polymer has a M_{W} of about 10 kDa. In some further embodiments, the polymer has a M_{W} of about 20 kDa. In some further embodiments, the polymer has a M_{W} of about 30 kDa. In some further embodiments, the polymer has a M_{W} of about 40 kDa. In some further embodiments, the polymer has an alpha parameter defined from the Mark-Houwink of less than about 0.5. In some further embodiments, the polymer has an alpha parameter defined from the Mark-Houwink equation ranging from about 0.3 to about 0.5. In some further embodiments, the polymer has a PDI from about 1.0 to about 8.0. In some further embodiments, the polymer has a PDI of about 2.5.

In some embodiments, the present disclosure provides a polymer comprising:

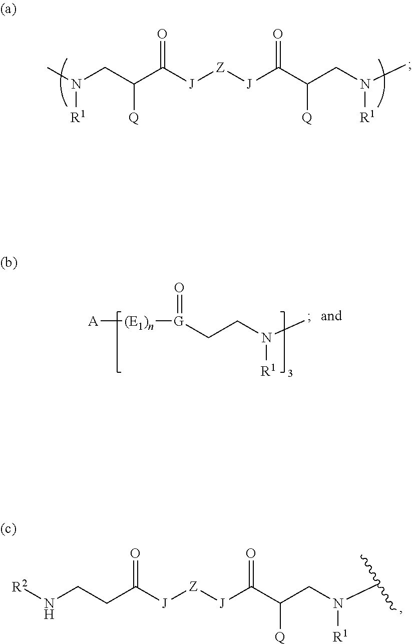

wherein R₁, R₂, A, E₁, G, J, Q, Z, and n have any of the definitions provided herein. In some further embodiments, the polymer has a M_{W} of about 3 kDa to about 200 kDa. In some further embodiments, the polymer has a M_{W} of about 5 kDa to about 50 kDa. In some further embodiments, the polymer has a M_{W} of between about 10 kDa and 50 kDa. In some further embodiments, the polymer has a M_{W} of about 5 kDa to about 15 kDa. In some further embodiments, the polymer has a M_{W} of about 10 kDa. In some further embodiments, the polymer has a M_{W} of about 20 kDa. In some further embodiments, the polymer has a M_{W} of about 30 kDa. In some further embodiments, the polymer has a M_{W} of about 40 kDa. In some further embodiments, the polymer has an alpha parameter defined from the Mark-Houwink of less than about 0.5. In some further embodiments, the polymer has an alpha parameter defined from the Mark-Houwink equation ranging from about 0.3 to about 0.5. In some further embodiments, the polymer has a PDI from about 1.0 to about 8.0. In some further embodiments, the polymer has a PDI of about 2.5.

In some embodiments, the present disclosure provides a polymer comprising:

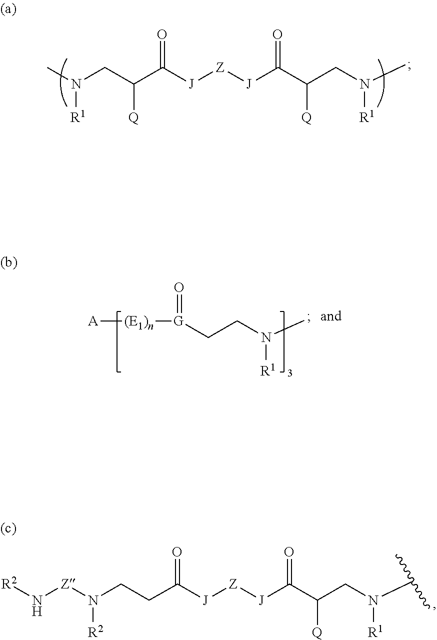

wherein R₁, R₂, A, E₁, G, J, Q, Z, Z″ and n have any of the definitions provided herein. In some further embodiments, the polymer has a M_{W} of about 3 kDa to about 200 kDa. In some further embodiments, the polymer has a M_{W} of about 5 kDa to about 50 kDa. In some further embodiments, the polymer has a M_{W} of between about 10 kDa and 50 kDa. In some further embodiments, the polymer has a M_{W} of about 5 kDa to about 15 kDa. In some further embodiments, the polymer has a M_{W} of about 10 kDa. In some further embodiments, the polymer has a M_{W} of about 20 kDa. In some further embodiments, the polymer has a M_{W} of about 30 kDa. In some further embodiments, the polymer has a M_{W} of about 40 kDa. In some further embodiments, the polymer has an alpha parameter defined from the Mark-Houwink of less than about 0.5. In some further embodiments, the polymer has an alpha parameter defined from the Mark-Houwink equation ranging from about 0.3 to about 0.5. In some further embodiments, the polymer has a PDI from about 1.0 to about 8.0. In some further embodiments, the polymer has a PDI of about 2.5.

In some further embodiments, the polymer comprises:

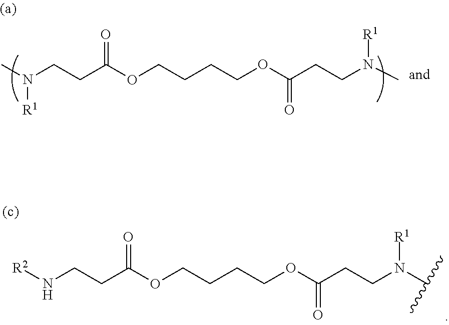

In some further embodiments, the polymer comprises:

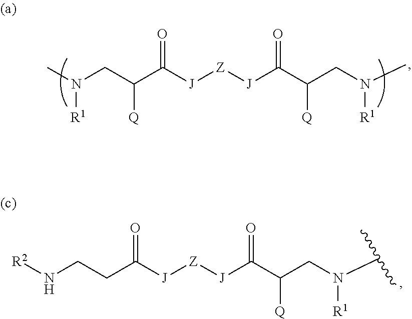

and
wherein

J is O and Z is

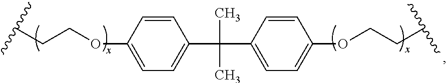

wherein x is 1-1000.

In some further embodiments, the polymer comprises:

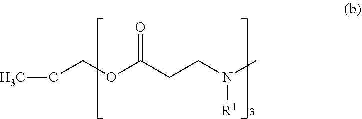

In some further embodiments, R₁ is selected from

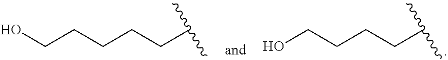 [SMILES: *CCCCCO ; *CCCCO]

In some further embodiments, R₁ is

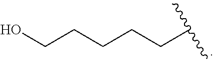 [SMILES: *CCCCCO]

In some further embodiments, R₁ is

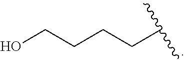 [SMILES: *CCCCO]

In some further embodiments, R₂ is selected from

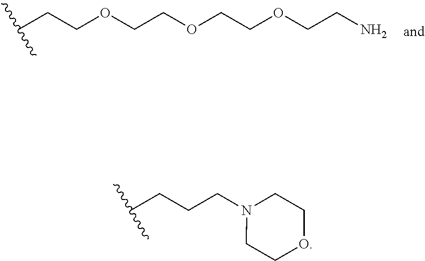 [SMILES: *CCOCCOCCOCCN ; *CCCN1CCOCC1]

In some further embodiments, R₂ is

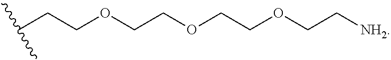 [SMILES: *CCOCCOCCOCCN]

In some further embodiments, R₂ is

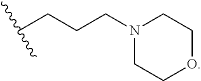 [SMILES: *CCCN1CCOCC1]

In some further embodiments, R₁ is

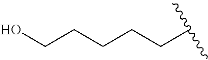 [SMILES: *CCCCCO]

### and R₂ is

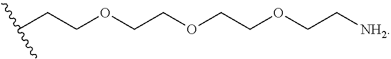 [SMILES: *CCOCCOCCOCCN]

In some further embodiments, R₁ is

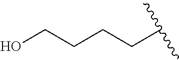 [SMILES: *CCCCO]

### and R₂ is

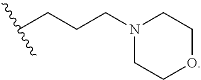 [SMILES: *CCCN1CCOCC1]

In some embodiments, the polymer comprises:

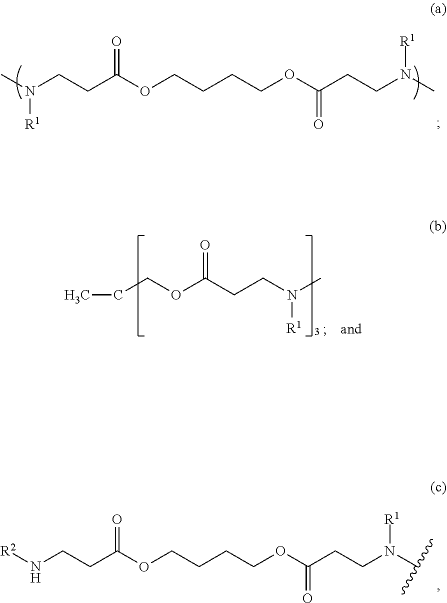

wherein

### R₁ is

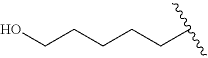 [SMILES: *CCCCCO]

and
R₂ is selected from

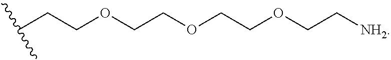 [SMILES: *CCOCCOCCOCCN]

In some further embodiments, the polymer has a M_{W} of about 3 kDa to about 200 kDa. In some further embodiments, the polymer has a M_{W} of about 5 kDa to about 50 kDa. In some further embodiments, the polymer has a M_{W} of between about 10 kDa and 50 kDa. In some further embodiments, the polymer has a M_{W} of about 5 kDa to about 15 kDa. In some further embodiments, the polymer has a M_{W} of about 10 kDa. In some further embodiments, the polymer has a M_{W} of about 20 kDa. In some further embodiments, the polymer has a M_{W} of about 30 kDa. In some further embodiments, the polymer has a M_{W} of about 40 kDa. In some further embodiments, the polymer has an alpha parameter defined from the Mark-Houwink of less than about 0.5. In some further embodiments, the polymer has an alpha parameter defined from the Mark-Houwink equation ranging from about 0.3 to about 0.5. In some further embodiments, the polymer has a PDI from about 1.0 to about 8.0. In some further embodiments, the polymer has a PDI of about 2.5.

In some embodiments, the polymer comprises:

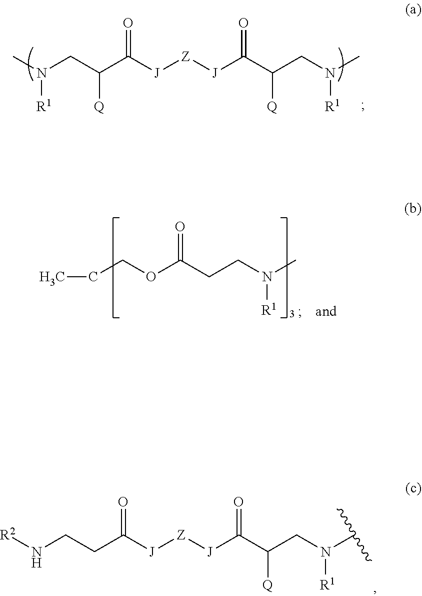

wherein

J is O and Z is

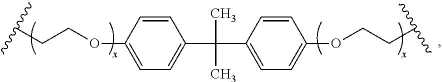

wherein x is 1-1000;

R₁ is

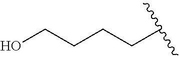 [SMILES: *CCCCO]

and

R₂ is

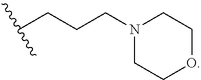 [SMILES: *CCCN1CCOCC1]

In some further embodiments, the polymer has a M_{W} of about 3 kDa to about 200 kDa. In some further embodiments, the polymer has a M_{W} of about 5 kDa to about 50 kDa. In some further embodiments, the polymer has a M_{W} of between about 10 kDa and 50 kDa. In some further embodiments, the polymer has a M_{W} of about 5 kDa to about 15 kDa. In some further embodiments, the polymer has a M_{W} of about 10 kDa. In some further embodiments, the polymer has a M_{W} of about 20 kDa. In some further embodiments, the polymer has a M_{W} of about 30 kDa. In some further embodiments, the polymer has a M_{W} of about 40 kDa. In some further embodiments, the polymer has an alpha parameter defined from the Mark-Houwink of less than about 0.5. In some further embodiments, the polymer has an alpha parameter defined from the Mark-Houwink equation ranging from about 0.3 to about 0.5. In some further embodiments, the polymer has a PDI from about 1.0 to about 8.0. In some further embodiments, the polymer has a PDI of about 2.5.

In some embodiments, certain polymers of the present disclosure can be described as linear polymers (oligomers) of compounds of formula (A) and a first amine having the formula R₁—NH₂ or R₁—N(H)—Z′—N(H)—R₁ (as described herein), crosslinked with compounds of formula (B) (as described herein). When the linear oligomers are prepared under conditions in which the compounds of formula (A) are present in molar excess compared to the first amine having the formula R₁—NH₂ or R₁—N(H)—Z′—N(H)—R₁, the resulting oligomeric species are terminated with Michael acceptor groups (e.g., and acrylate, methacrylate, acrylamide, or other such group), and can be subsequently end-capped under appropriate conditions with a second amine having the formula R₂—NH₂ or R₂—N(H)—Z″—N(H)—R₂ (as described herein). The resulting end-capped oligomer(s) can then be reacted with a tri-functional Michael acceptor compound of formula (B) (as described herein) to provide a branched structure. Such crosslinked polymers can be alternatively defined by the molecular weight distribution of the oligomeric segments (e.g., M_{w} values ranging from about 3 to about 200 as disclosed herein) and the molar or weight percentage of crosslinks derived from the incorporation of Michael acceptor compounds of formula (B).

In some embodiments, a molar excess of the compound of formula (A) is reacted with the first amine. For example, the stoichiometric ratio of the compound of formula (A) to the first amine may range from about 1.1:1 to about 10:1 including about 1.1:1, about 1.2:1, about 1.3:1, about 1.4:1, about 1.5:1, about 1.6:1, about 1.7:1, about 1.8:1, about 1.9:1, about 2:1, about 3:1, about 4:1, about 5:1, about 6:1, about 7:1, about 8:1, about 9:1 or about 10:1, including all ranges there between.

In some embodiments, the stoichiometric ratio of the compound of formula (A) to the first amine is about 1.1:1, about 1.2:1, about 1.3:1, about 1.4:1, about 1.5:1, about 1.6:1, about 1.7:1, about 1.8:1, about 1.9:1, about 2:1, about 3:1, about 4:1, about 5:1, about 6:1, about 7:1, about 8:1, about 9:1 or about 10:1. In some embodiments, the stoichiometric ratio of the compound of formula (A) to the first amine may range from about 1.1:1 to about 2:1. In some embodiments, the stoichiometric ratio the of the compound of formula (A) to the first amine is about 1.2:1. In some embodiments, the compound of formula (A) is reacted with the first amine at a molar equivalence (i.e. about 1:1).

In some embodiments, Step (a) is performed in an organic solvent. A wide variety of organic solvents can be used in the context of the present disclosure, including but not limited to dimethylsulfoxide (DMSO), N,N-dimethylformamide (DMF), N-methylpyrrolidone (NMP) and the like; ketones such as acetone, methyl ethyl ketone, methyl isobutyl ketone and the like; ethers such tetrahydrofuran (THF), diethylether, methyl tertiary-butyl ether and the like; hydrocarbons such as toluene, xylene, cyclohexane and the like. In some embodiments, Step (a) is performed in DMSO.

In some embodiments, Step (a) is performed at a temperature ranging from about 40° C. to about 120° C., including about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, or 120° C., including all ranges there between.

In some embodiments, Step (a) is performed at 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, or 120° C. In some embodiments, Step (a) is performed at about 90° C.

In some embodiments, the product of Step (a) is not purified before Step (b). In other embodiments, the product of Step (a) is purified before Step (b). The product of step (a) may be purified by a variety of methods and techniques apparent to a person having ordinary skill in the art.

In some embodiments, a molar excess of the second amine is added to the product of Step (a). For example, the stoichiometric ratio of the second amine to the product of Step (a) may range from about 1.1:1 to about 10:1 including about 1.1:1, about 1.2:1, about 1.3:1, about 1.4:1, about 1.5:1, about 1.6:1, about 1.7:1, about 1.8:1, about 1.9:1, about 2:1, about 3:1, about 4:1, about 5:1, about 6:1, about 7:1, about 8:1, about 9:1 or about 10:1, including all ranges there between.

In some embodiments, the stoichiometric ratio of the second amine to the product of Step (a) is about 1.1:1, about 1.2:1, about 1.3:1, about 1.4:1, about 1.5:1, about 1.6:1, about 1.7:1, about 1.8:1, about 1.9:1, about 2:1, about 3:1, about 4:1, about 5:1, about 6:1, about 7:1, about 8:1, about 9:1 or about 10:1. In some embodiments, the stoichiometric ratio of the of the second amine to the product of Step (a) is about 5:1. In some embodiments, the second amine is reacted with the product of Step (a) at a molar equivalence (i.e. about 1:1).

In some embodiments, Step (b) is performed at a temperature ranging from about 16° C. to about 40° C. For example, Step (b) is performed at a temperature ranging from about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, to about 40° C., including all ranges there between.

In some embodiments, Step (b) is performed at a temperature of about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about or about 40° C.

In some embodiments, the product of Step (b) is not purified before Step (c). In other embodiments, the product of Step (b) is purified before Step (c). The product of step (b) may be purified by a variety of methods and techniques apparent to a person having ordinary skill in the art. For example, the product of Step (b) may be purified by dialysis.

In some embodiments, Step (c) is performed at a temperature higher than that of Step (b). For example, Step (c) is performed at a temperature ranging from about 21° C. to about 200° C. For examples, Step (c) is performed at a temperature ranging from about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101 about, 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, to about 200° C., including all ranges there between.

In some embodiments, Step (c) is performed at about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101 about, 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, or about 200° C. In some embodiments, Step (c) is performed at about 90° C.

In some embodiments, the present disclosure provides a polymer of formula (I):

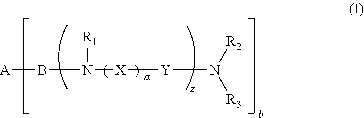

wherein

each A is independently a linear or branched carbon chain of 1 to 30 carbon atoms, a linear or branched heteroatom-containing carbon chains of 1 to 30 atoms, a carbocycle containing 3 to 30 carbon atoms, or a heterocycle containing 3 to 30 atoms;

wherein A is optionally substituted with one or more halogen, hydroxyl, amino group, sulfonyl group, sulphonamide group, thiol, C₁-C₆ alkyl, C₁-C₆ alkoxy, C₁-C₆ether, C₁-C₆ thioether, C₁-C₆ sulfone, C₁-C₆ sulfoxide, C₁-C₆primary amide, C₁-C₆ secondary amide, halo C₁-C₆ alkyl, carboxyl group, cyano group, nitro group, nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl or C₆-C₁₀ aryl; wherein each R′ is independently selected, from the group consisting of hydrogen and C₁-C₆ alkyl;

each B is independently

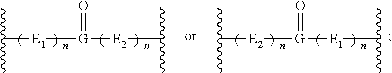

G is —C—, —S—, —S(O)—, —P(OR₁)—, or —P(OH)—; n is at least 1;

each E₁ is selected from the group consisting of covalent bond, —N—, —O—, —S—, alkylene, heteroalkylene, alkenyl, heteroalkenylene, alkynyl, heteroalkynylene;

each E₂ is selected from the group consisting of covalent bond, —N—, —O—, —S—, alkylene, heteroalkylene, alkenyl, heteroalkenylene, alkynyl, heteroalkynylene

each X is independently

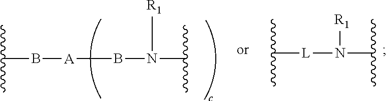

each Y is independently

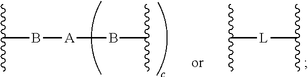

each L is independently a second linking moiety;

each R₁, R₂ and R₃ are independently, at each occurrence H, C₁-C₄₀alkyl, C₁-C₄₀ heteroalkyl, C₂-C₄₀alkenyl, C₂-C₄₀ heteroalkenylene, C₄-C₈cycloalkenyl, C₂-C₄₀alkynyl, C₂-C₄₀ heteroalkynylene, C₃-C₈cycloalkyl, heterocyclyl, aryl, or heteroaryl; wherein the heterocyclyl and heteroaryl contain 1-5 heteroatoms selected from the group consisting of N, S, P and O; wherein the C₁-C₆alkyl, C₂-C₈alkenyl, C₄-C₈cycloalkenyl, C₂-C₆alkynyl, C₃-C₈cycloalkyl, heterocyclyl, aryl, and heteroaryl are optionally substituted with D, halogen, C₁-C₆alkyl, —OH, —O—C₁-C₆alkyl, —NH₂, —NH(C₁-C₆alkyl), or —N(C₁-C₆alkyl)₂; or

wherein R₂ and R₃ together with the atom to which they are attached can form heterocyclyl or heteroaryl containing 1-3 heteroatoms selected from the group consisting of N, S, P and 0;

a is 1-1000;

b is 3 or 4;

c is 1-3; and

z is 1-100;

with the proviso that at least one of R₂ and R₃ is not H and when G is C then E₁ is not —CH₂—O—.

In certain embodiments, the present disclosure provides a polymer of formula (II):

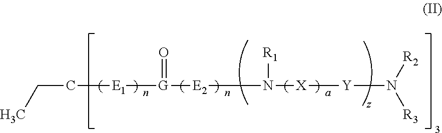

wherein,

each E₁ is selected from the group consisting of covalent bond, —N—, —O—, —S—, alkylene, heteroalkylene, alkenyl, heteroalkenylene, alkynyl, heteroalkynylene;

each E2 is selected from the group consisting of covalent bond, —N—, —O—, —S—, alkylene, heteroalkylene, alkenyl, heteroalkenylene, alkynyl, heteroalkynylene;

G is —C—, —S—, —S(O)—, —P(OR₁)—, or —P(OH)—; and

n is at least 1,

with the proviso that when G is C then E₁ is not —CH₂—O—.

In some embodiments, each E₁ and E₂ are independently selected from the group consisting of covalent bond, —N—, —S—, alkylene, heteroalkylene, alkenyl, heteroalkenylene, alkynyl, heteroalkynylene. In some embodiments, each E₁ is heteroalkylene. In some embodiments, each E₁ is —CH₂—N—. In some embodiments, each E₂ is alkylene. In some embodiments, each E₂ is

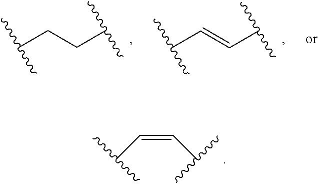

In some embodiments, each E₂ is

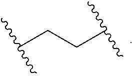 [SMILES: *CC*]

In some embodiments, each n is at least 1. For example, n may be 1, 2, 3, 4, 5, 6, 7, 8, 9, or 10. In some embodiments, n is 1. In some embodiments, G is —C—. In some embodiments, each B is

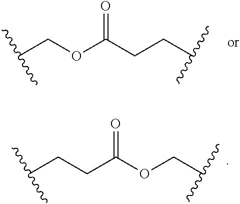 [SMILES: *CCC(=O)OC* ; *CCC(=O)OC*]

In some embodiments, each L is

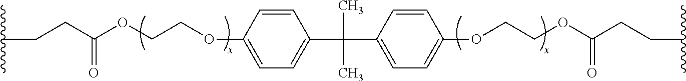

wherein x is 1-1000. In some embodiments, a is at least 2, b is 3, and each X is

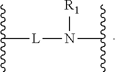

In some embodiments, each A is

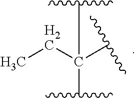 [SMILES: *C(*)(*)CC]

In some embodiments, each L is

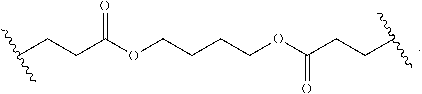 [SMILES: *CCC(=O)OCCCCOC(=O)CC*]

In some embodiments, each R₂ and/or R₃ is

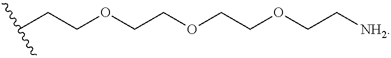 [SMILES: *CCOCCOCCOCCN]

In some embodiments, each R₁ is

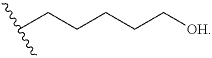 [SMILES: *CCCCCO]

In some embodiments, the present disclosure provides a polymer of formulae (III) to (VIIe):

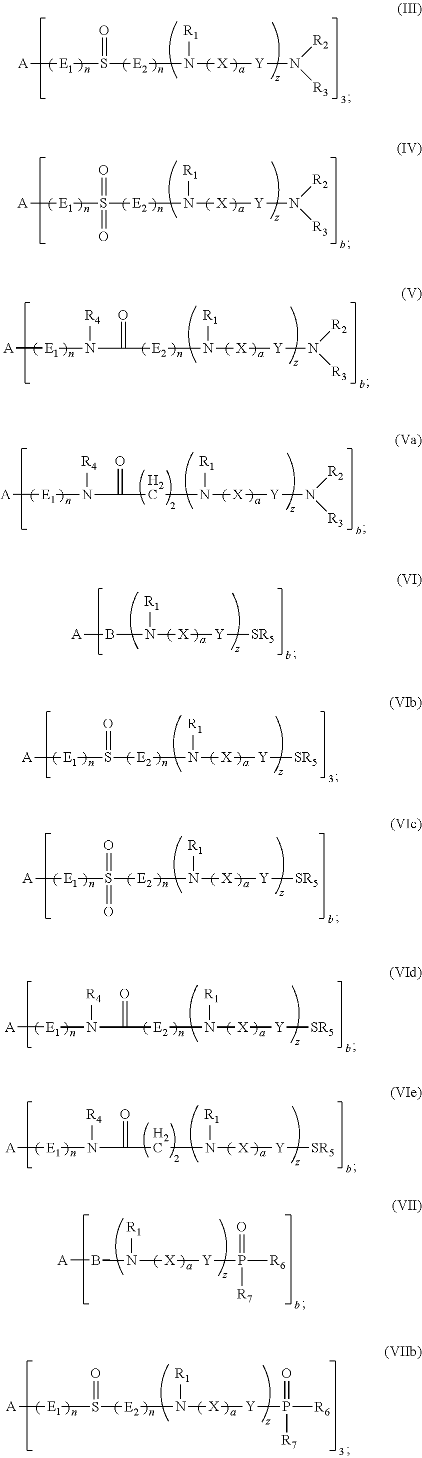

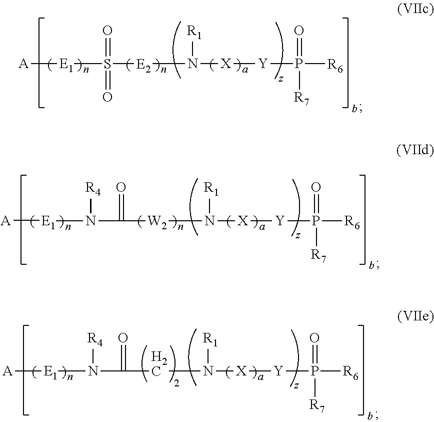

wherein each of R₅, R₆ and R₇ are independently, at each occurrence H, C₁-C₄₀alkyl, C₁-C₄₀ heteroalkyl, C₂-C₄₀alkenyl, C₂-C₄₀ heteroalkenylene, C₄-C₈cycloalkenyl, C₂-C₄₀alkynyl, C₂-C₄₀ heteroalkynylene, C₃-C₈cycloalkyl, heterocyclyl, aryl, or heteroaryl; wherein the heterocyclyl and heteroaryl contain 1-5 heteroatoms selected from the group consisting of N, S, P and O; wherein the C₁-C₆alkyl, C₂-C₈alkenyl, C₄-C₈cycloalkenyl, C₂-C₆alkynyl, C₃-C₈cycloalkyl, heterocyclyl, aryl, and heteroaryl are optionally substituted with D, halogen, C₁-C₆alkyl, —OH, —O—C₁-C₆alkyl, —NH₂, —NH(C₁-C₆alkyl), or —N(C₁-C₆alkyl)₂; and the remaining variables are as defined above.

In some embodiments, z is 1, 2, or 3. In some embodiments, z is 1.

The alpha parameter defined from the Mark-Houwink equation refers to the Mark-Houwink plot. A Mark-Houwink plot is a powerful tool for investigating polymer structure in solution as it clearly reveals the structure-molecular weight relationship with high sensitivity. It is generated by plotting the molecular weight (MW) against the intrinsic viscosity (IV) on a log-log graph. The molecular weight, of course, indicates the length of the polymer chains (or degree of polymerization) but on its own cannot give any indication of structure. The intrinsic viscosity (expressed in dL/g) is a measurement of the molecular density of the polymer chains in solution. The tighter the chains fold or coil in solution, the higher the density and the lower the intrinsic viscosity. This measurement is independent of the molecular weight, so two different structures having the same molecular weight can have different intrinsic viscosities—for example a linear (unbranched) polymer and a branched polymer of the same molecular weight will have different intrinsic viscosities. Furthermore, if the polymer changes structure across its molecular weight distribution (e.g. becomes more substituted), the intrinsic viscosity changes will be easily detected. This is what makes the Mark-Houwink plot so useful and powerful. The raw data for the Mark-Houwink plot is conveniently and simply obtained from high quality multi-detection GPC/SEC data by combining the molecular weight from a light scattering detector with the intrinsic viscosity from a viscometer detector. Both data sets are measured at each point across the elution profile of the sample. The resulting plot can be used in many ways from simply assessing how close two structures are to making complex quantitative measurements of polymer branching. In general: α<0.5: Compact/spherical chains; 0.5<α<0.8: Random-coil/flexible chains; 0.5<α<0.8: Rigid-rod/stiff chains.

In some embodiments, the polymers of the present disclosure have an alpha parameter defined from the Mark-Houwink equation of less than about 0.5. For example, the polymers of the present disclosure have an alpha parameter defined from the Mark-Houwink equation ranging from about 0.01 to about 0.49. For example, the polymers of the present disclosure have an alpha parameter defined from the Mark-Houwink equation ranging from about 0.01, about 0.02, about 0.03, about 0.04, about 0.05, about 0.06, about 0.07, about 0.08, about 0.09, about 0.10, about 0.11, about 0.12, about 0.13, about 0.14, about 0.15, about 0.16, about 0.17, about 0.18, about 0.19, about 0.20, about 0.21, about 0.22, about 0.23, about 0.24, about 0.25, about 0.26, about 0.27, about 0.28, about 0.29, about 0.30, about 0.31, about 0.32, about 0.33, about 0.34, about 0.35, about 0.36, about 0.37, about 0.38, about 0.39, about 0.40, about 0.41, about 0.42, about 0.43, about 0.44, about 0.45, about 0.46, about 0.47, about 0.48, about to about 0.49, including all ranges there between. In some embodiments, the polymers of the present disclosure have an alpha parameter defined from the Mark-Houwink equation from about 0.2 to about 0.5.

In some embodiments, the polymers of the present disclosure have an alpha parameter defined from the Mark-Houwink equation of about 0.01, about 0.02, about 0.03, about 0.04, about 0.05, about 0.06, about 0.07, about 0.08, about 0.09, about 0.10, about 0.11, about 0.12, about 0.13, about 0.14, about 0.15, about 0.16, about 0.17, about 0.18, about 0.19, about 0.20, about 0.21, about 0.22, about 0.23, about 0.24, about 0.25, about 0.26, about 0.27, about 0.28, about 0.29, about 0.30, about 0.31, about 0.32, about 0.33, about 0.34, about 0.35, about 0.36, about 0.37, about 0.38, about 0.39, about 0.40, about 0.41, about 0.42, about 0.43, about 0.44, about 0.45, about 0.46, about 0.47, about 0.48, or about 0.49.

The term “polydispersity index” (PDI) refers to a measure of the distribution of molecular mass in a given polymer sample. The polydispersity index is calculated by dividing the weight average molecular weight (Mw) by the number average molecular weight (Mn). As used herein, the term “weight average molecular weight” generally refers to a molecular weight measurement that depends on the contributions of polymer molecules according to their sizes. As used herein, the term “number average molecular weight” generally refers to a molecular weight measurement that is calculated by dividing the total weight of all the polymer molecules in a sample with the total number of polymer molecules in the sample. These terms are well-known by those of ordinary skill in the art.

In some embodiments, the polymers of the present disclosure have a PDI from about 1.01 to about 8.0. For example, the PDI may range from about 1.01, about 1.02, about 1.03, about 1.04, about 1.05, about 1.06, about 1.07, about 1.08, about 1.09, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, to about 8.0, including all ranges there between.

In some embodiments, the polymers of the present disclosure have a PDI of about 1.01, about 1.02, about 1.03, about 1.04, about 1.05, about 1.06, about 1.07, about 1.08, about 1.09, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. In some embodiments, the polymers of the present disclosure have a PDI of about 2.5.

In some embodiments, the polymers of the present disclosure have a M_{W} of at least 3 kDa. In some embodiments, the polymers of the present disclosure have a M_{W} of about 3 kDa to about 200 kDa. Accordingly, the polymers of the present disclosure have a M_{W} ranging from about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100 about, 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167 about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, to about 200 kDa, including all ranges there between. In some embodiments, the polymer has a M_{W} of between about 5 kDa and 50 kDa. In some embodiments, the polymer has a M_{W} of between about 10 kDa and 50 kDa.

In some embodiments, the polymers of the present disclosure have a M_{W} about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100 about, 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167 about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, to about 200 kDa. In some embodiments, the polymer has a M_{W} of between about 5 kDa and 50 kDa. In some embodiments, the polymer has a M_{W} of between about 10 kDa and 50 kDa. In some embodiments, the polymer has a M_{W} of about 10 kDa. In some embodiments, the polymer has a M_{W} of about 20 kDa. In some embodiments, the polymer has a M_{W} of about 30 kDa. In some embodiments, the polymer has a M_{W} of about 40 kDa.

In some embodiments, the product after Step (b) has a M_{W} of about 3 kDa. In some embodiments, the product after Step (b) has a M_{W} of about 10 kDa. In some embodiments, the product after Step (b) has a M_{W} of about 20 kDa. In some embodiments, the product after Step (b) has a M_{W} of about 30 kDa. In some embodiments, the product after Step (b) has a M_{W} of about 40 kDa.

### Methods of Making

In some embodiments, the present disclosure provides a method of making polymers comprising:

(a) reacting a compound of formula (A)

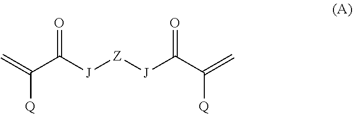

with a first amine having the formula R₁—NH₂ or R₁—N(H)—Z′—N(H)—R₁;
(b) reacting the product of Step (a) with a second amine having the formula R₂—NH₂ or R₂—N(H)—Z″—N(H)—R₂; and
(c) reacting the product of Step (b) with a compound of formula (B):

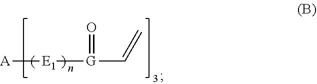

wherein

each J is independently —O— or —NH—;

Z, Z′, and Z″ are linking moieties;

A is a linear or branched carbon chain of 1 to 30 carbon atoms, a linear or branched heteroatom-containing carbon chains of 2 to 30 atoms, a carbocycle containing 3 to 30 carbon atoms, or a heterocycle containing 3 to 30 atoms;

wherein A is optionally substituted with one or more halogen, hydroxyl, amino group, sulfonyl group, sulphonamide group, thiol, C₁-C₆ alkyl, C₁-C₆ alkoxy, C₁-C₆ether, C₁-C₆ thioether, C₁-C₆ sulfone, C₁-C₆ sulfoxide, C₁-C₆ primary amide, C₁-C₆ secondary amide, halo C₁-C₆ alkyl, carboxyl group, cyano group, nitro group, nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl or C₆-C₁₀ aryl; wherein each R′ is independently selected, from the group consisting of hydrogen and C₁-C₆ alkyl;

G is —C—, —S—, —S(O)—, —P(OR₁)—, or —P(OH)—;

each Q is H or a C₁-C₁₀ linear or branched alkyl group;

each E₁ is independently selected from the group consisting of covalent bond, —N—, —O—, —S—, alkylene, heteroalkylene, alkenyl, heteroalkenylene, alkynyl, heteroalkynylene;

R₁ and R2 are each independently C₁-C₄₀alkyl, heteroalkyl, C₂-C₄₀alkenyl, C₂-C₄₀ heteroalkenylene, C₄-C₈cycloalkenyl, C₂-C₄₀alkynyl, C₂-C₄₀ heteroalkynylene, C₃-C₈cycloalkyl, heterocyclyl, aryl, or heteroaryl; wherein the heterocyclyl and heteroaryl contain 1-5 heteroatoms selected from the group consisting of N, S, P and O; wherein the C₁-C₄₀alkyl, C₂-C₄₀alkenyl, C₄-C₈cycloalkenyl, C₂-C₄₀alkynyl, C₃-C₈cycloalkyl, heterocyclyl, aryl, and heteroaryl are optionally substituted with D, halogen, C₁-C₆alkyl, —OH, —NH(C₁-C₆alkyl), or —N(C₁-C₆alkyl)₂; and R₁ is unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ ether, a C₁-C₆ thioether, a C₁-C₆ sulfone, a C₁-C₆ sulfoxide, a C₁-C₆ primary amide, a C₁-C₆ secondary amide, a halo C₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl and C₆-C₁₀) aryl; wherein each R′ is independently selected, from the group consisting of hydrogen and C₁-C₆ alkyl; and

each n is at least 1.

In some embodiments, the present disclosure provides a method of making polymers comprising:

(a) reacting a compound of formula (A)

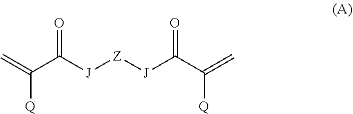

and a compound of formula (B):

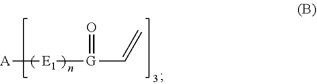

with a first amine having the formula R₁—NH₂ or R₁—N(H)—Z′—N(H)—R₁;
(b) reacting the product of Step (a) with a second amine having the formula R₂—NH₂ or R₂—N(H)—Z″—N(H)—R₂;
wherein

each J is independently —O— or —NH—;

Z, Z′, and Z″ are linking moieties;

A is a linear or branched carbon chain of 1 to 30 carbon atoms, a linear or branched heteroatom-containing carbon chains of 2 to 30 atoms, a carbocycle containing 3 to 30 carbon atoms, or a heterocycle containing 3 to 30 atoms;

wherein A is optionally substituted with one or more halogen, hydroxyl, amino group, sulfonyl group, sulphonamide group, thiol, C₁-C₆ alkyl, C₁-C₆ alkoxy, C₁-C₆ether, C₁-C₆ thioether, C₁-C₆ sulfone, C₁-C₆ sulfoxide, C₁-C₆ primary amide, C₁-C₆ secondary amide, halo C₁-C₆ alkyl, carboxyl group, cyano group, nitro group, nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl or C₆-C₁₀ aryl; wherein each R′ is independently selected, from the group consisting of hydrogen and C₁-C₆ alkyl;

G is —C—, —S—, —S(O)—, —P(OR₁)—, or —P(OH)—;

each Q is H or a C₁-C₁₀ linear or branched alkyl group;

each E₁ is independently selected from the group consisting of covalent bond, —N—, —O—, —S—, alkylene, heteroalkylene, alkenyl, heteroalkenylene, alkynyl, heteroalkynylene;

R₁ and R2 are each independently C₁-C₄₀alkyl, C₁-C₄₀ heteroalkyl, C₂-C₄₀alkenyl, C₂-C₄₀ heteroalkenylene, C₄-C₈cycloalkenyl, C₂-C₄₀alkynyl, C₂-C₄₀ heteroalkynylene, C₃-C₈cycloalkyl, heterocyclyl, aryl, or heteroaryl; wherein the heterocyclyl and heteroaryl contain 1-5 heteroatoms selected from the group consisting of N, S, P and O; wherein the C₁-C₄₀alkyl, C₂-C₄₀alkenyl, C₄-C₈cycloalkenyl, C₂-C₄₀alkynyl, C₃-C₈cycloalkyl, heterocyclyl, aryl, and heteroaryl are optionally substituted with D, halogen, C₁-C₆alkyl, —OH, —O—C₁-C₆alkyl, —NH₂, —NH(C₁-C₆alkyl), or —N(C₁-C₆alkyl)₂; and R₁ is unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ ether, a C₁-C₆ thioether, a C₁-C₆ sulfone, a C₁-C₆ sulfoxide, a C₁-C₆ primary amide, a C₁-C₆ secondary amide, a halo C₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl and C₆-C₁₀) aryl; wherein each R′ is independently selected, from the group consisting of hydrogen and C₁-C₆ alkyl; and

each n is at least 1.

In some embodiments, Z is a linear or branched carbon chain of 1 to 30 carbon atoms, a linear or branched heteroatom-containing carbon chains of 1 to 30 atoms, a carbocycle containing 3 to 30 carbon atoms, an alkylene-carbocycle containing 3 to 30 carbon atoms, a heterocycle containing 3 to 30 atoms, or an alkylene-heterocycle containing 3 to 30 atoms. Z may be unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ether, a C₁-C₆ thioether, a C₁-C₆ sulfone, a C₁-C₆ sulfoxide, a C₁-C₆primary amide, a C₁-C₆ secondary amide, a halo C₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl and C₆-C₁₀ aryl; wherein each R′ is independently selected, from the group consisting of hydrogen and C₁-C₆ alkyl. In some embodiments, Z is a linear carbon chain of 1 to 30 carbon atoms. For example, Z may be an alkylene group including but not limited to, C₁-C₂₄ alkylene, C₁-C₂₀ alkylene, C₁-C₁₆ alkylene, C₁-C₁₂ alkylene, C₁-C₈ alkylene, C₁-C₆ alkylene, C₁-C₄ alkylene, C₁-C₃ alkylene, C₁-C₂ alkylene, C₁ alkylene. Representative alkylene groups include, but are not limited to, methylene, ethylene, propylene, n-butylene, ethenylene, propenylene, n-butenylene, propynylene, n-butynylene, and the like. In some embodiments, Z is a linear or branched carbon chain of 1 to 30 carbon atoms or a linear or branched heteroatom-containing carbon chains of 1 to 30 atoms. In some embodiments, Z is a linear or branched carbon chain of 1 to 10 carbon atoms. For example, in some embodiments, Z is

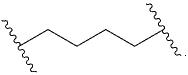 [SMILES: *CCCC*]

In some embodiments, Z is a branched carbon chain of 1 to 30 carbon atoms. In some embodiments, Z is a linear or branched heteroatom-containing carbon chain of 1 to 30 atoms. For example, Z may be a linear or branched carbon chain with one or more of the carbon atoms substituted with a heteroatom, including but not limited to O, N, S, or P. In some embodiments, is Z a carbocycle containing 3 to 30 carbon atoms. In some embodiments, Z is an alkylene-carbocycle containing 3 to 30 carbon atoms. For example, in some embodiments, Z is

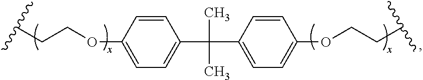

wherein x is 1-1000. In some embodiments, Z is a heterocycle containing 3 to 30 atoms. In some embodiments, Z is an alkylene-heterocycle containing 3 to 30 atoms. In some embodiments, Z is unsubstituted. In some embodiments, Z is substituted. In some embodiments, Z is one of the following

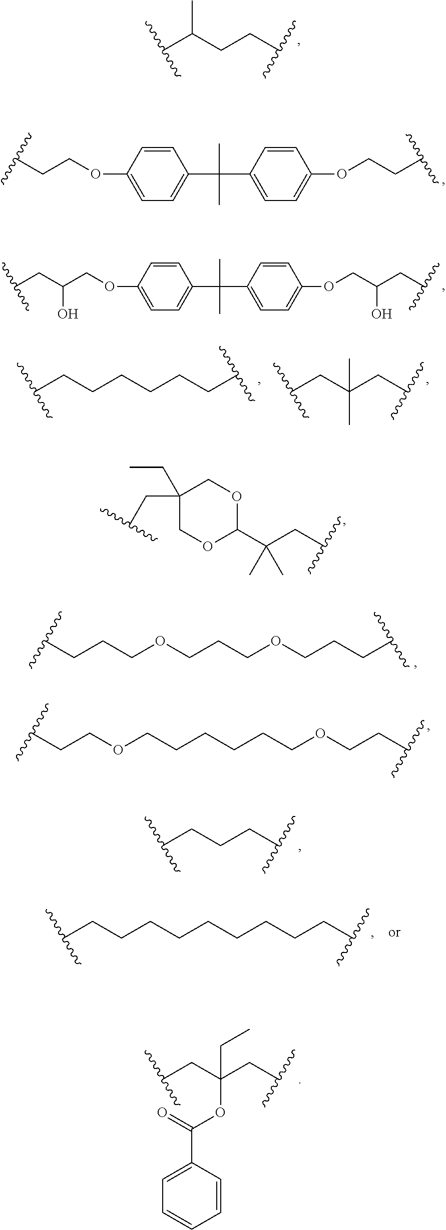

In some embodiments, Z′ is a linear or branched carbon chain of 1 to 30 carbon atoms, a linear or branched heteroatom-containing carbon chains of 1 to 30 atoms, a carbocycle containing 3 to 30 carbon atoms, an alkylene-carbocycle containing 3 to 30 carbon atoms, a heterocycle containing 3 to 30 atoms, or an alkylene-heterocycle containing 3 to 30 atoms. Z′ may be unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ether, a C₁-C₆ thioether, a C₁-C₆ sulfone, a C₁-C₆ sulfoxide, a C₁-C₆primary amide, a C₁-C₆ secondary amide, a halo C₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl and C₆-C₁₀ aryl; wherein each R′ is independently selected, from the group consisting of hydrogen and C₁-C₆ alkyl. In some embodiments, Z′ is a linear carbon chain of 1 to 30 carbon atoms. For example, Z′ may be an alkylene group including, but not limited to, C₁-C₂₄ alkylene, C₁-C₂₀ alkylene, C₁-C₁₆ alkylene, C₁-C₁₂ alkylene, C₁-C₈ alkylene, C₁-C₆ alkylene, C₁-C₄ alkylene, C₁-C₃ alkylene, C₁-C₂ alkylene, C₁ alkylene. Representative alkylene groups include, but are not limited to, methylene, ethylene, propylene, n-butylene, ethenylene, propenylene, n-butenylene, propynylene, n-butynylene, and the like. In some embodiments, Z′ is a linear or branched carbon chain of 1 to 30 carbon atoms or a linear or branched heteroatom-containing carbon chains of 1 to 30 atoms.

In some embodiments, Z″ is a linear or branched carbon chain of 1 to 30 carbon atoms, a linear or branched heteroatom-containing carbon chains of 1 to 30 atoms, a carbocycle containing 3 to 30 carbon atoms, an alkylene-carbocycle containing 3 to 30 carbon atoms, a heterocycle containing 3 to 30 atoms, or an alkylene-heterocycle containing 3 to 30 atoms. Z″ may be unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ether, a C₁-C₆ thioether, a C₁-C₆ sulfone, a C₁-C₆ sulfoxide, a C₁-C₆primary amide, a C₁-C₆ secondary amide, a halo C₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl and C₆-C₁₀ aryl; wherein each R′ is independently selected, from the group consisting of hydrogen and C₁-C₆ alkyl. In some embodiments, Z″ is a linear carbon chain of 1 to 30 carbon atoms. For example, Z″ may be an alkylene group including, but not limited to, C₁-C₂₄ alkylene, C₁-C₂₀ alkylene, C₁-C₁₆ alkylene, C₁-C₁₂ alkylene, C₁-C₈ alkylene, C₁-C₆ alkylene, C₁-C₄ alkylene, C₁-C₃ alkylene, C₁-C₂ alkylene, C₁ alkylene. Representative alkylene groups include, but are not limited to, methylene, ethylene, propylene, n-butylene, ethenylene, propenylene, n-butenylene, propynylene, n-butynylene, and the like. In some embodiments, Z″ is a linear or branched carbon chain of 1 to 30 carbon atoms or a linear or branched heteroatom-containing carbon chains of 1 to 30 atoms.

In accordance with certain embodiments of the present disclosure, G may be —C—, —S—, —S(O)—, —P(OR₁)—, or —P(OH)—; thus forming a carbonyl, sulfoxide, sulfone, and phosphono group, respectively. Thus, in some embodiments, G is —C—. In some embodiments, G is —S—. In some embodiments, G is —S(O)—.

In some embodiments, the compound of formula (B) is

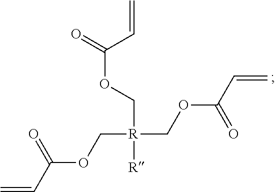

wherein

R is a linear or branched carbon chain of 1 to 10 carbon atoms, a linear or branched heteroatom-containing carbon chains of 1 to 10 atoms, a carbocycle containing 3 to 10 carbon atoms, or a heterocycle containing 3 to 10 atoms, and R is unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ ether, a C₁-C₆ thioether, a C₁-C₆ sulfone, a C₁-C₆ sulfoxide, a C₁-C₆ primary amide, a C₁-C₆ secondary amide, a halo C₁-C₆alkyl, a carboxyl group, a cyano group, a nitro group, a nitroso group, —OC(O)NR′R′, —N(R′)C(O)NR′R′, N(R′)C(O)O—C₁-C₆alkyl, C₃-C₆ cycloalkyl, C₃-C₆ heterocyclyl, C₂-C₅ heteroaryl and C₆-C₁₀ aryl; wherein each R′ is independently selected, from the group consisting of hydrogen and C₁-C₆ alkyl; and R″ is an unsubstituted or substituted, linear or branched carbon chain of 1 to 10 carbon atoms, a linear or branched heteroatom-containing carbon chains of 1 to 10 atoms, a carbocycle containing 3 to 10 carbon atoms, or a heterocycle containing 3 to 10 atoms. In some embodiments, R is 1 carbon atom. In some embodiments, R″ is a linear or branched carbon chain, such as methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, n-pentyl, isopentyl, neopentyl, n-hexyl. For example, in some embodiments, the compound of formula (B) is

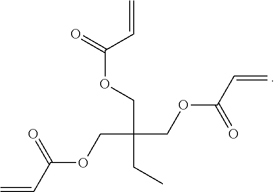 [SMILES: C=CC(=O)OCC(CC)(COC(=O)C=C)COC(=O)C=C]

In some embodiments, R is a carbocycle containing 3 to 10 carbon atoms. For example R may be cyclopropyl, cyclobutyl, cyclopentyl, cyclohexyl, cycloheptyl, cyclooctyl, cyclononyl, cyclodecyl, phenyl, or naphtyl. In some embodiments, R is a heterocycle containing 3 to 10 atoms.

In certain embodiments, the first amine has the formula R₁—NH₂ or R₁—N(H)—Z′—N(H)—R₁. In some embodiments, the first amine has the formula R₁—NH₂. In some embodiments, the first amine has the formula R₁—N(H)—Z′—N(H)—R₁. In some embodiments, the first amine having the formula R₁—N(H)—Z′—N(H)—R₁ is

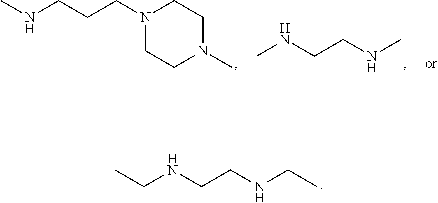

In some embodiments, the first amine has the formula R₁—N(H)—Z′—N—(R₁)₂. In some embodiments, the first amine having the formula R₁—N(H)—Z′—N—(R₁)₂ is

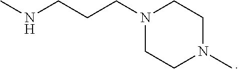

In certain embodiments, the second amine has the formula R₂—NH₂ or R₂—N(H)—Z″—N(H)—R₂. In some embodiments, the second amine has the formula R₂—NH₂. In some embodiments, the second amine has the formula R₂—N(H)—Z″—N(H)—R₂. In some embodiments, the second amine having the formula R₂—N(H)—Z″—N(H)—R₂ is

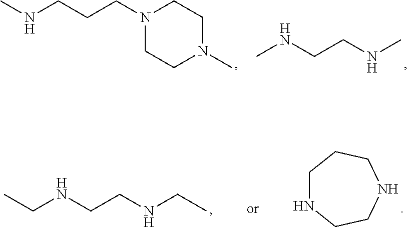

In some embodiments, the first amine has the formula R₂—N(H)—Z″—N—(R₂)₂. In some embodiments, the first amine having the formula R₂—N(H)—Z″—N—(R₂)₂ is

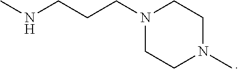

In certain embodiments, R₁ is C₁-C₄₀alkyl, C₁-C₄₀ heteroalkyl, C₂-C₄₀alkenyl, C₂-C₄₀ heteroalkenylene, C₄-C₈cycloalkenyl, C₂-C₄₀alkynyl, C₂-C₄₀ heteroalkynylene, C₃-C₈cycloalkyl, heterocyclyl, aryl, or heteroaryl; wherein the heterocyclyl and heteroaryl contain 1-5 heteroatoms selected from the group consisting of N, S, P and O; wherein the C₁-C₄₀alkyl, C₂-C₄₀alkenyl, C₄-C₈cycloalkenyl, C₂-C₄₀alkynyl, C₃-C₈cycloalkyl, heterocyclyl, aryl, and heteroaryl are optionally substituted with D, halogen, C₁-C₆alkyl, —OH, —NH(C₁-C₆alkyl), or —N(C₁-C₆alkyl)₂. R₁ may be unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ ether, a C₁-C₆ thioether, a C₁-C₆ sulfone, a C₁-C₆ sulfoxide, a C₁-C₆ primary amide, a C₁-C₆ secondary amide, a halo C₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl and C₆-C₁₀) aryl; wherein each R′ is independently selected, from the group consisting of hydrogen and C₁-C₆ alkyl. In some embodiments, R₁ is C₁-C₂₀ alkyl. For example, R₁ may be C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl groups such as such as methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, n-pentyl, isopentyl, neopentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, or n-icosyl. In some embodiments, R₁ is unsubstituted. In some embodiments, R₁ is substituted. In some embodiments, R₁ is selected from the group consisting of

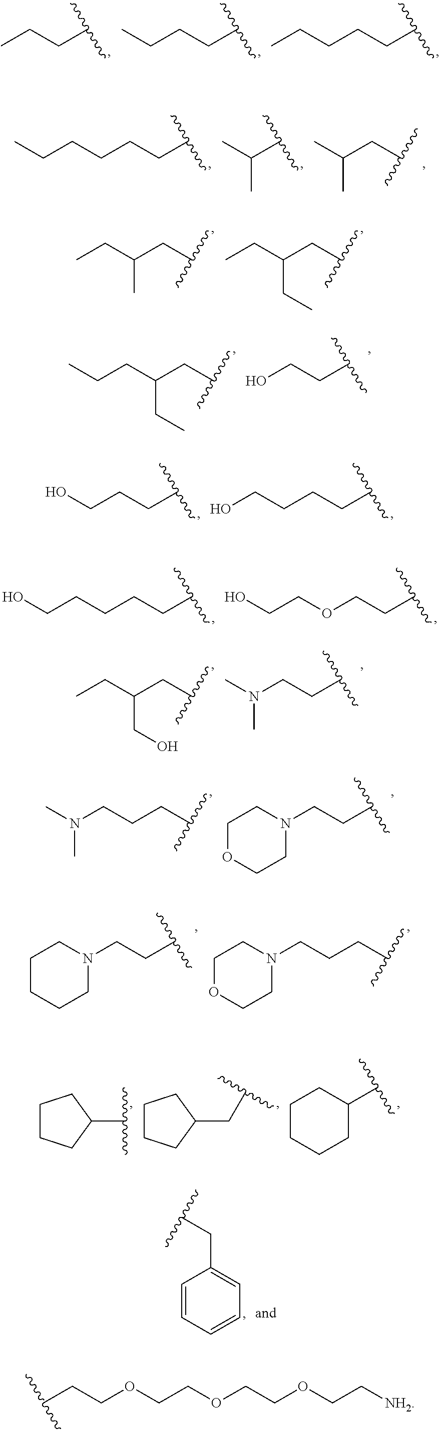

In some embodiments, R₁ is

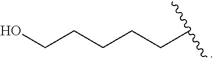 [SMILES: *CCCCCO]

In some embodiments, R₁ is

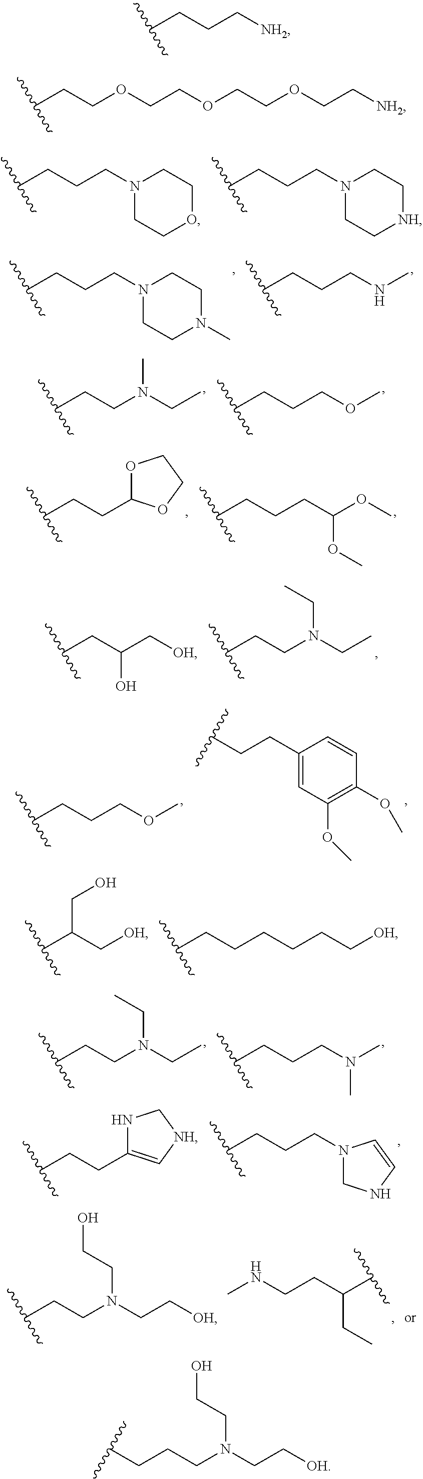

In certain embodiments, R₂ is C₁-C₄₀alkyl, C₁-C₄₀ heteroalkyl, C₂-C₄₀alkenyl, C₂-C₄₀ heteroalkenylene, C₄-C₈cycloalkenyl, C₂-C₄₀alkynyl, C₂-C₄₀ heteroalkynylene, C₃-C₈cycloalkyl, heterocyclyl, aryl, or heteroaryl; wherein the heterocyclyl and heteroaryl contain 1-5 heteroatoms selected from the group consisting of N, S, P and O; wherein the C₁-C₄₀alkyl, C₂-C₄₀alkenyl, C₄-C₈cycloalkenyl, C₂-C₄₀alkynyl, C₃-C₈cycloalkyl, heterocyclyl, aryl, and heteroaryl are optionally substituted with D, halogen, C₁-C₆alkyl, —OH, —O—C₁-C₆alkyl, —NH₂, —NH(C₁-C₆alkyl), or —N(C₁-C₆alkyl)₂. R₂ may be unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ ether, a C₁-C₆ thioether, a C₁-C₆ sulfone, a C₁-C₆ sulfoxide, a C₁-C₆ primary amide, a C₁-C₆ secondary amide, a halo C₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl and C₆-C₁₀) aryl; wherein each R′ is independently selected, from the group consisting of hydrogen and C₁-C₆ alkyl. In some embodiments, R₂ is C₁-C₂₀ alkyl. For example, R₂ may be C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl groups such as such as methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, n-pentyl, isopentyl, neopentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, or n-icosyl. In some embodiments, R₂ is unsubstituted. In some embodiments, R₂ is substituted. In some embodiments, R₂ is selected from the group consisting of

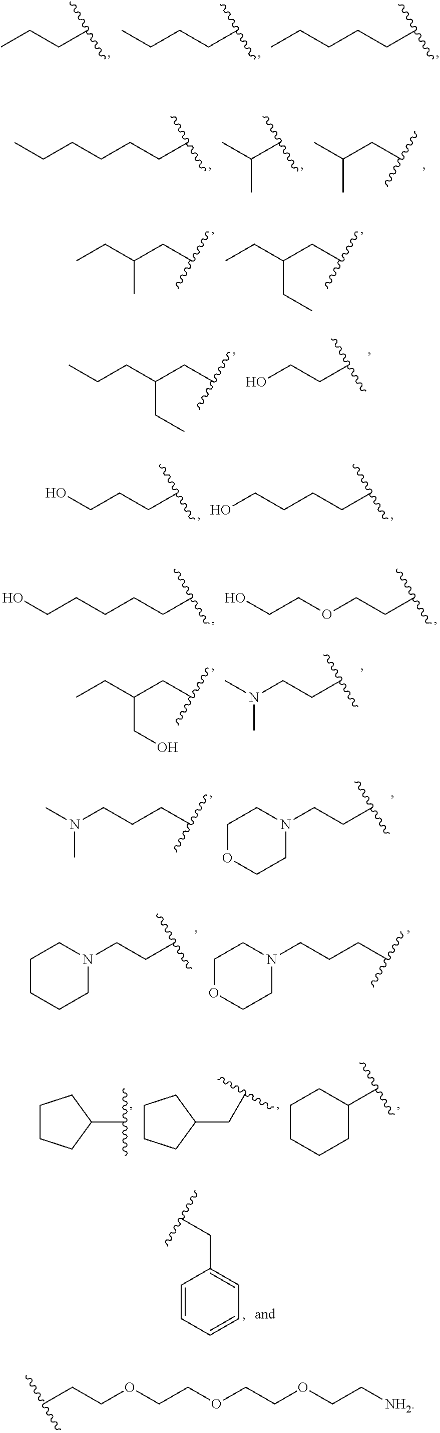

In some embodiments, R₂ is

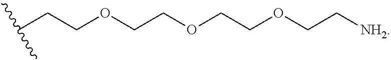 [SMILES: *CCOCCOCCOCCN]

In some embodiments, R₂ is

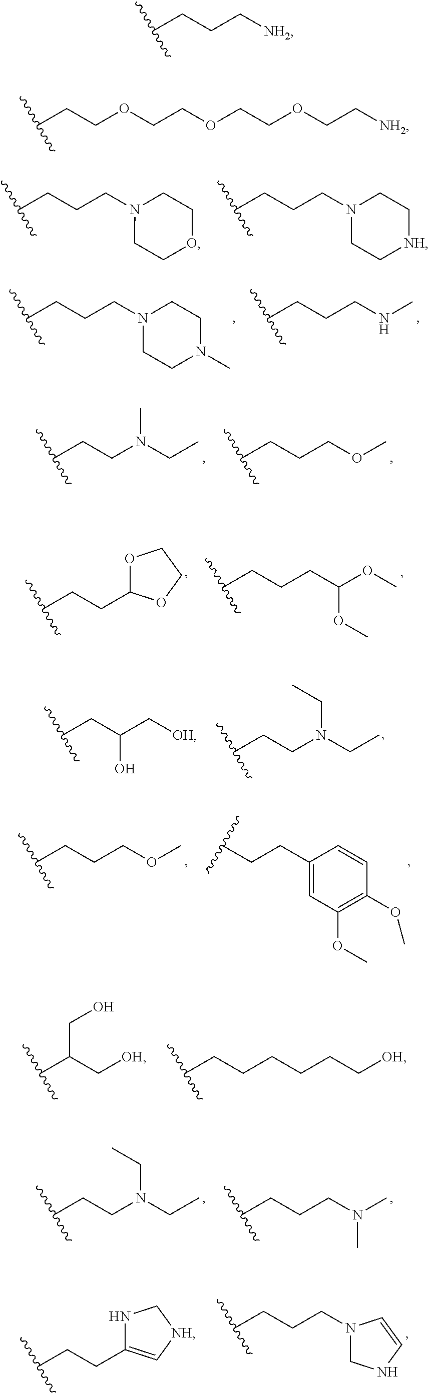

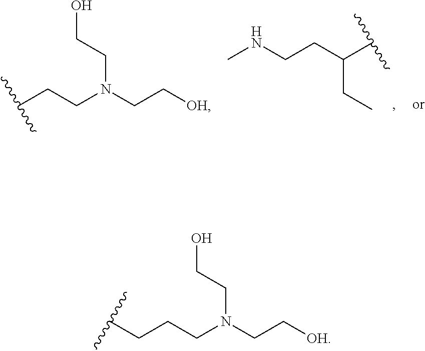

In some embodiments, each Q is H or a C₁-C₁₀ linear or branched alkyl group. Thus, in some embodiments, each Q is H. In other embodiments, each Q is a C₁-C₁₀ linear or branched alkyl group. For example, each Q may be methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, n-pentyl, isopentyl, neopentyl, n-hexyl, n-heptyl, n-octyl, n-nonyl, or n-decyl. In some embodiments, each Q is methyl.

In some embodiments, each J is —O—. In some embodiments, each J is —NH—.

In some embodiments, each E₁ is independently selected from the group consisting of covalent bond, —N—, —O—, —S—, alkylene, heteroalkylene, alkenyl, heteroalkenylene, alkynyl, heteroalkynylene. In some embodiments, each E₁ is heteroalkylene. In some embodiments, each E₁ is —CH₂—O—. In some embodiments, each n is at least 1. For example, n may be 1, 2, 3, 4, 5, 6, 7, 8, 9, or 10. In some embodiments, n is 1.

In some embodiments, A is a linear or branched carbon chain of 1 to 30 carbon atoms, a linear or branched heteroatom-containing carbon chains of 2 to 30 atoms, a carbocycle containing 3 to 30 carbon atoms, or a heterocycle containing 3 to 30 atoms. For example, in some embodiments, A is

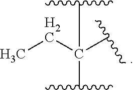 [SMILES: *C(*)(*)CC]

In some embodiments, the polymer of the present disclosure has the general structure of

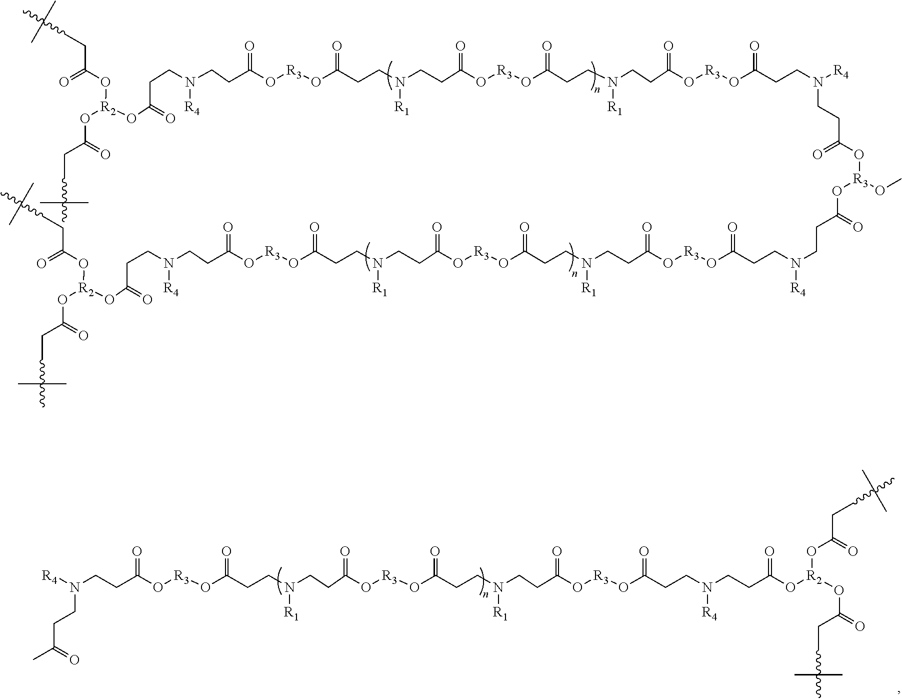

wherein the wavy bond represents a bond to rest of the polymer. Because of the highly controlled sequential linear oligomer growth and branching, the resulting polymers have a more uniform distribution of the linear segments and branching units, as illustrated in the above structure. As described in subsequent sections and examples, the polymers possess a strong DNA binding affinity and can condense DNA to formulate nanosized polyplexes with nearly 100% cellular uptake efficiency. In some embodiments, the polymer of the present disclosure is

In some embodiments, a molar excess of the compound of formula (A) is reacted with the first amine. For example, the stoichiometric ratio of the compound of formula (A) to the first amine may range from about 1.1:1 to about 10:1 including about 1.1:1, about 1.2:1, about 1.3:1, about 1.4:1, about 1.5:1, about 1.6:1, about 1.7:1, about 1.8:1, about 1.9:1, about 2:1, about 3:1, about 4:1, about 5:1, about 6:1, about 7:1, about 8:1, about 9:1 or about 10:1, including all ranges there between.

In some embodiments, the stoichiometric ratio of the compound of formula (A) to the first amine is about 1.1:1, about 1.2:1, about 1.3:1, about 1.4:1, about 1.5:1, about 1.6:1, about 1.7:1, about 1.8:1, about 1.9:1, about 2:1, about 3:1, about 4:1, about 5:1, about 6:1, about 7:1, about 8:1, about 9:1 or about 10:1. In some embodiments, the stoichiometric ratio of the compound of formula (A) to the first amine may range from about 1.1:1 to about 2:1. In some embodiments, the stoichiometric ratio the of the compound of formula (A) to the first amine is about 1.2:1. In some embodiments, the compound of formula (A) is reacted with the first amine at a molar equivalence (i.e. about 1:1).

In some embodiments, Step (a) is performed in an organic solvent. A wide variety of organic solvents can be used in the context of the present disclosure, including but not limited to dimethylsulfoxide (DMSO), N,N-dimethylformamide (DMF), N-methylpyrrolidone (NMP) and the like; ketones such as acetone, methyl ethyl ketone, methyl isobutyl ketone and the like; ethers such tetrahydrofuran (THF), diethylether, methyl tertiary-butyl ether and the like; hydrocarbons such as toluene, xylene, cyclohexane and the like. In some embodiments, Step (a) is performed in DMSO.

In some embodiments, Step (a) is performed at a temperature ranging from about 40° C. to about 120° C., including about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, or 120° C., including all ranges there between.

In some embodiments, Step (a) is performed at 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, or 120° C. In some embodiments, Step (a) is performed at about 90° C.

In some embodiments, the product of Step (a) is not purified before Step (b). In other embodiments, the product of Step (a) is purified before Step (b). The product of step (a) may be purified by a variety of methods and techniques apparent to a person having ordinary skill in the art.

In some embodiments, a molar excess of the second amine is added to the product of Step (a). For example, the stoichiometric ratio of the second amine to the product of Step (a) may range from about 1.1:1 to about 10:1 including about 1.1:1, about 1.2:1, about 1.3:1, about 1.4:1, about 1.5:1, about 1.6:1, about 1.7:1, about 1.8:1, about 1.9:1, about 2:1, about 3:1, about 4:1, about 5:1, about 6:1, about 7:1, about 8:1, about 9:1 or about 10:1, including all ranges there between.

In some embodiments, the stoichiometric ratio of the second amine to the product of Step (a) is about 1.1:1, about 1.2:1, about 1.3:1, about 1.4:1, about 1.5:1, about 1.6:1, about 1.7:1, about 1.8:1, about 1.9:1, about 2:1, about 3:1, about 4:1, about 5:1, about 6:1, about 7:1, about 8:1, about 9:1 or about 10:1. In some embodiments, the stoichiometric ratio of the of the second amine to the product of Step (a) is about 5:1. In some embodiments, the second amine is reacted with the product of Step (a) at a molar equivalence (i.e. about 1:1).

In some embodiments, Step (b) is performed at a temperature ranging from about 16° C. to about 40° C. For example, Step (b) is performed at a temperature ranging from about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, to about 40° C., including all ranges there between.

In some embodiments, Step (b) is performed at a temperature of about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about or about 40° C.

In some embodiments, the product of Step (b) is not purified before Step (c). In other embodiments, the product of Step (b) is purified before Step (c). The product of step (b) may be purified by a variety of methods and techniques apparent to a person having ordinary skill in the art. For example, the product of Step (b) may be purified by dialysis.

In some embodiments, Step (c) is performed at a temperature higher than that of Step (b). For example, Step (c) is performed at a temperature ranging from about 21° C. to about 200° C. For examples, Step (c) is performed at a temperature ranging from about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101 about, 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, to about 200° C., including all ranges there between.

In some embodiments, Step (c) is performed at about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101 about, 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, or about 200° C. In some embodiments, Step (c) is performed at about 90° C.

In some embodiments, the polymers made by methods of the present disclosure have an alpha parameter defined from the Mark-Houwink equation of less than about 0.5. For example, the polymers of the present disclosure have an alpha parameter defined from the Mark-Houwink equation ranging from about 0.01, about 0.02, about 0.03, about 0.04, about 0.05, about 0.06, about 0.07, about 0.08, about 0.09, about 0.10, about 0.11, about 0.12, about 0.13, about 0.14, about 0.15, about 0.16, about 0.17, about 0.18, about 0.19, about 0.20, about 0.21, about 0.22, about 0.23, about 0.24, about 0.25, about 0.26, about 0.27, about 0.28, about 0.29, about 0.30, about 0.31, about 0.32, about 0.33, about 0.34, about 0.35, about 0.36, about 0.37, about 0.38, about 0.39, about 0.40, about 0.41, about 0.42, about 0.43, about 0.44, about 0.45, about 0.46, about 0.47, about 0.48, about to about 0.49, including all ranges there between. In some embodiments, the polymers made by the methods of the present disclosure have an alpha parameter defined from the Mark-Houwink equation from about 0.2 to about 0.5.

In some embodiments, the polymers made by the methods of the present disclosure have an alpha parameter defined from the Mark-Houwink equation of about 0.01, about 0.02, about 0.03, about 0.04, about 0.05, about 0.06, about 0.07, about 0.08, about 0.09, about 0.10, about 0.11, about 0.12, about 0.13, about 0.14, about 0.15, about 0.16, about 0.17, about 0.18, about 0.19, about 0.20, about 0.21, about 0.22, about 0.23, about 0.24, about 0.25, about 0.26, about 0.27, about 0.28, about 0.29, about 0.30, about 0.31, about 0.32, about 0.33, about 0.34, about 0.35, about 0.36, about 0.37, about 0.38, about 0.39, about 0.40, about 0.41, about 0.42, about 0.43, about 0.44, about 0.45, about 0.46, about 0.47, about 0.48, or about 0.49.

In some embodiments, the polymers made by the methods of the present disclosure have a PDI from about 1.01 to about 8.0. For example, the PDI may range from about 1.01, about 1.02, about 1.03, about 1.04, about 1.05, about 1.06, about 1.07, about 1.08, about 1.09, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, to about 8.0, including all ranges there between.

In some embodiments, the polymers made by the methods of the present disclosure have a PDI of about 1.01, about 1.02, about 1.03, about 1.04, about 1.05, about 1.06, about 1.07, about 1.08, about 1.09, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. In some embodiments, the polymers of the present disclosure have a PDI of about 2.5. In some embodiments, the polymers of the present disclosure have a PDI of about 3.5. In some embodiments, the polymers of the present disclosure have a PDI of about 6.5. In some embodiments, the polymers of the present disclosure have a PDI of about 8.5.

In some embodiments, the polymers made by the methods of the present disclosure have a M_{W} of at least 3 kDa. In some embodiments, the polymers made by the methods of the present disclosure have a M_{W} of about 3 kDa to about 200 kDa. Accordingly, the polymers of the present disclosure have a M_{W} ranging from about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100 about, 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167 about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, to about 200 kDa. In some embodiments, the polymer has a M_{W} of between about 5 kDa and 50 kDa. In some embodiments, the polymer has a M_{W} of between about 10 kDa and 50 kDa.

In some embodiments, the polymers made by the methods of the present disclosure have a M_{W} about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100 about, 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167 about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, to about 200 kDa. In some embodiments, the polymer made by the method of the present disclosure has a M_{W} of between about 5 kDa and 50 kDa. In some embodiments, the polymer made by the method of the present disclosure has a M_{W} of about 10 kDa. In some embodiments, the polymer made by the method of the present disclosure has a M_{W} of about 20 kDa. In some embodiments, the polymer made by the method of the present disclosure has aMW of about 30 kDa. In some embodiments, the polymer made by the method of the present disclosure has a M_{W} of about 40 kDa.

In some embodiments, the product after Step (b) has a M_{W} of about 3 kDa.

### Polyplexes

In some embodiments, the present disclosure provides a polyplex comprising a nucleic acid component as described herein, and any of the branched polymers disclosed herein, for example a polymer made by any of the processes described herein or a polymer of formula (I):

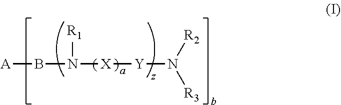

wherein

each A is independently a linear or branched carbon chain of 1 to 30 carbon atoms, a linear or branched heteroatom-containing carbon chains of 1 to 30 atoms, a carbocycle containing 3 to 30 carbon atoms, or a heterocycle containing 3 to 30 atoms;

wherein A is optionally substituted with one or more halogen, hydroxyl, amino group, sulfonyl group, sulphonamide group, thiol, C₁-C₆ alkyl, C₁-C₆ alkoxy, C₁-C₆ether, C₁-C₆ thioether, C₁-C₆ sulfone, C₁-C₆ sulfoxide, C₁-C₆primary amide, C₁-C₆ secondary amide, halo C₁-C₆ alkyl, carboxyl group, cyano group, nitro group, nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl or C₆-C₁₀ aryl; wherein each R′ is independently selected, from the group consisting of hydrogen and C₁-C₆ alkyl;

each B is independently a first linking moiety;

each X is independently

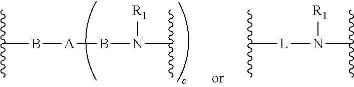

each Y is independently

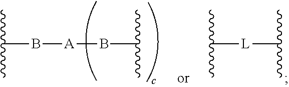

each L is independently a second linking moiety;

each R₁, R₂ and R₃ are independently, at each occurrence H, C₁-C₄₀alkyl, C₁-C₄₀ heteroalkyl, C₂-C₄₀alkenyl, C₂-C₄₀ heteroalkenylene, C₄-C₈cycloalkenyl, C₂-C₄₀alkynyl, C₂-C₄₀ heteroalkynylene, C₃-C₈cycloalkyl, heterocyclyl, aryl, or heteroaryl; wherein the heterocyclyl and heteroaryl contain 1-5 heteroatoms selected from the group consisting of N, S, P and O; wherein the C₁-C₆alkyl, C₂-C₈alkenyl, C₄-C₈cycloalkenyl, C₂-C₆alkynyl, C₃-C₈cycloalkyl, heterocyclyl, aryl, and heteroaryl are optionally substituted with D, halogen, C₁-C₆alkyl, —OH, —O—C₁-C₆alkyl, —NH₂, —NH(C₁-C₆alkyl), or —N(C₁-C₆alkyl)₂; or

wherein R₂ and R₃ together with the atom to which they are attached can form heterocyclyl or heteroaryl containing 1-3 heteroatoms selected from the group consisting of N, S, P and O;

a is 1-1000;

b is 1-4;

c is 1-3; and

z is 1-100;

with the proviso that at least one of R₂ and R₃ is not H.

In certain embodiments, the polyplex comprises a nucleic acid component and a polymer of formula (II):

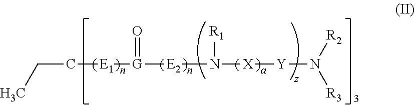

wherein,

each E₁ is selected from the group consisting of covalent bond, —N—, —O—, —S—, alkylene, heteroalkylene, alkenyl, heteroalkenylene, alkynyl, heteroalkynylene;

each E₂ is selected from the group consisting of covalent bond, —N—, —O—, —S—, alkylene, heteroalkylene, alkenyl, heteroalkenylene, alkynyl, heteroalkynylene;

G is —C—, —S—, —S(O)—, —P(OR₁)—, or —P(OH)—; and

n is at least 1.

In some embodiments, each B is independently

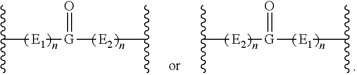

In some embodiments, each E₁ and E₂ are independently selected from the group consisting of covalent bond, —N—, —O—, —S—, alkylene, heteroalkylene, alkenyl, heteroalkenylene, alkynyl, heteroalkynylene. In some embodiments, each E₁ is heteroalkylene. In some embodiments, each E₁ is —CH₂—O—. In some embodiments, each E₂ is alkylene. In some embodiments, each E₂ is

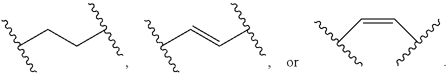

In some embodiments, each E₂ is

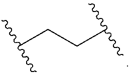 [SMILES: *CC*]

In some embodiments, each n is at least 1. For example, n may be 1, 2, 3, 4, 5, 6, 7, 8, 9, or 10. In some embodiments, n is 1. In some embodiments, G is —C—. In some embodiments, each B is

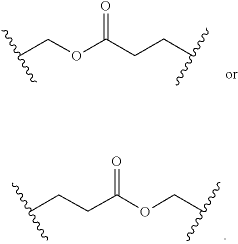 [SMILES: *CCC(=O)OC* ; *CCC(=O)OC*]

In some embodiments, B and A combine to form

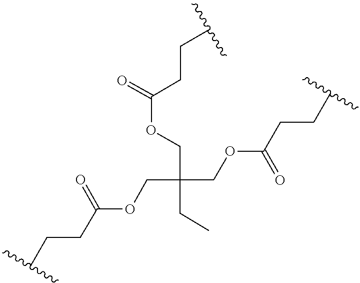 [SMILES: *CCC(=O)OCC(CC)(COC(=O)CC*)COC(=O)CC*]

In some embodiments, each L is

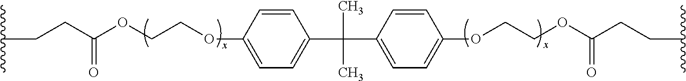

wherein x is 1-1000. In some embodiments, a is at least 2, b is 3, and each X is

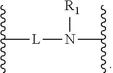

In some embodiments, each A is

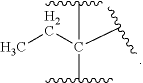 [SMILES: *C(*)(*)CC]

In some embodiments, each L is

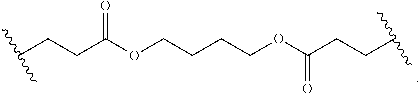 [SMILES: *CCC(=O)OCCCCOC(=O)CC*]

In some embodiments, Y is

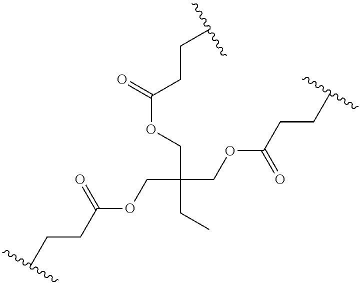 [SMILES: *CCC(=O)OCC(CC)(COC(=O)CC*)COC(=O)CC*]

In some embodiments, each R₂ and/or R₃ is

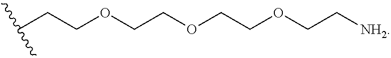 [SMILES: *CCOCCOCCOCCN]

In some embodiments, each R₁ is

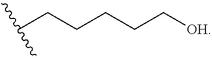 [SMILES: *CCCCCO]

In some embodiments, the polyplex comprises a nucleic acid component and a polymer of formulae (III) to (VIIe):

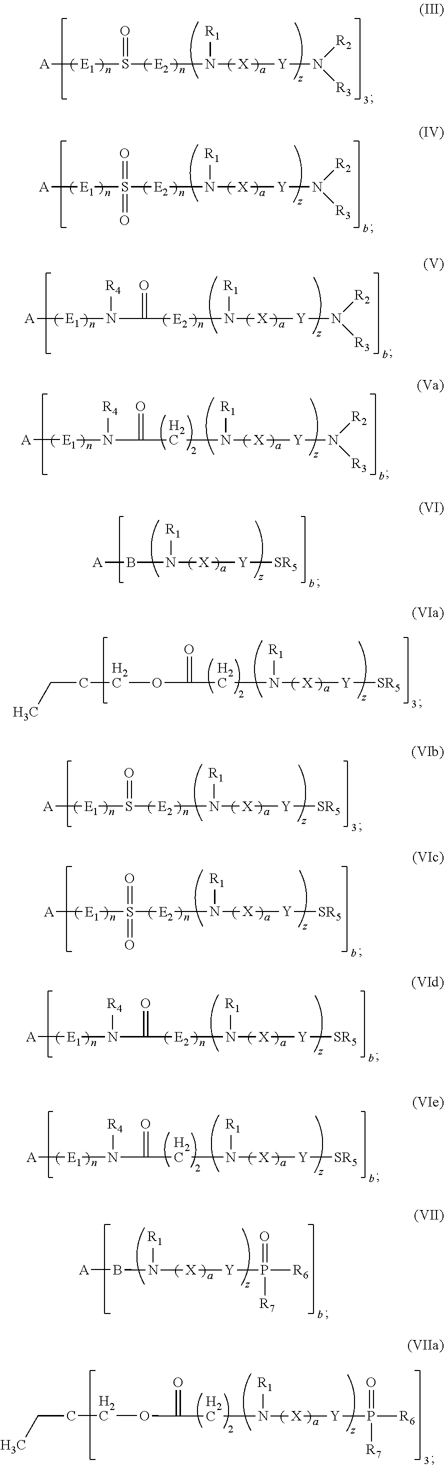

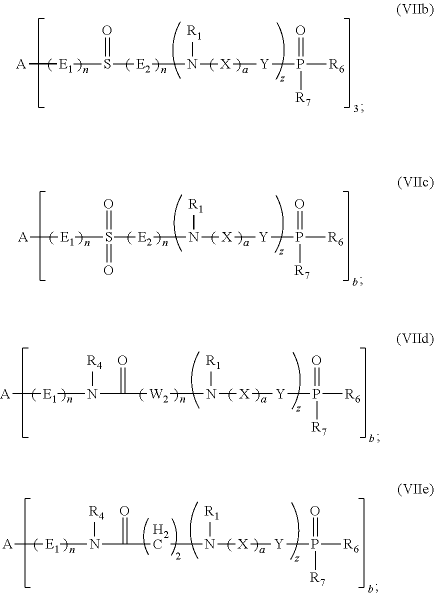

wherein each of R₅, R₆ and R₇ are independently, at each occurrence H, C₁-C₄₀alkyl, C₁-C₄₀ heteroalkyl, C₂-C₄₀alkenyl, C₂-C₄₀ heteroalkenylene, C₄-C₈cycloalkenyl, C₂-C₄₀alkynyl, C₂-C₄₀ heteroalkynylene, C₃-C₈cycloalkyl, heterocyclyl, aryl, or heteroaryl; wherein the heterocyclyl and heteroaryl contain 1-5 heteroatoms selected from the group consisting of N, S, P and O; wherein the C₁-C₆alkyl, C₂-C₈alkenyl, C₄-C₈cycloalkenyl, C₂-C₆alkynyl, C₃-C₈cycloalkyl, heterocyclyl, aryl, and heteroaryl are optionally substituted with D, halogen, C₁-C₆alkyl, —OH, —O—C₁-C₆alkyl, —NH₂, —NH(C₁-C₆alkyl), or —N(C₁-C₆alkyl)₂; and the remaining variables are as defined above.

In some embodiments, z is 1, 2, or 3. In some embodiments, z is 1.

In some embodiments, the polyplex comprises a nucleic acid component and a polymer comprising:

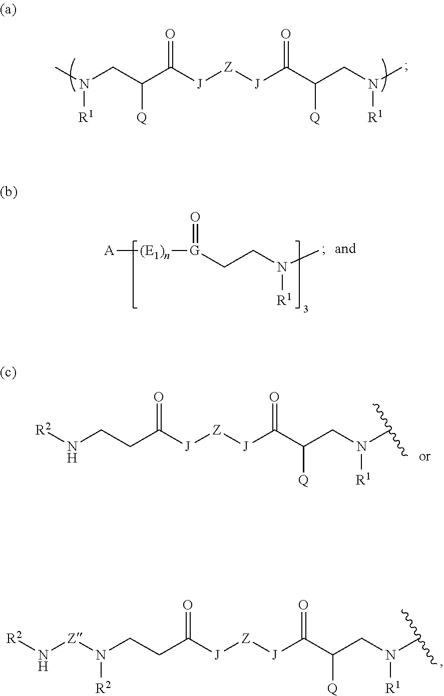

wherein R₁, R₂, A, E₁, G, J, Q, Z, Z″ and n have any of the definitions provided herein. In some further embodiments, the polymer has a M_{W} of about 3 kDa to about 200 kDa. In some further embodiments, the polymer has a M_{W} of about 5 kDa to about 50 kDa. In some further embodiments, the polymer has a M_{W} of between about 10 kDa and 50 kDa. In some further embodiments, the polymer has a M_{W} of about 5 kDa to about 15 kDa. In some further embodiments, the polymer has a M_{W} of about 10 kDa. In some further embodiments, the polymer has a M_{W} of about 20 kDa. In some further embodiments, the polymer has a M_{W} of about 30 kDa. In some further embodiments, the polymer has a M_{W} of about 40 kDa. In some further embodiments, the polymer has an alpha parameter defined from the Mark-Houwink of less than about 0.5. In some further embodiments, the polymer has an alpha parameter defined from the Mark-Houwink equation ranging from about 0.3 to about 0.5. In some further embodiments, the polymer has a PDI from about 1.0 to about 8.0. In some further embodiments, the polymer has a PDI of about 2.5.

In some embodiments, the polyplex comprises a nucleic acid component and a polymer comprising:

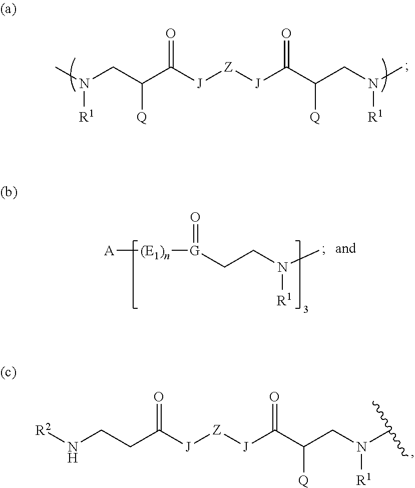

wherein R₁, R₂, A, E₁, G, J, Q, Z, and n have any of the definitions provided herein. In some further embodiments, the polymer has a M_{W} of about 3 kDa to about 200 kDa. In some further embodiments, the polymer has a M_{W} of about 5 kDa to about 50 kDa. In some further embodiments, the polymer has a M_{W} of between about 10 kDa and 50 kDa. In some further embodiments, the polymer has a M_{W} of about 5 kDa to about 15 kDa. In some further embodiments, the polymer has a M_{W} of about 10 kDa. In some further embodiments, the polymer has a M_{W} of about 20 kDa. In some further embodiments, the polymer has a M_{W} of about 30 kDa. In some further embodiments, the polymer has a M_{W} of about 40 kDa. In some further embodiments, the polymer has an alpha parameter defined from the Mark-Houwink of less than about 0.5. In some further embodiments, the polymer has an alpha parameter defined from the Mark-Houwink equation ranging from about 0.3 to about 0.5. In some further embodiments, the polymer has a PDI from about 1.0 to about 8.0. In some further embodiments, the polymer has a PDI of about 2.5.

In some embodiments, the polyplex comprises a nucleic acid component and a polymer comprising:

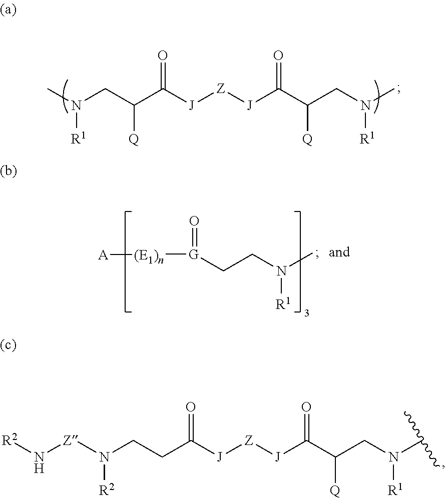

wherein R₁, R₂, A, E₁, G, J, Q, Z, Z″ and n have any of the definitions provided herein. In some further embodiments, the polymer has a M_{W} of about 3 kDa to about 200 kDa. In some further embodiments, the polymer has a M_{W} of about 5 kDa to about 50 kDa. In some further embodiments, the polymer has a M_{W} of between about 10 kDa and 50 kDa. In some further embodiments, the polymer has a M_{W} of about 5 kDa to about 15 kDa. In some further embodiments, the polymer has a M_{W} of about 10 kDa. In some further embodiments, the polymer has a M_{W} of about 20 kDa. In some further embodiments, the polymer has a M_{W} of about 30 kDa. In some further embodiments, the polymer has a M_{W} of about 40 kDa. In some further embodiments, the polymer has an alpha parameter defined from the Mark-Houwink of less than about 0.5. In some further embodiments, the polymer has an alpha parameter defined from the Mark-Houwink equation ranging from about 0.3 to about 0.5. In some further embodiments, the polymer has a PDI from about 1.0 to about 8.0. In some further embodiments, the polymer has a PDI of about 2.5.

In some further embodiments, the polyplex comprises a nucleic acid component and a polymer comprising:

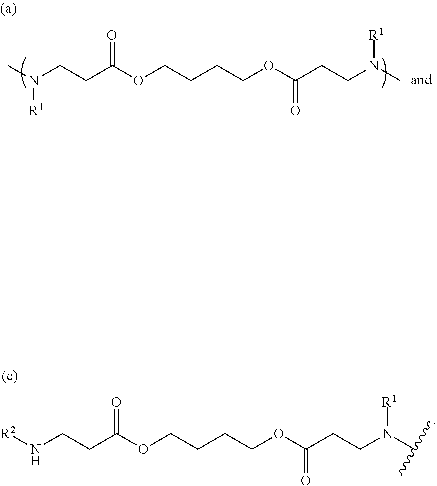

In some further embodiments, the polyplex comprises a nucleic acid component and a polymer comprising:

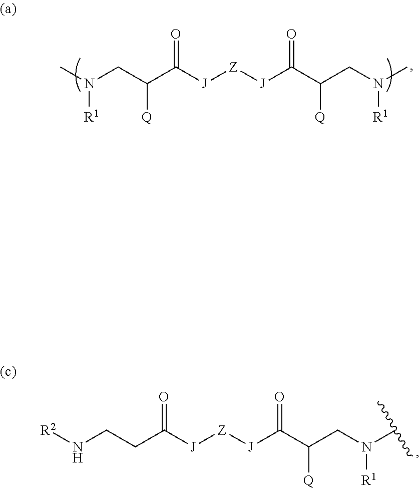

and
wherein

J is O and Z is

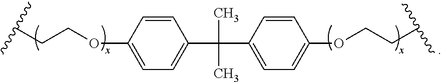

wherein x is 1-1000.

In some further embodiments, the polyplex comprises a nucleic acid component and a polymer comprising:

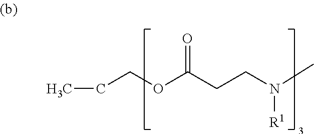

In some further embodiments, the polyplex comprises a nucleic acid component and a polymer, wherein R₁ is selected from

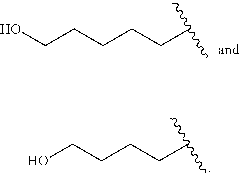 [SMILES: *CCCCCO ; *CCCCO]

In some further embodiments, the polyplex comprises a nucleic acid component and a polymer, wherein R₁ is

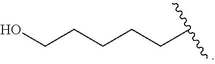 [SMILES: *CCCCCO]

In some further embodiments, the polyplex comprises a nucleic acid component and a polymer, wherein R₁ is

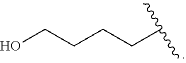 [SMILES: *CCCCO]

In some further embodiments, the polyplex comprises a nucleic acid component and a polymer, wherein R₂ is selected from

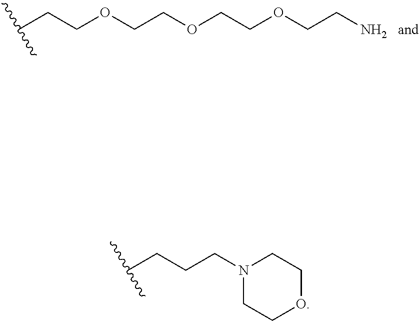 [SMILES: *CCOCCOCCOCCN ; *CCCN1CCOCC1]

In some further embodiments, the polyplex comprises a nucleic acid component and a polymer, wherein R₂ is

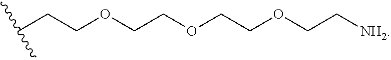 [SMILES: *CCOCCOCCOCCN]

In some further embodiments the polyplex comprises a nucleic acid component and a polymer, wherein R₂ is

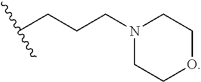 [SMILES: *CCCN1CCOCC1]

In some further embodiments, the polyplex comprises a nucleic acid component and a polymer, wherein R₁ is

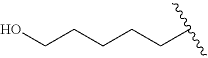 [SMILES: *CCCCCO]

### and R₂ is

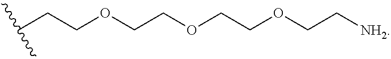 [SMILES: *CCOCCOCCOCCN]

In some further embodiments, the polyplex comprises a nucleic acid component and a polymer, wherein R₁ is

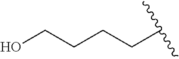 [SMILES: *CCCCO]

### and R₂ is

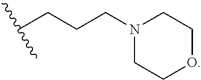 [SMILES: *CCCN1CCOCC1]

In some embodiments, the polyplex comprises a nucleic acid component and a polymer comprising:

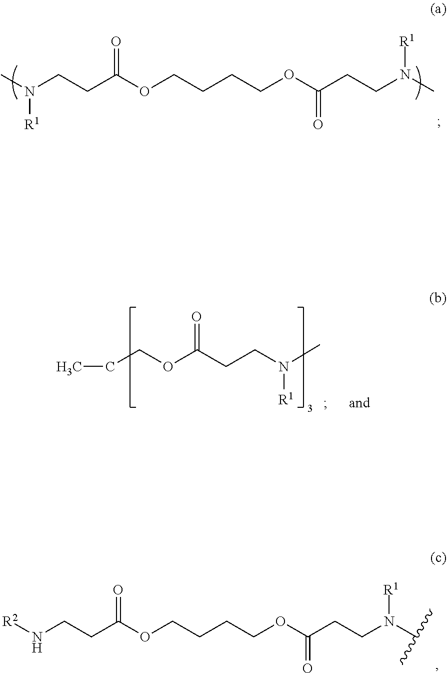

wherein

### R₁ is

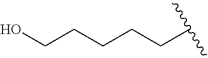 [SMILES: *CCCCCO]

and
R₂ is selected from

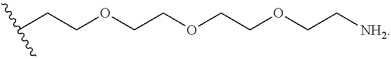 [SMILES: *CCOCCOCCOCCN]

In some further embodiments, the polymer has a M_{W} of about 3 kDa to about 200 kDa. In some further embodiments, the polymer has a M_{W} of about 5 kDa to about 50 kDa. In some further embodiments, the polymer has a M_{W} of between about 10 kDa and 50 kDa. In some further embodiments, the polymer has a M_{W} of about 5 kDa to about 15 kDa. In some further embodiments, the polymer has a M_{W} of about 10 kDa. In some further embodiments, the polymer has a M_{W} of about 20 kDa. In some further embodiments, the polymer has a M_{W} of about 30 kDa. In some further embodiments, the polymer has a M_{W} of about 40 kDa. In some further embodiments, the polymer has an alpha parameter defined from the Mark-Houwink of less than about 0.5. In some further embodiments, the polymer has an alpha parameter defined from the Mark-Houwink equation ranging from about 0.3 to about 0.5. In some further embodiments, the polymer has a PDI from about 1.0 to about 8.0. In some further embodiments, the polymer has a PDI of about 2.5.

In some embodiments, the polyplex comprises a nucleic acid component and a polymer comprising:

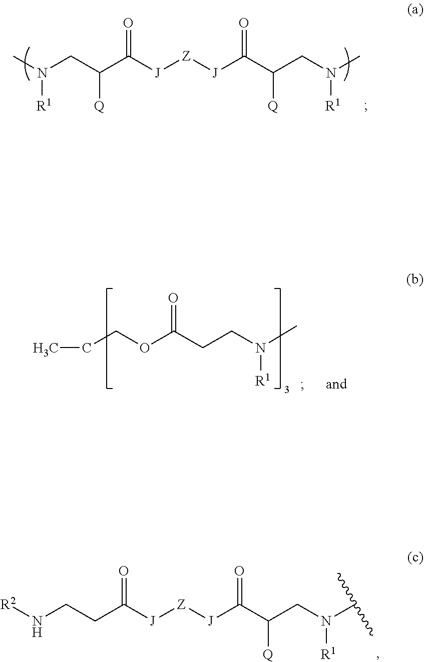

wherein

J is O and Z is

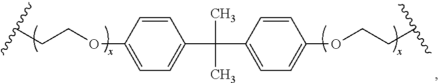

wherein x is 1-1000;

R₁ is

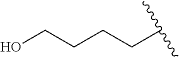 [SMILES: *CCCCO]

and

R₂ is

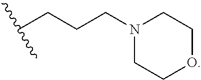 [SMILES: *CCCN1CCOCC1]

In some further embodiments, the polymer has a M_{W} of about 3 kDa to about 200 kDa. In some further embodiments, the polymer has a M_{W} of about 5 kDa to about 50 kDa. In some further embodiments, the polymer has a M_{W} of between about 10 kDa and 50 kDa. In some further embodiments, the polymer has a M_{W} of about 5 kDa to about 15 kDa. In some further embodiments, the polymer has a M_{W} of about 10 kDa. In some further embodiments, the polymer has a M_{W} of about 20 kDa. In some further embodiments, the polymer has a M_{W} of about 30 kDa. In some further embodiments, the polymer has a M_{W} of about 40 kDa. In some further embodiments, the polymer has an alpha parameter defined from the Mark-Houwink of less than about 0.5. In some further embodiments, the polymer has an alpha parameter defined from the Mark-Houwink equation ranging from about 0.3 to about 0.5. In some further embodiments, the polymer has a PDI from about 1.0 to about 8.0. In some further embodiments, the polymer has a PDI of about 2.5.

In some embodiments, the polymer and nucleic acid component are present at a ratio of from about 0.1:1 to about 200:1 (w/w). For example, the polymer and nucleic acid component are present at a ratio ranging from about 0.1:1, about 0.2:1, about 0.3:1, about 0.4:1, about 0.5:1, about 0.6:1, about 0.7:1, about 0.8:1, about 0.9:1, about 1:1, about 2:1, about 3:1, about 4:1, about 5:1, about 6:1, about 7:1, about 8:1, about 9:1, about 10:1 about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 37:1, about 38:1, about 39:1, about 40:1, about 41:1, about 42:1, about 43:1, about 44:1, about 45:1, about 46:1, about 47:1, about 48:1, about 49:1, about 50:1, about 51:1, about 52:1, about 53:1, about 54:1, about 55:1, about 56:1, about 57:1, about 58:1, about 59:1, about 60:1, about 61:1, about 62:1, about 63:1, about 64:1, about 65:1, about 66:1, about 67:1, about 68:1, about 69:1, about 70:1, about 71:1, about 72:1, about 73:1, about 74:1, about 75:1, about 76:1, about 77:1, about 78:1, about 79:1, about 80:1, about 81:1, about 82:1, about 83:1, about 84:1, about 85:1, about 86:1, about 87:1, about 88:1, about 89:1, about 90:1, about 91:1, about 92:1, about 93:1, about 94:1, about 95:1, about 96:1, about 97:1, about 98:1, about 99:1, about 100:1 about 101:1, about 102:1, about 103:1, about 104:1, about 105:1, about 106:1, about 107:1, about 108:1, about 109:1, about 110:1, about 111:1, about 112:1, about 113:1, about 114:1, about 115:1, about 116:1, about 117:1, about 118:1, about 119:1, about 120:1, about 121:1, about 122:1, about 123:1, about 124:1, about 125:1, about 126:1, about 127:1, about 128:1, about 129:1, about 130:1, about 131:1, about 132:1, about 133:1, about 134:1, about 135:1, about 136:1, about 137:1, about 138:1, about 139:1, about 140:1, about 141:1, about 142:1, about 143:1, about 144:1, about 145:1, about 146:1, about 147:1, about 148:1, about 149:1, about 150:1, about 151:1, about 152:1, about 153:1, about 154:1, about 155:1, about 156:1, about 157:1, about 158:1, about 159:1, about 160:1, about 161:1, about 162:1, about 163:1, about 164:1, about 165:1, about 166:1, about 167:1, about 168:1, about 169:1, about 170:1, about 171:1, about 172:1, about 173:1, about 174:1, about 175:1, about 176:1, about 177:1, about 178:1, about 179:1, about 180:1, about 181:1, about 182:1, about 183:1, about 184:1, about 185:1, about 186:1, about 187:1, about 188:1, about 189:1, about 190:1, about 191:1, about 192:1, about 193:1, about 194:1, about 195:1, about 196:1, about 197:1, about 198:1, about 199:1 to about 200:1, including all ranges there between. In some embodiments, the polymer and nucleic acid component are present at a ratio of from about 20:1 to about 80:1 (w/w).

In some embodiments, the polymer and nucleic acid component are present at a ratio of about 0.1:1, about 0.2:1, about 0.3:1, about 0.4:1, about 0.5:1, about 0.6:1, about 0.7:1, about 0.8:1, about 0.9:1, about 1:1, about 2:1, about 3:1, about 4:1, about 5:1, about 6:1, about 7:1, about 8:1, about 9:1, about 10:1 about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 37:1, about 38:1, about 39:1, about 40:1, about 41:1, about 42:1, about 43:1, about 44:1, about 45:1, about 46:1, about 47:1, about 48:1, about 49:1, about 50:1, about 51:1, about 52:1, about 53:1, about 54:1, about 55:1, about 56:1, about 57:1, about 58:1, about 59:1, about 60:1, about 61:1, about 62:1, about 63:1, about 64:1, about 65:1, about 66:1, about 67:1, about 68:1, about 69:1, about 70:1, about 71:1, about 72:1, about 73:1, about 74:1, about 75:1, about 76:1, about 77:1, about 78:1, about 79:1, about 80:1, about 81:1, about 82:1, about 83:1, about 84:1, about 85:1, about 86:1, about 87:1, about 88:1, about 89:1, about 90:1, about 91:1, about 92:1, about 93:1, about 94:1, about 95:1, about 96:1, about 97:1, about 98:1, about 99:1, about 100:1 about 101:1, about 102:1, about 103:1, about 104:1, about 105:1, about 106:1, about 107:1, about 108:1, about 109:1, about 110:1, about 111:1, about 112:1, about 113:1, about 114:1, about 115:1, about 116:1, about 117:1, about 118:1, about 119:1, about 120:1, about 121:1, about 122:1, about 123:1, about 124:1, about 125:1, about 126:1, about 127:1, about 128:1, about 129:1, about 130:1, about 131:1, about 132:1, about 133:1, about 134:1, about 135:1, about 136:1, about 137:1, about 138:1, about 139:1, about 140:1, about 141:1, about 142:1, about 143:1, about 144:1, about 145:1, about 146:1, about 147:1, about 148:1, about 149:1, about 150:1, about 151:1, about 152:1, about 153:1, about 154:1, about 155:1, about 156:1, about 157:1, about 158:1, about 159:1, about 160:1, about 161:1, about 162:1, about 163:1, about 164:1, about 165:1, about 166:1, about 167:1, about 168:1, about 169:1, about 170:1, about 171:1, about 172:1, about 173:1, about 174:1, about 175:1, about 176:1, about 177:1, about 178:1, about 179:1, about 180:1, about 181:1, about 182:1, about 183:1, about 184:1, about 185:1, about 186:1, about 187:1, about 188:1, about 189:1, about 190:1, about 191:1, about 192:1, about 193:1, about 194:1, about 195:1, about 196:1, about 197:1, about 198:1, about 199:1 or about 200:1. In some embodiments, the polymer and nucleic acid component are present at a ratio of about 30:1 (w/w).

In some embodiments, the particle size is less than 2 μm. In some embodiments, the particle size of the polyplex is less than about 300 nm. For example, the particle size of the polyplex may be about 50, 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, about 200, about 201, about 202, about 203, about 204, about 205, about 206, about 207, about 208, about 209, about 210, about 211, about 212, about 213, about 214, about 215, about 216, about 217, about 218, about 219, about 220, about 221, about 222, about 223, about 224, about 225, about 226, about 227, about 228, about 229, about 230, about 231, about 232, about 233, about 234, about 235, about 236, about 237, about 238, about 239, about 240, about 241, about 242, about 243, about 244, about 245, about 246, about 247, about 248, about 249, about 250, about 251, about 252, about 253, about 254, about 255, about 256, about 257, about 258, about 259, about 260, about 261, about 262, about 263, about 264, about 265, about 266, about 267, about 268, about 269, about 270, about 271, about 272, about 273, about 274, about 275, about 276, about 277, about 278, about 279, about 280, about 281, about 282, about 283, about 284, about 285, about 286, about 287, about 288, about 289, about 290, about 291, about 292, about 293, about 294, about 295, about 296, about 297, about 298, about 299, or about 300 nm. In some embodiments, the polyplexes of the present disclosure have a particle size of about 60 nm to about 250 nm. In some embodiments, the polyplexes of the present disclosure have a particle size of about 175 nm to about 250 nm.

In some embodiments, the polyplexes of the present disclosure have a zeta potential from about 0 mV to about 100 mV. For example, the polyplexes of the present disclosure may have a zeta potential ranging from about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 78, about 79, about 80, about 81, about 82, about 83, about 84, about 85, about 86, about 87, about 88, about 89, about 90, about 91, about 92, about 93, about 94, about 95, about 96, about 97, about 98, about 99 to about 100 mV. In some embodiments, the zeta potential is from about 30 mV to about 34 mV.

In some embodiments, the polyplexes of the present disclosure have a zeta potential of about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 78, about 79, about 80, about 81, about 82, about 83, about 84, about 85, about 86, about 87, about 88, about 89, about 90, about 91, about 92, about 93, about 94, about 95, about 96, about 97, about 98, about 99 or about 100 mV.

In some embodiments, the nucleic acid component of the polyplex is a plasmid, nanoplasmid, nucleic acid, minicircle, or gene editing system. In some embodiments, the nucleic acid component of the polyplex is a plasmid. In some embodiments, the nucleic acid component of the polyplex is a nanoplasmid. In some embodiments, the nanoplasmid comprises a eukaryotic transgene and a bacterial backbone that is less than 0.5 kb in size. In some embodiments, the plasmid or nanoplasmid is an antibiotic resistance marker-free plasmid or antibiotic resistance marker-free nanoplasmid. In some embodiments, the plasmid or nanoplasmid comprises a sucrose selection marker or nonsense suppressor marker.

In some embodiments, the nucleic acid component of the polyplex is a gene editing system. In some embodiments, the gene editing system is a (i) clustered, regularly interspaced, palindromic repeats (CRISPR)-associated (Cas) system; (ii) a transcription activator-like effector nuclease (TALEN) system; or (iii) a zinc finger nuclease (ZFN) system.

In some embodiments, the nucleic acid is an RNAi-inducing molecule. The RNAi-inducing molecule may be selected from the group consisting of siRNA, dsRNA, shRNA, and microRNA.

In some embodiments, the nucleic acid component comprises a tissue-specific promoter.

In some embodiments, the nucleic acid component comprises a gene associated with a genetic disease or disorder. The genetic disease or disorder may be caused by a mutation in one or more genes that results in low, absent, or dysfunctional protein expression. The gene may be selected from the group consisting of COL7A1, LAMB3, ADA, SERPINA1, CFTR, HTT, NF1, PHA, HBS, FERMT1, KRT14, DSP, SPINK5, and FLG. In some embodiments, the gene is COL7A1 and the genetic disease or disorder is a form of epidermolysis bullosa. Epidermolysis bullosa includes Epidermolysis bullosa dystrophica (autosomal recessive), Epidermolysis bullosa dystrophica (localisata variant), Epidermolysis bullosa pruriginosa, Epidermolysis bullosa (pretibial), Epidermolysis bullosa simplex (Dowling-Meara-type), Epidermolysis bullosa simplex (Koebner-type), Epidermolysis bullosa simplex (recessive 1), Epidermolysis bullosa simplex (Weber-Cockayne-type), Epidermolysis bullosa (lethal acantholytic). In some embodiments, the genetic disorder or genetic disease is adenosine deaminase (ADA) deficiency, Alpha-1 Antitrypsin Deficiency, cystic fibrosis, Huntington's Disease, Neurofibromatosis Type 1, Phenylketonuria, Sickle Cell Disease, Sporadic Inclusion Body Myositis, Duchenne muscular dystrophy, Kindler syndrome, Junctional Epidermolysis Bullosa, Dermatopathia pigmentosa reticularis, Naegeli-Franceschetti-Jadassohn syndrome, Netherton Syndrome, Ichthyosis Vulgaris, Atopic Dermatitis, Usher's syndrome, Ehlers-Danlos syndrome, Homozygous Familial Hypercholesterolemia (HoFH), or Crohn's disease.

In some embodiments, the sequence of the gene is optimized for maximum protein expression upon delivery of the polyplex to a cell.

### Pharmaceutical Compositions

In some embodiments, the present disclosure provides a pharmaceutical composition comprising an effective amount of one or more polyplexes in accordance with certain embodiments of present disclosure, in combination with a pharmaceutically acceptable carrier.

In some embodiments, the present disclosure provides a pharmaceutical composition comprising an effective amount of one or more polyplexes described herein, in combination with a pharmaceutically acceptable excipient. In some embodiments, the pharmaceutically acceptable excipient is selected from the group consisting of one or more bulking agents, buffering agents, tonicity agents and cryoprotectants. In some embodiments, the bulking agent is selected from the group consisting of hydroxyethyl starch, trehalose, mannitol, lactose, and glycine. In some embodiments, the buffering agent is selected from the group consisting of a phosphate buffer, a tris HCl buffer, a citrate buffer, and histidine. In some embodiments, the tonicity agent is selected from the group consisting of mannitol, sucrose, glycine, glycerol, and sodium chloride.

In some embodiments, the present disclosure provides a pharmaceutical composition comprising an effective amount of one or more polyplexes described herein, in combination with a cryoprotectant. In some embodiments, the cryoprotectant is selected from the group consisting of glucose, sucrose, trehalose, lactose, mannitol, sorbitol, aerosil (colloidal silicon dioxide), maltose, poly(vinyl pyrrolidone), fructose, dextran, glycerol, poly(vinyl alcohol), glycine, hydroxypropyl-β-cyclodextrin, and gelatin. In certain embodiments, the cryoprotectant is selected from the group consisting of trehalose, sucrose, glucose and mannitol. In some embodiments, the cryoprotectant is sucrose.

In some embodiments, the pharmaceutically acceptable carrier is suitable for oral, parenteral, inhalation, topical, subcutaneous, intramuscular, intravenous, intraocular, or intradermal administration. In some embodiments, the pharmaceutical composition is formulated as a lotion selected from the group consisting of non-aqueous lotion, water-in-oil lotion, and oil-in-water lotion. In some embodiments, the pharmaceutical composition is lyophilized for future use. In some embodiments, the pharmaceutical composition is frozen in an aqueous solution.

In some embodiments, the pharmaceutical composition is a lyophil. In some embodiments, the lyophil comprises an effective amount of one or more polyplexes described herein, in combination with a pharmaceutically acceptable excipient. In certain embodiments, the pharmaceutically acceptable excipient comprises a cryoprotectant. In certain embodiments, the cryoprotectant is selected from the group consisting of trehalose, sucrose, glucose and mannitol. In some embodiments, the cryoprotectant is sucrose.

In some embodiments, the present disclosure provides methods of making pharmaceutical compositions comprising an effective amount of one or more polyplexes described herein in combination with a pharmaceutically acceptable carrier. In some embodiments, the method comprises combining one or more polyplexes described herein with a suitable solvent. In some embodiments, the suitable solvent is selected from the group consisting of water, dimethylsulfoxide and mixtures thereof. In certain embodiments, the suitable solvent comprises water.

In some embodiments, the method comprises:
(a) combining one or more polyplexes described herein with a suitable solvent;
(b) adding one or more pharmaceutically acceptable excipients to the mixture of Step (a) and
(c) lyophilizing the mixture of Step (b) to provide a lyophil.

In some embodiments, the one or more pharmaceutically acceptable excipient of step (b) comprises a cryoprotectant. In certain embodiments, the concentration of the cryoprotectant is from about 1% to about 20%, including about 2%, about 3%, about 4%, about 5%, about 6%, about 7%, about 8%, about 9%, about 10%, about 11%, about 12%, about 13%, about 14%, about 15%, about 16%, about 17%, about 18%, and about 19%, including all ranges therebetween, by weight of the Step (b) mixture. In certain embodiments, the concentration of the cryoprotectant is about 1% about 2%, about 3%, about 4%, about 5%, about 6%, about 7%, about 8%, about 9%, about 10%, about 11%, about 12%, about 13%, about 14%, about 15%, about 16%, about 17%, about 18%, about 19% or about 20% by weight of the Step (b) mixture. In particular embodiments, the concentration of the cryoprotectant is about 1% by weight of the Step (b) mixture. In particular embodiments, the concentration of the cryoprotectant is about 3% by weight of the Step (b) mixture. In particular embodiments, the concentration of the cryoprotectant is about 5% by weight of the Step (b) mixture.

In some embodiments, the present disclosure provides pharmaceutical compositions prepared according to the methods described herein.

In some embodiments, the present disclosure provides pharmaceutical compositions prepared by a method comprising:
(a) combining one or more polyplexes described herein with a suitable solvent;
(b) adding one or more pharmaceutically acceptable excipients to the mixture of Step (a) and
(c) lyophilizing the mixture of Step (b) to provide a lyophil.

In some embodiments, the one or more pharmaceutically acceptable excipient of Step (b) comprises a cryoprotectant. In certain embodiments, the concentration of the cryoprotectant is from about 1% to about 20%, including about 2%, about 3%, about 4%, about 5%, about 6%, about 7%, about 8%, about 9%, about 10%, about 11%, about 12%, about 13%, about 14%, about 15%, about 16%, about 17%, about 18%, and about 19%, including all ranges therebetween, by weight of the Step (b) mixture. In certain embodiments, the concentration of the cryoprotectant is about 1% about 2%, about 3%, about 4%, about 5%, about 6%, about 7%, about 8%, about 9%, about 10%, about 11%, about 12%, about 13%, about 14%, about 15%, about 16%, about 17%, about 18%, about 19% or about 20% by weight of the Step (b) mixture. In particular embodiments, the concentration of the cryoprotectant is about 1% by weight of the Step (b) mixture. In particular embodiments, the concentration of the cryoprotectant is about 3% by weight of the Step (b) mixture. In particular embodiments, the concentration of the cryoprotectant is about 5% by weight of the Step (b) mixture.

### Methods of Cell Transfection

In some embodiments, the present disclosure provides a method of cell transfection comprising contacting one or more target cells with a pharmaceutical composition in accordance with certain embodiments of the present disclosure under conditions suitable to transfect the target cell with a polyplex. In some embodiments, the one or more target cells are eukaryotic cells. In some embodiments, the one or more target cells are one or more of T cells, B cells, blood cells, alveolar cells, pneumocytes, brain neurons, skin neurons, epithelial cells, keratinocytes, iPS cells, fibroblasts, and sweat gland cells.

### Methods of Treatment

In some embodiments, the present disclosure provides a method of treating a disease in a patient in need thereof, comprising administering a therapeutically effective amount of the pharmaceutical composition in accordance with certain embodiments of the present disclosure, such that one or more of the patient's cells are transfected with the polyplex nucleic acid component.

In some embodiments, the present disclosure provides a method of treating a disease in a patient in need thereof, comprising administering a therapeutically effective amount of the pharmaceutical composition in accordance with certain embodiments of the present disclosure, wherein the administration of the composition corrects a defective translation of a target gene in the subject.

In some embodiments, the target gene is selected from the group consisting of COL7A1, LAMB3, ADA, SERPINA1, CFTR, HTT, NF1, PHA, HBS, FERMT1, KRT14, DSP, SPINK5, and FLG. In some embodiments, the gene is COL7A1 and the genetic disease or disorder is a form of epidermolysis bullosa. Epidermolysis bullosa includes Epidermolysis bullosa dystrophica (autosomal recessive), Epidermolysis bullosa dystrophica (localisata variant), Epidermolysis bullosa pruriginosa, Epidermolysis bullosa (pretibial), Epidermolysis bullosa simplex (Dowling-Meara-type), Epidermolysis bullosa simplex (Koebner-type), Epidermolysis bullosa simplex (recessive 1), Epidermolysis bullosa simplex (Weber-Cockayne-type), Epidermolysis bullosa (lethal acantholytic). In some embodiments, the genetic disorder or genetic disease is adenosine deaminase (ADA) deficiency, Alpha-1 Antitrypsin Deficiency, cystic fibrosis, Huntington's Disease, Neurofibromatosis Type 1, Phenylketonuria, Sickle Cell Disease, Sporadic Inclusion Body Myositis, Duchenne muscular dystrophy, Kindler syndrome, Junctional Epidermolysis Bullosa, Dermatopathia pigmentosa reticularis, Naegeli-Franceschetti-Jadassohn syndrome, Netherton Syndrome, Ichthyosis Vulgaris, Atopic Dermatitis, Usher's syndrome, Ehlers-Danlos syndrome, Homozygous Familial Hypercholesterolemia (HoFH), or Crohn's disease.

### EXAMPLES

The following examples are provided to illustrate the present disclosure, and should not be construed as limiting thereof. In these examples, all parts and percentages are by weight, unless otherwise noted. Abbreviations in the examples are noted below.

### Example 1: LBPAE Prepared by Linear Oligomer Combination

Fibroblast gene delivery has yet to show the required efficiency for the therapeutic applications. As described herein, to overcome this limitation, a novel multifunctional LBPAE gene delivery material in accordance with certain embodiments of the present disclosure was prepared via a new linear oligomer combination strategy. The LBPAE in accordance with certain embodiments of the present disclosure achieves superior transfection efficiency and reduced cytotoxicity in difficult-to-transfect fibroblasts HPDF and commonly used 3T3, substantially out-performs the commercially available reagents branched PEI and SuperFect. High LC₅₀ values of LBPAE polyplexes demonstrate their favorable biocompatibility in fibroblast transfections. Mechanism studies indicate that LBPAE equipped with adequate amounts of primary, secondary and tertiary amines is able to condense DNA to nanosized particles with uniform spherical morphology facilitating the cellular uptake and mediating strong buffering capacity to fulfill the efficient endosomal escape. Hydrolysis of the ester bonds on the LBPAE facilitates the DNA release and significantly increases the biocompatibility, allowing for flexible design and adjustment of the polymer/DNA w/w ratios. Along with the high performance of LBPAE in reporter gene deliveries, LBPAE can efficiently deliver the minicircle COL7A1 gene to HPDF and significantly improve the expression of C7, which is critical to maintain the skin integrity. These results demonstrate LBPAE as a high performance non-viral vector in fibroblast-based gene delivery, highlighting its huge potential in genodermatosis treatment and regenerative medicines.

The sequential linear oligomer growth and branching impart the resulting LBPAE more uniform distribution of the linear segments and branching units. Surprisingly, in the difficult-to-transfect HPDF and the commonly used mouse embryo fibroblast (3T3), the newly developed LBPAE exhibits a robust gene transfection ability, the Gluciferase (Gluc) expression much out-performed the commercial gene transfection reagents PEI and SuperFect by up to three orders-of-magnitude and almost 100% green fluorescence protein (GFP) expression was achieved, without inducing obvious cytotoxicity. To decipher the possible mechanisms behind the ultra-potent gene transfection ability of LBPAE in fibroblasts, the multiple extra- and intra-cellular barriers associated with the gene transfection process were investigated. The results illustrate that LBPAE shows a strong DNA binding affinity and can condense DNA to formulate nanosized polyplexes with nearly 100% cellular uptake efficiency. The strong proton buffering capacity along with the biodegradability of LBPAE would also facilitate the LBPAE/DNA polyplex escape from the endo/lysosomes and DNA release in the cytoplasm. Furthermore, LBPAE was used to deliver a minicircle plasmid encoding COL7A1 gene (MCC7) to HPDF and significant upregulation of the C7 expression was detected, showing great promise of LBPAE for the treatment of C7-deficiency genodermatosis such as the devastating and debilitating genetic skin disorder RDEB.

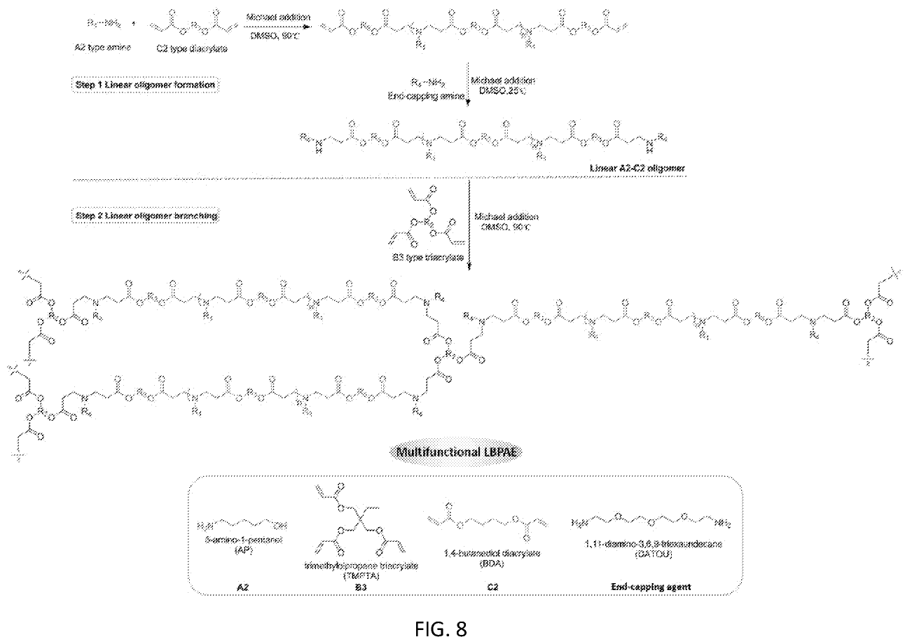

This Example describes a linear oligomer combination strategy to synthesize LBPAE. As illustrated in FIG. 8, this strategy involves two sequential steps: linear oligomer formation and branching. In the first step, A2 type amine reacts with C2 type diacrylate to generate acrylate terminated base oligomer which is further end-capped with a second amine. After purification to remove the unreacted monomers and excess end-capping agent, the linear A2-C2 oligomer is formed. In the second step, B3 type triacrylate is introduced to combine the linear A2-C2 oligomer and yield the LBPAE. The benefits of LBPAEs are two-fold: 1) The length of the linear segments in the obtained LBPAEs would be pre-determined and thus can be tailored easily; 2) The branching units in LBPAEs would be more evenly distributed between the linear segments.

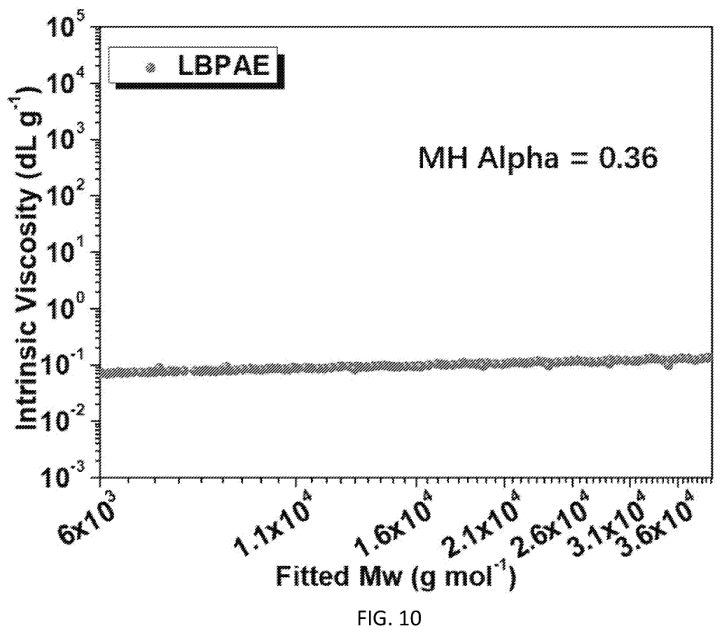

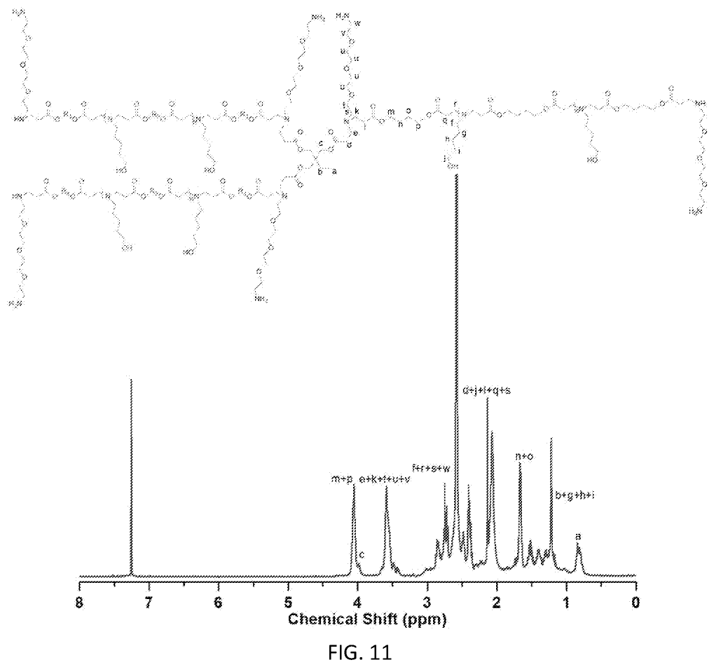

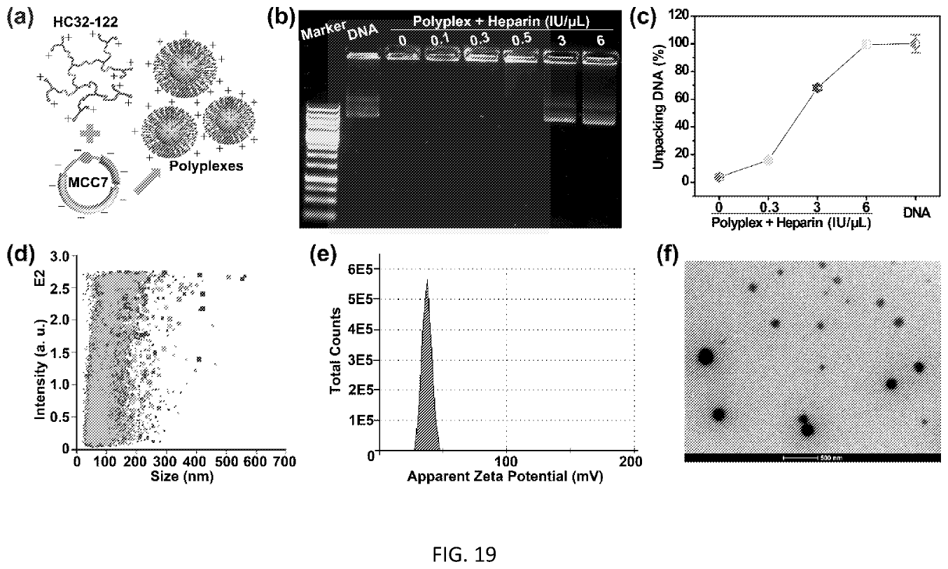

To validate this hypothesis, 5-amino-1-pentanol (AP), trimethylolpropane triacrylate (TMPTA), 1,4-butanediol diacrylate (BDA) and 1,11-diamino-3,6,9-trioxaundecane (DATOU) that have been demonstrated to be effective monomers in the synthesis of PAEs for gene transfection were used as A2, B3, C2 types monomers and end-capping agent for LBPAE synthesis, respectively. AP and BDA with a stoichiometric ratio of 1.2:1 were reacted in dimethyl sulfoxide (DMSO) at 90° C. and the weight average molecular weight (M_{W}) was monitored with gel permeation chromatography (GPC). After 24 hours, when M_{W} of the reaction mixture was approaching 3000 Da, the reaction was stopped by cooling down to room temperature and diluted with DMSO, excess DATOU was then added to end-cap the acrylate terminated base oligomers for 48 hours at 25° C. After removing the unreacted monomers, end-capping agent together with the oligomers of a MW<3000 Da by dialysis in acetone, the linear A2-C2 oligomer with a A4 around 3500 Da and a polydispersity index (PDI) 1.69 was obtained (FIG. 10). To generate LBPAE, the linear A2-C2 oligomer and TMPTA were dissolved in DMSO (the molar ratio of A2-C2: TMPTA was set as 3:1) and reacted at 90° C. When M_{W} was around 10 kDa, the reaction was stopped and excess DATOU was incorporated to consume all the unreacted vinyl groups. And then, the polymer was precipitated in diethyl ether and dried in vacuum oven to give the final LBPAE product. GPC measurement shows that LBPAE has a M_{W} 9.4 kDa with a PDI 2.5 (FIG. 19). The Mark-Houwink (MH) plot alpha value 0.36 validates its highly branched structure (FIG. 10). Chemical composition of LBPAE is confirmed by ¹H NMR (FIG. 11).

### Example 2: LBPAE Achieves Robust Gene Transfection Efficiency and Excellent Cell Viability in Fibroblasts

A viable gene delivery vector can not only achieve high gene transfection efficiency, but also induce minimal cytotoxicity. Nevertheless, in practice, the improvement of transfection efficiency of a gene vector is usually at the cost of its biocompatibility, or vice versa. To evaluate the gene transfection ability of the synthesized LBPAE and identify the most optimal parameters for fibroblast transfection, a series of LBPAE/DNA polyplexes with different w/w ratios were first assessed for the transfection of HPDF and 3T3. Gluc DNA was used as the reporter gene and the gene transfection efficiency was quantified by the Gluc activity measurements after transfection. Alamarblue assays and lethal concentration 50 (LC₅₀) assessments were used to measure the cytotoxicity and the toxicological profile of fibroblasts after transfection. And then, the transfection potency of LBPAE was further validated by flow cytometry using GFP DNA as the reporter gene.

Gluc Expression and Cell Viability of Fibroblasts after Transfection with LBPAE

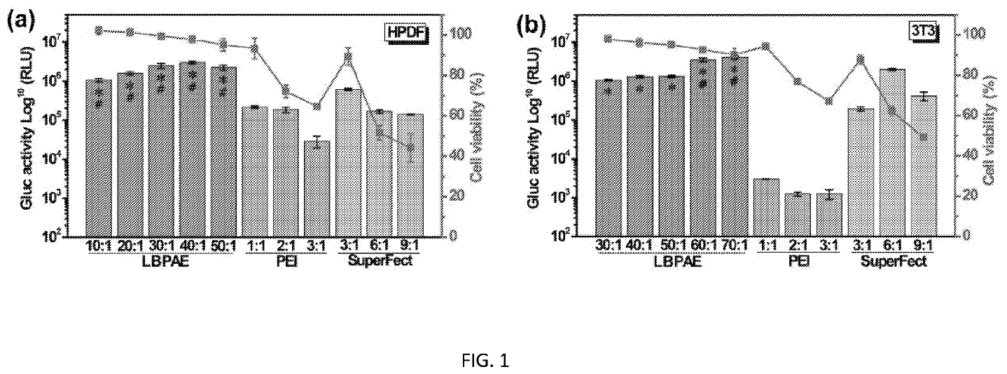

For cationic polymer based gene delivery vector, the polymer/DNA weight ratio (w/w) is a useful parameter for determining both the transfection efficiency and cytotoxicity,^{[5,11]} therefore we first optimized the w/w ratio systematically. Considering that primary cells (e.g., HPDF) are usually fragile to cationic polymers, LBPAE/DNA w/w ratio used for HPDF transfection was increased gradually from 10:1 to 50:1. In order to set up a strong benchmark for comparison, the w/w ratios used for the two dendritic commercial gene transfection reagents PEI and SuperFect were also optimized according to manufacturers' protocols and previous publications.^{[6,13]} FIG. 1a outlined the Gluc activity and cell viability of HPDF after transfection. It is clearly shown that the optimal w/w ratios for PEI and SuperFect gene transfection are 1:1 and 3:1, respectively. Further increase in the w/w ratio not only obviously lowers the Gluc activity, but also substantially increases the cytotoxicity. For example, in comparison with that at the w/w ratio of 3:1, Gluc activity of HPDF after transfection with the SuperFect/DNA polyplexes at the w/w ratio of 9:1 was 3.4-fold lower and the cell viability was decreased from >89% to <44%. In sharp contrast, over the range of the w/w ratios tested, even at the lowest w/w ratio 10:1, Gluc expression of HPDF after transfection by the LBPAE/DNA polyplexes is still stronger than that mediated by the PEI/DNA polyplexes and SuperFect/DNA polyplexes at their optimal w/w ratios. Especially, Gluc activity of HPDF transfected by the LBPAE/DNA polyplexes at the w/w ratio of 40:1 is up to 103-fold higher than that mediated by the PEI/DNA polyplexes. Importantly, LBPAE did not induce obvious cytotoxicity. Even at the highest w/w ratio of 50:1, >95% cell viability was still preserved. In 3T3, the PEI/DNA polyplexes exhibit the similar trend of gene transfection efficiency and cytotoxicity with that in HPDF. Although at the w/w ratio of 6:1 and 9:1, SuperFect/DNA polyplexes show a higher gene transfection efficiency, preserving only 62% and 49% cell viability (FIG. 1b). Again, at all the tested w/w ratios, LBPAE/DNA polyplexes exhibit both strong gene transfection ability and high cell viability. Gluc activity of 3T3 after transfection with the LBPAE/DNA polyplexes is orders-of-magnitude higher than that mediated by the PEI/DNA and SuperFect/DNA polyplexes at their optimal w/w ratios. Surprisingly, at the w/w ratio of 70:1, LBPAE/DNA polyplexes mediated up to 3292-fold higher Gluc activity in comparison with the PEI/DNA polyplexes, while >90% cell viability was still maintained. It should be noted that the same amount of DNA was used between the groups. The much higher w/w ratios employed for the LBPAE/DNA polyplexes mean significantly more LBPAE was used than with either PEI or SuperFect. This further demonstrates the excellent biocompatibility of LBPAE.

Toxicological Profile of the LBPAE

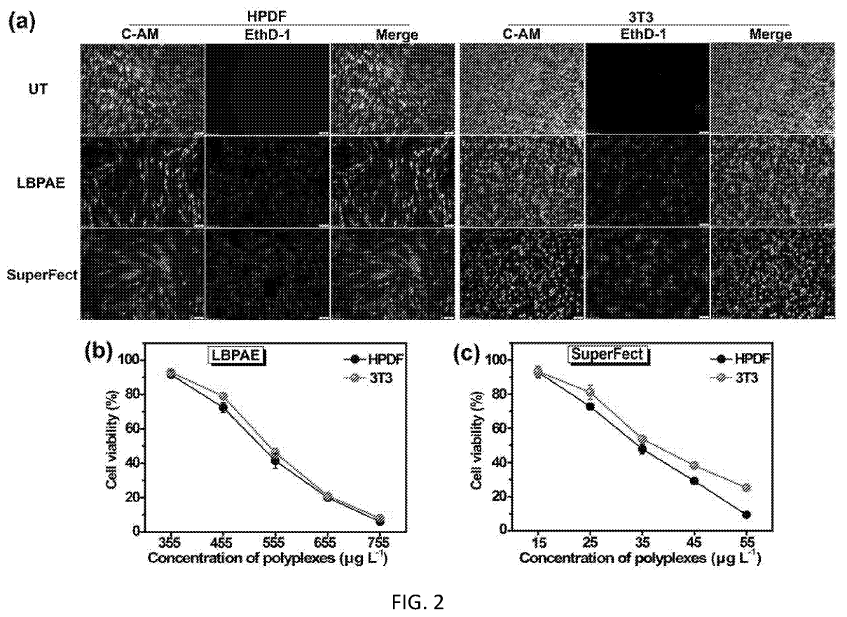

Although >2500 candidates have been developed and screened for gene transfection,^{[28]} so far there is no toxicological study carried out with any of the PAE polymer in fibroblasts. To further validate the biocompatibility of LBPAE in gene transfection, the toxicological profile of LBPAE was determined and the LC₅₀ value was calculated. To this end, LBPAE/DNA polyplexes with the same w/w ratio of 40:1 were used to transfect HPDF and 3T3 with polyplex concentration increased from 355 μg mL⁻¹ to 755 μg mL⁻¹. For comparison, SuperFect/DNA polyplexes were used and the concentration was increased from 15 μg mL⁻¹ to 55 μg mL⁻¹. 24 hours post transfection, cells were simultaneous stained with the green-fluorescent Calcein-AM (C-AM, for live cells) and red-fluorescent ethidium homodimer-1 (EthD-1, for dead cells). The representative fluorescence images of untreated cells and those treated with the LBPAE/DNA polyplexes at the concentration of 555 μg mL⁻¹ and SuperFect/DNA polyplexes at the concentration of 35 μg mL⁻¹ are shown in FIG. 2a. It can be seen that although treated with one order-of-magnitude higher concentration of the LBPAE/DNA polyplexes, HPDF and 3T3 showed similar cell viability with that treated by the SuperFect/DNA polyplexes. Polyplex concentration-dependent cell viability was determined with Alamarblue assay and results are shown in FIG. 2b and FIG. 2c, from which it is calculated that the LC₅₀ values for the SuperFect/DNA polyplexes in HPDF and 3T3 are 35.2 μg mL⁻¹ and 39.5 μg mL⁻¹, respectively. In contrast, the LC₅₀ values of the LBPAE/DNA polyplexes are 538.4 μg mL⁻¹ and 552.3 μg mL⁻¹, corresponding to a 14 and 13-fold increase in comparison with that of the SuperFect/DNA counterparts. SuperFect has been widely used for gene transfection due to its outstanding biocompatibility,^{[29,30]} LBPAE showing a much lower cell-kill effect demonstrates its extremely high biocompatibility, which is of great significance in gene transfection especially for the hard-to-transfect cell types because considerably high polyplex doses or multiple repeat transfections can be used to enhance the transfection efficiency.

GFP Expression Quantified with Flow Cytometry

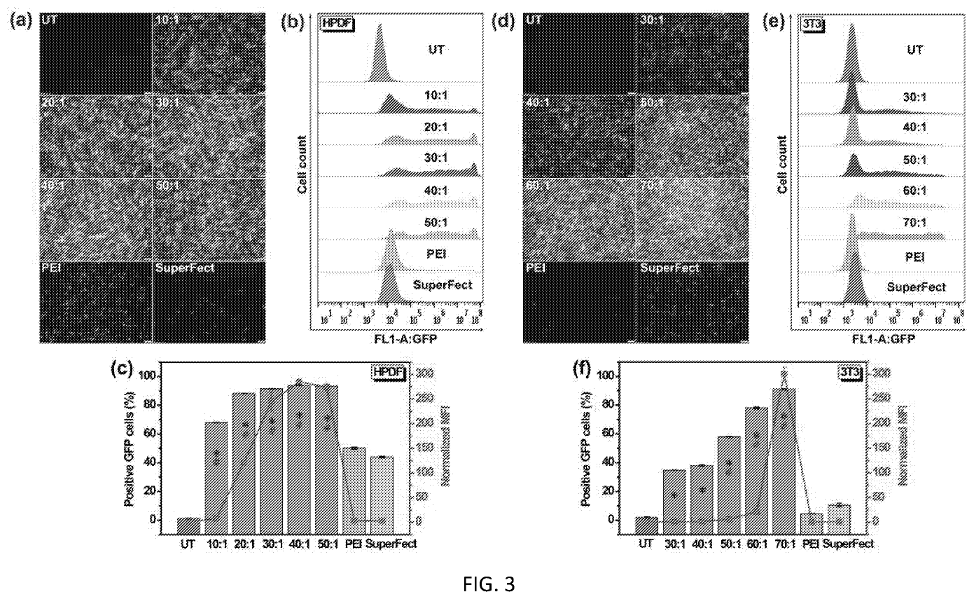

The Gluc DNA was used to quantify the overall transgene expression level mediated by LBPAE, GFP DNA was further used to quantify the percentage of cells transfected. HPDF and 3T3 were transfected with the LBPAE/DNA polyplexes at the same w/w ratios as above. As evidenced by the fluorescence images shown in FIG. 3a, at all the w/w ratios, much more HPDF were transfected by the LBPAE/DNA polyplexes in comparison with that by PEI/DNA and SuperFect/DNA counterparts at their optimal w/w ratios. Flow cytometry measurements show that the percentage of GFP-positive HPDF achieved by the PEI/DNA and SuperFect/DNA polyplexes is only 50% and 44%, respectively. In contrast, LBPAE/DNA polyplexes achieved much higher level of GFP-positive population, as reflected by the far shift of the cell populations responding to the GFP spectrum channel in the histogram distributions (FIG. 3b). The lowest GFP-positive population mediated by the LBPAE/DNA polyplexes is 68% at the w/w ratio of 10:1. When the w/w ratio is above 40:1, >93% of the HPDF are GFP positive. Furthermore, the median fluorescence intensity (MFI) of HPDF transfected by the LBPAE/DNA polyplexes is up to 140-fold higher than that by the PEI/DNA and SuperFect/DNA counterparts (FIG. 3c). In 3T3, the percentage of GFP-positive cells achieved by the LBPAE/DNA polyplexes increased from 35% at the w/w ratio of 30:1 to >91% at the w/w ratio of 70:1, in contrast to 5% and 11% achieved by the PEI/DNA and SuperFect/DNA polyplexes, respectively (FIGS. 3d and 3e). In addition, at the w/w ratio of 70:1, the MFI of 3T3 mediated by the LBPAE/DNA polyplexes was 272 and 230-fold higher compared with that of the PEI/DNA and SuperFect/DNA counterparts (FIG. 3f). These results indicate that LBPAE not only transfects more cell numbers, but also significantly promotes the level of protein expression in the individually transfected cells. Primary fibroblasts are difficult-to-transfect cell types, the fact that LBPAE can mediate >90% gene transfection efficiency in the primary HPDF demonstrates its ultra-potent gene transfection ability. Given that LBPAE has proven to be highly biocompatible over a wide range of w/w ratios with superior gene transfection ability, it can be envisaged that LBPAE will have broad applicability in fibroblast gene transfection.

### Example 3: Possible Mechanisms of LBPAE to Achieve Ultra-Potent Gene Transfection Efficiency and Excellent Biocompatibility

In order to decipher the possible mechanisms behind the high performance of LBPAE in fibroblast transfection, a series of investigations that relate to the multitude extra- and intra-cellular gene delivery barriers including DNA condensation and binding affinity, polyplex size, zeta potential, morphology, proton buffering capacity, degradation rate and DNA release from the polyplexes were conducted.

DNA Condensation and Binding Affinity of LBPAE

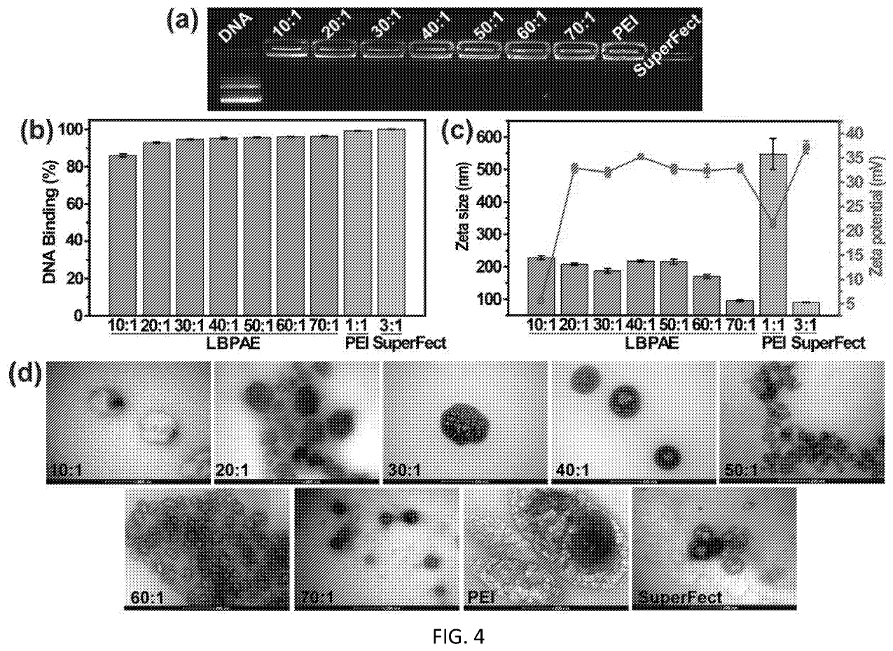

Effective DNA condensation, which can not only protects DNA from degradation by endonuclease but also favors polyplex cellular uptake, is the prerequisite for a successful gene transfection.^{[30]} For cationic polymers, DNA condensation is mainly driven by electrostatic interactions. There are a variety of amines which can partially or fully protonate to generate positive charges. For example, amines which can partially or fully protonate is the multiple terminal primary amines derived from the end-capping agent DATOU, or the multitude backbone tertiary amines derived from the AP. The DNA condensation ability of LBPAE was determined with agarose gel electrophoresis. As shown in FIG. 4a, at all the w/w ratios, no DNA shifting bands were observed, indicating that the negatively charged DNA is shielded by the positively charged LBPAE effectively and thus retained in the agarose wells without migration. Both of the commercial gene transfection reagents PEI and SuperFect show high DNA condensation ability, especially the SuperFect, which condenses the DNA so tight that the DNA staining dye is difficult to gain access to the DNA and thus the DNA band is lighter. The binding affinity between the DNA and LBPAE was further quantified with PicoGreen assay. As shown in FIG. 4b, LBPAE exhibits strong DNA binding affinity at all w/w ratios. In general, the DNA binding affinity increases with the w/w ratio, e.g., from 86% at the w/w ratio of 10:1 to 96% at the w/w ratio of 70:1, demonstrating that more LBPAE leads to stronger electrostatic interaction between the LBPAE and DNA. Comparatively, both PEI and SuperFect show even stronger DNA binding affinity of nearly 100% DNA binding affinity of the LBPAE, PEI and SuperFect correlates very well with their DNA condensation ability. However, it should be noted that a moderate DNA binding affinity is more favorable for gene transfection because over-strong interaction would compromise DNA release from polyplexes.^{[31,32]}

LBPAE/DNA Polyplex Size, Zeta Potential and Morphology

Nanometric size and positive surface charge can facilitate particle cellular uptake through the endocytosis pathway.^{[33,34]} As shown in FIG. 4c, in the physiological solution, over all tested w/w ratios, average sizes of the LBPAE/DNA polyplexes measured with dynamic light scattering (DLS) are all less than 250 nm. In the w/w ratio range from 10:1 to 60:1, polyplexes have the particle sizes between 228 nm and 188 nm. However, when the w/w ratio further increases to 70:1, the polyplex size decreases to 97 nm. Correspondingly, all the polyplexes exhibit a positive zeta potential. At the lowest w/w ratio 10:1, the LBPAE/DNA polyplexes have very low zeta potential of 6 mV. When the w/w ratio is higher than 10:1, the zeta potential significantly increases to >30 mV with the highest 34 mV achieved at the w/w ratio of 40:1. At the same testing conditions, the SuperFect/DNA polyplexes have very small size around 92 nm and high zeta potential around 37 mV. These observations are consistent with the DNA condensation ability and binding affinity of the polymers. In contrast, although of high DNA condensation and binding capacity, the PEI/DNA polyplexes have substantially big size which is >500 nm. Transmission electron microscopy (TEM) was further used to observe the polyplex size and morphology. As shown in FIG. 4d, all the LBPAE/DNA and SuperFect/DNA polyplexes manifest uniform spherical morphologies with the size between 60 nm and 250 nm, similar to that measured by the DLS. Importantly, there is no obvious polyplex aggregation, which demonstrates the high stability of the polyplexes. On the contrary, the PEI/DNA polyplexes exhibit an ellipsoid morphology and the size is much bigger than that of other polyplexes. It is widely accepted that polyplexes with the size <250 nm and moderately positive surface charge are more favorable for cellular uptake while avoiding to induce potential cytotoxicity caused by the excess positive charges.^{[7,9]} The above gene transfection studies have shown that the best w/w ratios for HPDF and 3T3 transfection are 40:1 and 70:1, respectively. This indicates that the most favorable polyplex size and surface charge for effective fibroblast gene transfection may vary substantially according to the cell types. Here, in a broad range of w/w ratios, the LBPAE/DNA polyplexes always have an average size <250 nm and a moderate zeta potential, demonstrating their broad applicability for transfection of diverse cell types to achieve high performance.

### Example 4: Cellular Uptake of LBPAE/DNA Polyplexes

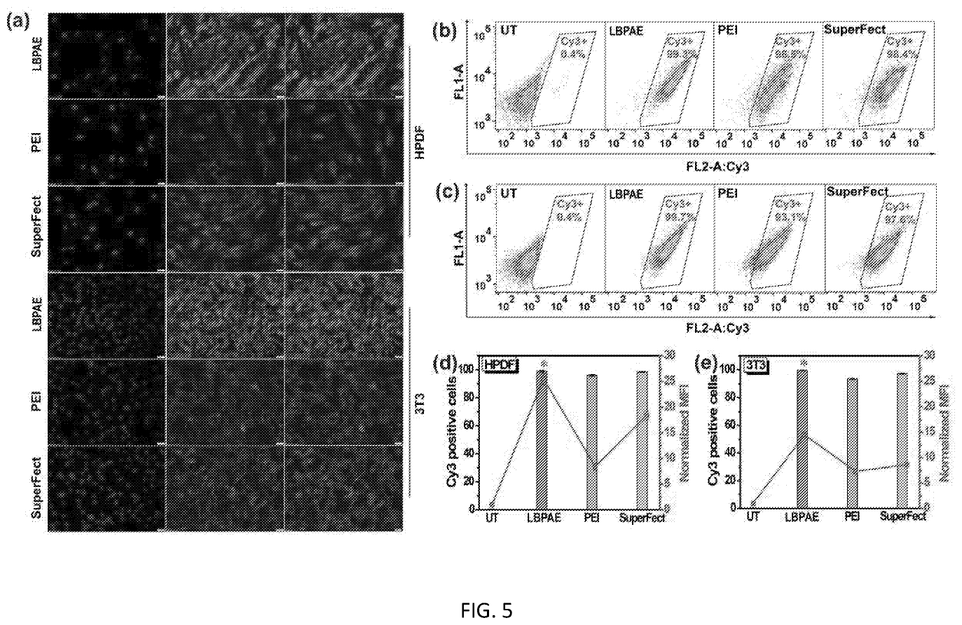

The cellular uptake of polyplexes was further investigated. As shown in FIG. 5a, with the same cell density, 4 hours post transfection, all the polyplexes show high cellular uptake efficiency. Comparatively, much more LBPAE/DNA polyplexes were taken up by the HPDF and 3T3 in comparison with the PEI/DNA and SuperFect/DNA polyplexes, as evidenced by the much stronger red fluorescence observed from the Cy3 labeled DNA. Flow cytometry quantification reveals that the PEI/DNA and SuperFect/DNA polyplexes achieve 96.5% and 98.4% cellular uptake efficiency in the HPDF (FIG. 5b). Even so, the uptake efficiency of the LBPAE/DNA polyplexes is still slightly higher and is almost 100% (99.3%). In the 3T3, similar trend was also observed (FIG. 5c). Furthermore, in the HPDF, the normalized MFI of the LBPAE/DNA polyplexes is 3.05 and 1.39-fold higher than that achieved by the PEI/DNA and SuperFect/DNA counterparts (FIG. 5d). In the 3T3, the out-performance is 1.98 and 1.68-fold, respectively (FIG. 5e). All these results indicate that the PEI/DNA polyplexes have the lowest cellular uptake efficiency while the LBPAE/DNA polyplexes out-performed both the PEI/DNA and SuperFect/DNA counterparts significantly. Collectively, these uptake results correlate with the above polyplex size, zeta potential and gene transfection performance of the different polyplexes very well.

### Example 5: Proton Buffering Capacity Measurement

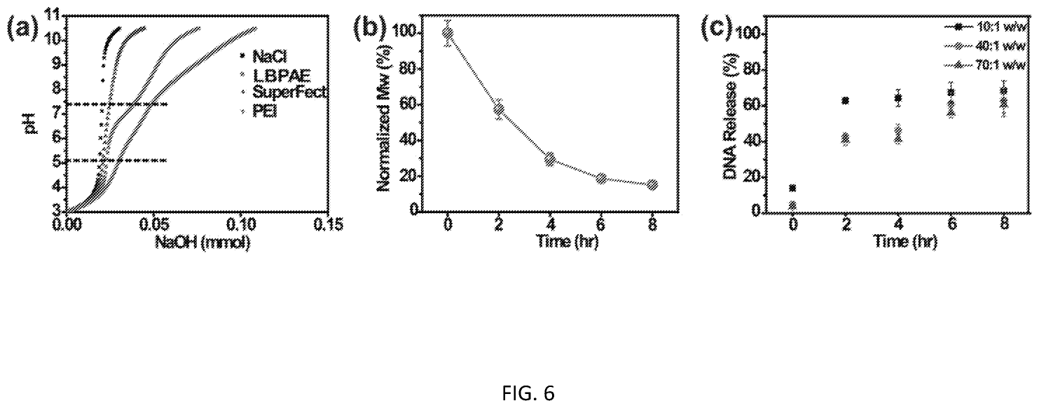

Cationic polymer based polyplexes are usually taken up by cells through the endocytosis pathway, once internalized, they are mainly trapped in the endo/lysosomes. If the polyplexes cannot escape the endo/lysosomal compartments in time, DNA condensed in the polyplexes would be degraded by the digestive enzymes in the acidic compartments. Therefore, endo/lysosomal escape is another major bottleneck to overcome for efficient non-viral gene delivery. The “proton sponge effect” is widely considered the main mechanism for cationic polymers to facilitate polyplexes to escape from endo/lysosomes. Given the mechanism of the “proton sponge effect”, cationic polymers with high content of protonatable secondary and tertiary amines with a pKa close to the endosomal/lysosomal pH are more favorable for polyplex escape from the endo/lysosomes, PEI and SuperFect are the most typical representatives.^{[29]} To verify the proton buffering capacity of LBPAE, acid-base titration was conducted. As shown in FIG. 6a, for the given amount of polymers dissolved in the NaCl solution, it is not surprised that PEI shows the strongest proton buffering capacity with a relatively more flat slope in the acid-base titration curve between the pH 7.4 and 5.1. This is due to the fact that PEI has very high content of primary, secondary and tertiary amines with every third atom a nitrogen in the backbone. After normalization, it is found that the proton buffering capacity of PEI, SuperFect and LBPAE is 5.1 mmol H⁺g⁻¹, 4.6 mmol H⁺g⁻¹ and 1.6 mmol H⁺g⁻¹, respectively (Table 1). Indeed, LBPAE exhibits lower proton buffering capacity than PEI and SuperFect. However, due to the much less cytotoxicity, to effectively promote endo/lysosomal escape of the LBPAE/DNA polyplexes, the w/w ratio can be significantly increased in practical application. For instance, for the HPDF and 3T3 gene transfection, the LBPAE/DNA polyplexes were used at the w/w ratio of 40:1 and 70:1, respectively. Under this condition, the proton buffering capacity of the overall LBPAE used is 12 and 21-fold higher than that of the PEI at the w/w ratio of 1:1, 5 and 8-fold higher than that of the SuperFect at the w/w ratio of 3:1. Based on the “proton sponge effect” hypothesis, the high proton buffering capacity of the LBPAE would cause the increase of the osmotic pressure, leading to swelling and rupture of the endo/lysosomes, and thus release the LBPAE/DNA polyplexes to the cytoplasm in time and efficiently.

TABLE 1
Buffering capacity of LBPAE and commercial reagents.

| Polymer | Buffering capacity per mass (mmol H⁺ g⁻¹) |
|---|---|
| LBPAE | 1.6 |
| PEI | 5.1 |
| SuperFect | 4.6 |

### Example 6: Degradation and DNA Release Assessment of LBPAE

A versatile gene delivery vector can not only effectively condense DNA and protect it from degradation by enzymes, but also can be able to release the condensed DNA from the polyplexes after the nucleus import. For cationic polymers, a series of strategies have been proposed to promote polymer degradation in the cytoplasm and thus facilitate DNA release and reduce accumulative cytotoxicity after gene transfection. However, for efficient gene transfection, a modestly long half-life is required for the gene vectors because too short half-life will lead to insufficient DNA protection and immature DNA release while too long half-life would result in difficulty in polyplex disassociation and DNA release. There are multiple ester bonds on the backbone of PAEs. Under physiological conditions, the ester bonds can be degraded by hydrolysis to yield biocompatible small molecular β-amino acids and diols. It is reported that depending on the chemical composition, LPAEs have a half-life spanning from 1.5 hours to over 6 hours in aqueous environment.^{[7]} For LBPAEs in accordance with certain embodiments and examples of the present disclosure, 43% degradation was observed after 2 hours of incubation at 37° C. The degradation continuously increased to 81% and 85% after 6-hours and 8-hours incubation, respectively (FIG. 6b). The corresponding DNA release from the polyplexes is determined by PicoGreen assay. As shown in FIG. 6c, at the lowest w/w ratio of 10:1, LBPAE/DNA polyplexes have the fastest DNA release rate, after 2 hours of incubation, >60% of the DNA has been released. Comparatively, at the moderate and high w/w ratios of 40:1 and 70:1, the condensed DNA was released from the polyplexes at a slower but similar rate. However, after 6 hours, >60% of the DNA was released. The DNA release profile matches the LBPAE degradation profile demonstrating that LBPAE can release the condensed DNA via hydrolysis spontaneously in physiological conditions without necessitating any additional external triggers. All the results from DNA condensation, binding affinity, polyplex size, zeta potential, cellular uptake, degradation and DNA release correlate with the gene transfection efficiency and biocompatibility of the LBPAE/DNA polyplexes very well, which highlights the manipulation of LBPAE composition, structure and functionality to achieve favorable polyplex properties for high performance gene transfection in fibroblasts.

### Example 7: LBPAE Delivers Functional COL7A1 to Manipulate C7 Expression in HPDF

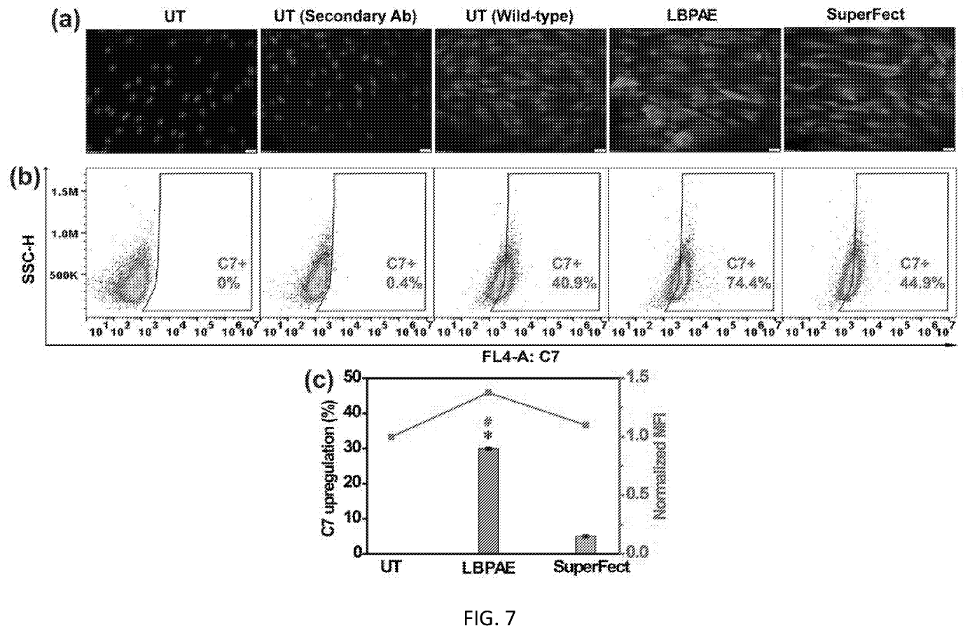

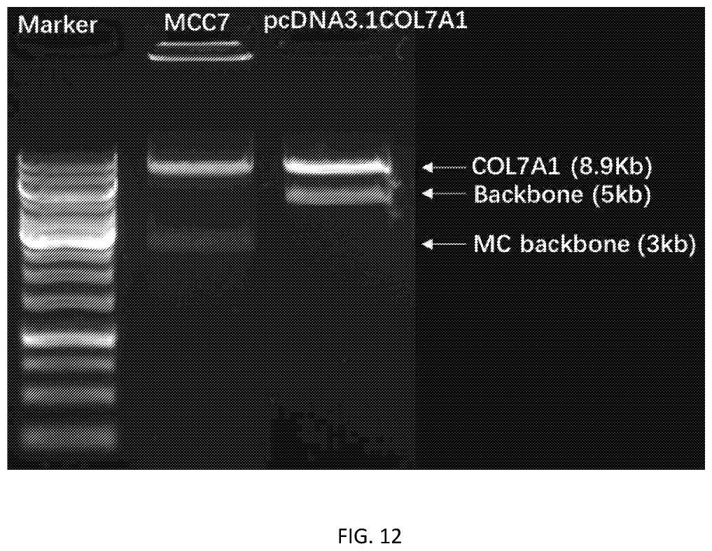

The multifunctional LBPAEs in accordance with certain embodiments and examples of the present disclosure have been demonstrated to be capable of delivering Gluc DNA and GFP DNA to transfect HPDF and 3T3 with ultra-high efficiency and excellent biocompatibility. However, many gene delivery vectors show high level of reporter gene expression, the translation of such success to yield the expression of a functional protein is far more challenging. Hence, the effectiveness of LBPAE was further assessed by delivering a functional COL7A1 gene to promote the expression of C7 in HPDF. Currently, there is no effective cure available beyond palliative care for RDEB. Although both keratinocytes and dermal fibroblasts are capable of producing and secreting C7,^{[35]} the latter are more robust than the former as the target cell types in gene therapy of genodermatosis diseases.^{[21,36]} Minicircle (MC) DNA cassettes have shown a 10-1000 fold higher and more stable non-integrative transgene expression than normal plasmids without the risk of immunogenic responses from the bacterial backbone in standard plasmids.^{[37,38]} Considering that the COL7A1 gene is quite large with about 9 kb cDNA/mRNA transcript, MCC7 encoding the 8.9 kb full-length COL7A1 cDNA with the cytomegalovirus promotor was used to transfect HPDF. As shown in FIG. 12, the MCC7 contains 8.9 kb COL7A1 cDNA and 3 kb backbone, 2 kb less than the pcDNA3.1COL7A1 parental plasmid. It should be noted that the maximum cargo size of retrovirus and adeno-associated virus (AAV) vectors is usually less than 8 kb,^{[39]} both the size of the pcDNA3.1COL7A1 and MCC7 exceeds the gene packaging capacity of the majority viral vectors, therefore efficient COL7A1 gene transfection by LBPAE will have great significance to the gene therapy of RDEB. By combining LBPAE and MC DNA, herein, MCC7 is delivered to manipulate the C7 expression in HPDF with the expectation to enhance its utility in gene therapy of RDEB. FIG. 7a outlined the cyto-immunofluorescence staining images of HPDF four days post transfection with the LBPAE/MCC7 polyplexes. As expected, no obvious C7 expression (red fluorescence) was observed in the untreated group and the group only incubated with the anti-C7 secondary antibody. The wild-type HPDF and the group treated with SuperFect exhibit moderate fluorescence, indicating the production of C7. In contrast, HPDF transfected with the LBPAE/MCC7 polyplexes showed the strongest fluorescence, demonstrating more recombinant C7 expression obtained by LBPAE. Flow cytometry was further used to quantify the C7 expression efficiency. In consist with previous reports,^{[40]} the wild-type of HPDF showed about 41% percent of C7 expression. After transfection with the LBPAE/MCC7 polyplexes, the C7 expression efficiency was significantly increased to 74.4%, in comparison with 44.9% achieved by the SuperFect/MCC7 polyplexes (FIG. 7b). Moreover, 40% enhancement in MFI was also realized by the LBPAE/MCC7 polyplexes, in contrast to 10% achieved by the SuperFect/MCC7 counterparts (FIG. 7c). All these results demonstrate that LBPAE can not only effectively deliver MCC7 to increase the overall population of C7-expressing HPDF, but also enhance the C7 level in individual HPDF. In addition, our preliminary study showed that in the C7 null RDEB fibroblasts (RDEBF), after transfection with the LBPAE/MCC7, the C7 expression efficiency was restored to around 40%, further optimization of the transfection and quantification of the C7 expression in RDEBF are still undergoing. These findings indicate that LBPAE has strong payload capacity to deliver the large cDNA in skin primary cells. The primary dermal fibroblasts can be further engineered by this polymeric vector to secret potent cellular C7 which is pivotal to strengthen the dermal-epidermal junction. Although the non-viral gene therapy requires repeated applications, for the genetic C7 dysfunction skin diseases, given the apparent wound sites and the good medication accessibility that multiple topical administrations are much safer than systemic gene deliveries, therefore, LBPAE holds great promise for fibroblast-based gene therapies to restore or enhance the C7 expression and thus reverse the disease phenotype of RDEB.

### Example 8: Experimental

Materials: Trimethylolpropane triacrylate (TMPTA), 5-amino-1-pentanol (AP), 1,11-diamino-3,6,9-trioxaundecane (DATOU), sodium chloride (NaCl), sodium hydroxide (NaOH), branched polyethylenimine (PEI, M_{w}=25 kDa), lithium bromide (LiBr), dimethyl sulfoxide (DMSO), diethyl ether, deuterated chloroform (CDCl3), hydrochloric acid solution (HCl), Hank's balanced salt solution (HBSS), tris acetate-EDTA buffer (TAE), trypsin EDTA solution (0.25%), Dulbecco's Modified Eagle Medium (DMEM), penicillin-streptomycin (P/S), agarose, paraformaldehyde (PFA), 0.1% Triton X-100, monoclonal Anti-Collagen VII antibody produced in mouse and goat serum were purchased from Sigma-Aldrich. Sodium acetate (3.0 M, Sigma-Aldrich) was diluted to 0.025 M prior to use. 1,4-butanediol diacrylate (BDA) was purchased from VWR and used as received. Dimethylformamide (DMF) was purchased from Fisher Scientific. Fetal bovine serum (FBS) purchased from Gibco was filtered through a 0.2 μm filter before use. HPDF and 3T3 cells were purchased from Lonza and ATCC, respectively. For culture and subculture of the HPDF, fibroblast basal medium, FGM-2 SingleQuots, Clonetics Reagent Pack including HEPES buffered saline solution, trypsin EDTA solution (0.25%), and trypsin neutralizing solution were purchased from Lonza. Cell secreted Gaussia princeps luciferase plasmid (GLuc DNA) and BioLux™ Gaussia luciferase assay kit were purchased from New England Biolabs UK. Expansion and purification of the Gluc DNA were performed using the Giga-Prep kit (Qiagen) as per protocols. Green fluorescent protein plasmid (GFP DNA) was purchased from Aldevron. pcDNA3.1COL7A1 plasmid was kindly provided by Dr. Andrew South at University of Dundee (UK). MCC7 was constructed by inserting the COL7A1 sequence originated from the pcDNA3.1COL7A1 to the MN511A-1 cassette offered from System Biosciences, production of minicircle DNA was according to the user manual of System Bioscience. SuperFect gene transfection reagent was purchased from Qiagen. LIVE/DEAD Viability/Cytotoxicity kit, goat anti-mouse IgG (H+L) highly cross-adsorbed secondary antibody, Alexa Fluor 568 and Alexa Fluor 647 were purchased from Thermo Fisher Scientific. Alamarblue assay kit, SYBR safe DNA gel stain, IC fixation buffer and 10× Bioscience Permeabilization buffer were purchased from Invitrogen. Cy3 DNA labelling kit was purchased from Minis and used as per protocols. 4′,6-diamidino-2-phenylindole (DAPI) and PicoGreen assay kit were purchased from Life Technologies and used as per manufacturers' protocols. 1× Dulbecco's phosphate buffered saline (PBS) was purchased from Life Technologies. Mounting medium with DAPI was purchased from Abcam.

Synthesis and characterization of LBPAE: LBPAE was synthesized through a linear oligomer combination strategy. Firstly, BDA and AP with a stoichiometric ratio of 1.2:1 was dissolved in DMSO at 100 mg mL and then reacted at 90° C. An Agilent 1260 Infinite gel permeation chromatography (GPC) equipped with a triple detector ((a refractive index detector (RI), a viscometer detector (VS DP) and a dual light scattering detector (LS 15° and LS 90°)) was used to monitor the growth of the weight average molecular weight (M_{w}), number average molecular weight (Mₑ), and polydispersity index (PDI). For GPC measurement, 20 μL of the reaction mixture was taken and diluted in 1 mL DMF, and then filtered through a 0.45 μm filter. DMF with 0.1% LiBr was utilized to elute the GPC columns (Polar Gel-M, 7.5×300 mm, two in series) at a flow rate of 1 mL min⁻¹ at 60° C. Linear poly(methyl methacrylate) (PMMA) standards were used for the calibration of the GPC columns. When the M_{W} was approaching 3000 Da, the reaction was stopped by cooling down to room temperature and diluted with DMSO, and then excess end-capping agent DATOU was added, the reaction was continued for another 48 hours to yield the DATOU end-capped linear A2-C2 oligomer, which was purified by dialysis with acetone for three days and then dried in a vacuum oven to remove the solvent. Next, the linear A2-C2 oligomer was dissolved in DMSO and reacted with the branching monomer TMPTA at 90° C. When the M_{w} was around 10 kDa, the reaction mixture was cooled down to room temperature and excessive DATOU was added to consume all the unreacted vinyl groups for another 48 hours. The polymer was then purified by precipitation with diethyl ether three times, freeze dried for two days, and stored at −20° C. for further studies. To measure the molecular weight (M_{w}), PDI and Mark-Houwink (MH) plot alpha value (a) of the final product, 10 mg LBPAE was dissolved in 2 mL DMF and GPC measurement was carried out as mentioned above. Chemical composition and purity of LBPAE were determined with H NMR on a 400 MHz Varian Inova spectrometer. The sample was reported in parts per million (ppm) relative to the solvent CDCl3 (7.24 ppm) or internal control (tetramethylsilane 0.00 ppm).

Determination of the Mark-Houwink alpha parameter: The determination of the Mark-Houwink alpha parameters of the polymers was conducted on a 1260 Infinite GPC system with a refractive index detector (RI), a viscometer detector (VS DP) and a dual angle light scattering detector (LS 15° and LS 90°). To prepare polymers for analysis, 10.0 mg samples were dissolved in 2 mL DMF and then filtered through a 0.45 μm filter. GPC columns (30 cm PLgel Mixed-C, two in series) were eluted with DMF and 0.1% LiBr at a flow rate of 1 mL/min at 60° C. Columns were calibrated with linear poly(methyl methacrylate) standards (PMMA). The GPC data were analyzed using universal calibration.

LBPAE/DNA polyplex preparation: For polyplex preparation, LBPAE was first dissolved in DMSO to 100 mg mL⁻¹ stock solution. According to the LBPAE/DNA weight ratio (w/w), the required amount of LBPAE stock solution and DNA solution were diluted with sodium acetate buffer (0.025 M, pH=5.2) to equal volume, respectively. And then, the LBPAE solution was added to the DNA solution, mixed by vortex for 10 seconds and kept undisturbed for 10 minutes at room temperature to allow for polyplex formation.

DNA condensation by LBPAE: Agarose gel electrophoresis was used to determine the DNA condensation ability of LBPAE. 1 μg DNA was used for each sample preparation, polyplexes with a series of w/w ratios were prepared as above. After that, 20 μL of the polyplex solution was loaded into the wells in the agarose gel (1% in 1×TAE buffer) containing 10 μL SYBR safe DNA gel stain, naked DNA was used as the control. Gel electrophoresis was performed in 1×TAE buffer at 120 V for 40 minutes and the images were captured using a Syngene's G:BOX.

DNA binding affinity of LBPAE: PicoGreen assay was used to quantify the DNA binding affinity of LBPAE. 0.25 μg DNA was used for each sample preparation. Polyplexes with different w/w ratios were prepared in 15 μL sodium acetate buffer and then mixed with 15 μL PicoGreen working solution and incubated for five minutes. Afterwards, 220 μL of 1×PBS buffer was added to dilute the polyplexes in a black 96-well plate. Fluorescence intensity (F) of the polyplex solution was measured by a SpectraMax M3 plate reader with the excitation at 490 nm and emission at 535 nm in quadruplicate. DNA binding affinity of LBPAE was defined by the following equation:

$$DNA binding affinity ( \% ) = ( 1 - \frac{{F}_{s a m p l e} - {F}_{B l a n k}}{{F}_{D N A} - {F}_{B t a n k}} ) \times 100 \qquad ( 1 )$$

Proton buffering capacity of LBPAE: Proton buffering capacity of LBPAE was determined by acid-base titration. 0.1 M NaCl solution was used as the background control, PEI and SuperFect were used as the positive controls. A Mettler Toledo S20 pH meter was used to measure the pH values. 10 mg LBPAE, 5 mg PEI or 0.8 mg SuperFect was dissolved in 20 mL 0.1 M NaCl solution. pH values of the solution were adjusted to 3.0 with 1.0 M HCl solution and then titrated to 10.5 using 0.1 M NaOH solution. The proton buffering capacity of LBPAE (mmol g⁻¹) was calculated using the following equation:

$$Proton buffering capacity ( mmol {g}^{- 1} ) = \frac{1.0 x {V}_{HCl buffered from pH 5.1 - 7.4}}{Polymer mass} \qquad ( 2 )$$

Degradation profile of LBPAE: To measure the degradation profile, LBPAE was dissolved in PBS at a concentration of 10 mg mL and kept shaking at 180 rpm under 37° C. At the time points of 0, 2, 4, 6 and 8 hours, 1 mL of the solution was taken out and frozen immediately. After freeze drying, the sample was dissolved in 1 mL DMF. M_{W} of the sample was measured by GPC as mentioned before in triplicate. The percentage of LBPAE degradation was defined as following:

$$DNA degradation rate ( \% ) = ( 1 - \frac{{M}_{w . cur r e n t}}{{M}_{w . original}} ) \times 100 \qquad ( 3 )$$

Polyplex size and zeta potential determination: Polyplex sizes and zeta potentials were measured using a Malvern Instruments Zetasizer (Nano-Z590, scattering angle 173°, 633 nm laser). 4 μg DNA was used for each sample preparation, polyplexes with a series of w/w ratios were prepared as mentioned above and diluted with 800 μL deionized water, and then transferred to Zetasizer cells or cuvettes. Size and zeta potential measurements were carried out at 25° C. in quadruplicate.

Polyplex morphology characterization by transmission electron microscopy: (TEM): Morphologies of the LBPAE/DNA, PEI/DNA and SuperFect/DNA polyplexes were characterized by TEM. 80 μL polyplex solution containing 2 μg DNA was prepared as before, washed with deionized water twice to remove the salts and then re-suspended in 10 μL deionized water. 2.5 μL of the re-suspended polyplex solution was cast onto a Formvar support film on 200 mesh copper grids and freeze-dried immediately. The TEM images were captured on a FEI Tecnai 120 TEM at 120 kV at UCD Conway Imaging Core Centre.

DNA release from polyplexes: DNA release from the polyplexes can be determined by measuring the reduction of binding affinity by LBPAE using PicoGreen assay. LBPAE/DNA polyplexes with the w/w ratios of 10:1, 40:1 and 70:1 were prepared as above and kept shaking at 180 rpm and 37° C. At the time points of 0, 2, 4, 6 and 8 hours, 100 μL of the polyplex solution was taken out and the DNA binding affinity was measured immediately as before in quadruplicate. The DNA release rate from the polyplexes was determined as following:

$$DNA release rate ( \% ) = \frac{{F}_{Sample} - {F}_{Blank}}{{F}_{D N A} - {F}_{Blank}} \times 100 \qquad ( 4 )$$

Cell culture: HPDF were cultured in fibroblast basal medium and supplemented with the FGM-2 SingleQuots contains 2% of FBS. 3T3 were cultured in DMEM containing 10% FBS and 1% penicillin/streptomycin (P/S). Both types of cells were cultured at 37° C., 5% CO₂ in a humid incubator under standard cell culture conditions.

Gene transfection ability of LBPAE quantified by Gluc expression: Gene transfection ability of LBPAE in HPDF and 3T3 was first evaluated with Gluc expression using Gluc DNA as the reporter gene. Cells were seeded in 96-well plates at a density of 1×10⁴ cells per well for 3T3 and 2×10⁴ cells per well for HPDF in 100 μL medium and incubated for one day prior to transfection. The commercial gene transfection reagents PEI and SuperFect were optimized as per manufactures' protocols. To this end, 0.5 μg Gluc DNA was used for each well, the w/w ratio for the PEI/DNA polyplexes was varied from 1:1, 2:1 to 3:1, the w/w ratio for the SuperFect/DNA polyplexes was varied from 3:1, 6:1 to 9:1. For LBPAE gene transfection, the same amount of 0.5 μg Gluc DNA was used for each well, LBPAE/DNA polyplexes with different w/w ratios were prepared in 20 μL of sodium acetate buffer as mentioned above and then diluted with 80 μL cell culture medium. Cell culture medium in the 96-well plates was removed and 100 μL polyplexes-containing medium was added. 4 hours later, the polyplexes-containing medium in the plates was replaced with 100 μL fresh medium, the cells were incubated for another 44 hours. Gluc activity of the cells after transfection was measured with Gluc assay as per standard protocols in quadruplicate. Briefly, 20 μL of the supernatant in the 96-well plates was taken out and 50 μL of the Gluc assay solution was added. The luminescence intensity was measured using a SpectraMax M3 plate reader and the Gluc activity was directly plotted in terms of relative light units (RLU).

Gene transfection ability of LBPAE quantified by GFP expression: Gene transfection ability of LBPAE was further evaluated with GFP expression. HPDF and 3T3 cells were seeded in 24-well plates at a density of 5×10⁴ cells per well in 500 μL media and incubated for one day prior to transfection. 2 μg GFP DNA was used for each well, polyplexes with different w/w ratios were prepared in 100 μL sodium acetate buffer and then mixed with 400 μL cell culture medium. The medium in the cells was removed and the polyplexes-containing medium was added. 4 hours later, the polyplexes-containing medium was replaced with 500 μL fresh medium and the cells were incubated for another 44 hours. After that, the cells were washed with HBSS and imaged with a fluorescence microscope (Olympus IX81). To quantify the GFP expression with flow cytometry, the transfected cells were digested with trypsin EDTA and washed with HBSS twice, and then re-suspended in PBS with 2% FBS. The flow cytometry measurements were carried out on an Accuri C6 system in triplicate, at least 10,000 cells were counted for each sample. The median fluorescence intensity (MFI) of cells was quantified with Flowjo software. Cells transfected with PEI and SuperFect were used as the positive controls, untreated cells were used as the negative control.

Cell viability measured with Alamarblue assay: Viability of the HPDF and 3T3 cells after transfection was measured with Alamarblue assay. To this end, 48 hours post transfection, the cell supernatants were removed and the cells were washed with HBSS twice. And then 10% of Almarblue solution in HBSS was added and the cells were incubated for another 1˜3 hours. Afterwards, the Alamarblue solution in wells was transferred to a flat-bottomed 96 well-plate and the fluorescence intensity was measured at 590 nm using a SpectraMax M3 plate reader in quadruplicate. Cells without any polyplex treatment were used as positive control, the fluorescence intensity was normalized as 100% cell viability.

Toxicological profile of LBPAE: Toxicological profile of LBPAE was determined by lethal concentration 50 (LC₅₀) assessments. LIVE/DEAD Viability/Cytotoxicity kit was used to stain the live and dead cells. Cells were seeded in 96-well plates at a density of 2×10⁴ per well for HPDF and 1×10⁴ per well for 3T3 in 100 μL medium. The next day, LBPAE/DNA polyplexes and SuperFect/DNA polyplexes were prepared, five different doses of the polyplexes were used to transfect the cells as mentioned above. 24 hours post transfection, the cell culture medium was removed and replaced with HBSS containing calcein-AM (C-AM) (1:5000) and ethidium homodimer-1 (EthD-1) (1:500), the cells were incubated for another 20 minutes. And then, the cells were washed with HBSS and imaged with a fluorescence microscopy (Olympus IX81). The cell viability quantified with Alamarblue assay in quadruplicate was used for the LC₅₀ calculation.

Polyplex cellular uptake: For polyplex cellular uptake studies, Gluc DNA was labelled with a Cy3 (a red fluorescent dye) labelling kit as per the recommended protocol. Fibroblasts were seeded in a 24-well plate at a density of 5×10⁴ cells per well. 0.5 μg labelled DNA was used for each well, gene transfection was carried out the next day as mentioned before. 4 hours post transfection, the cells were washed with HBSS, fixed with 4% PFA, permeabilized with 0.1% triton-100, stained with DAPI and then imaged with a fluorescence microscope (Olympus IX81). To quantify the cellular uptake efficiency with flow cytometry, after transfection, the cells were digested with trypsin EDTA and washed with HBSS twice, and then re-suspended in PBS with 2% FBS, the percentage of Cy3 positive cells and the MFI were quantified on an Accuri C6 flow cytometry system in triplicate.

Detection of C7 expression in HPDF by cyto-immunofluorescence staining: To detect C7 expression with cyto-immunofluorescence staining, HPDF were seeded in 8-well chambers (Ibidi) at a density of 1.5×10⁴ cells per well. The next day, 1 μg MCC7 was used for each well, gene transfection was carried out as mentioned above using LBPAE/DNA polyplexes at the w/w ratio of 40:1 and SuperFect/DNA polyplexes at the w/w ratio of 3:1. 48 hours post transfection, the cells were fixed with 4% PFA, permeabilized with 0.1% triton-100, blocked with 5% goat serum in 1×PBS for 1 hour at room temperature, and then incubated with primary antibody of monoclonal anti-collagen and Type VII antibody produced in mouse in blocking buffer (antibody dilution: 1:200) at 4° C. overnight. The next day, the cells were incubated with Alexa-568 goat anti-mouse IgG (H+L) highly cross-adsorbed secondary antibody at a 1:800 dilution for 1 hour in dark and DAPI at room temperature. The immunofluorescence images were taken using a fluorescence microscope (Olympus IX81). Cells without antibody treatment and treated only with secondary antibody were used as control groups.

C7 expression in HPDF quantified by flow cytometry: To quantify the C7 expression with flow cytometry, HPDF were seeded in a 24-well plate at a density of 5×10⁴ cells per well and transfected 24 hours later. 2 μg MCC7 was used for each well and the transfection was carried as above using LBPAE/DNA polyplexes at the w/w ratio of 40:1 and SuperFect/DNA polyplexes at the w/w ratio of 3:1. Four days post transfection, the cells were digested by trypsin EDTA, fixed with the IC fixation buffer (2% PFA), permeabilized with the permeabilization buffer (1×PBS/1% BSA/0.1% Saponin) and blocked in goat serum (10% in permeation solution), and then incubated with the primary antibody of monoclonal anti-collagen and Type VII antibody produced in mouse in the blocking buffer at a 1:50 dilution for 1 h at room temperature. Afterwards, the cells were further incubated with the Alexa-647 goat anti-mouse IgG (H+L) cross-adsorbed secondary antibody at a dilution of 1:3000 in the permeation buffer in dark. Finally, the cells were re-suspended in PBS and analyzed by flow cytometry in triplicate. Cells without antibody treatment and treated only with secondary antibody were used as control groups.

Statistics: SPSS Statistics for windows version 24 (IBM Corp., Armonk, N.Y., USA) was used for statistics analyses. Student's t-test was used to analyze all the gene transfection data, data was shown as mean±standard deviation. LC₅₀ values were calculated by linear regression analysis. P value <0.05 was considered statistically significant.

### Example 9: HPAE Polyplexes Comprising COL7A1 for Gene Delivery to Recessive Dystrophic Epidermolysis Bullosa Keratinocytes

The following example describes the transfection results for polyplexes comprising minicircle COL7A1 and a four highly branched poly(β-amino ester)s (HPAEs) of the present disclosure having molecular weights (M_{w}) of about 10 kDa, 20 kDa, 30 kDa and 40 kDa and using HPAE:COL7A1 DNA weight ratios of 10:1, 30:1 and 50:1.

HPAE synthesis and characterization: The HPAEs were prepared in two stages. In stage one, the monomers 5-amino-1-pentanol, trimethylolpropane triacrylate, 1,4-butanediol diacrylate were reacted to provide a highly branched C32 (Poly(5-amino-1-pentanol-co-1,4-butanediol diacrylate)) (“HC32”). In stage two, the HC32 was reacted with 1,11-diamino-3,6,9-trioxaundecane (DATOU) to provide HPAE (“HC32-DATOU”).

Four HC32-DATOU polymers with different molecular weight (MW) were synthesized via the “A2+B3+C2” Michael addition strategy. HC32 base polymers were first synthesized. Briefly, the A2 monomer AP (9.0 mmol, 0.923 g), B3 monomer TMPTA (0.5 mmol, 0.148 g) and C2 monomer C (10.0 mmol, 1.98 g) were dissolved in 3.1 mL DMSO, and then reacted at 90° C. Gel permeation chromatography (GPC) was used to monitor the growth of M_{W} and polydispersity index (PDI). 20 μL of reaction sample was taken at different time points, followed by diluting in 1 mL DMF and filtering through a 0.2 μm filter prior to GPC measurement on an Agilent 1260 Infinite GPC equipped with a triple detector: a refractive index detector (RI), viscometer detector (VS DP) and dual light scattering detector (LS 15° and LS 90°). DMF and 0.1% LiBr was used to elute the GPC column (PolarGel-M, 7.5×300 mm, two in series) at a flow rate of 1 mL/min at 60° C. GPC columns were calibrated with the linear poly(methyl methacrylate) (PMMA) standards.

When the weight average molecular weight (M_{w}) of the based polymer was approaching target values (around 10, 20, 30 and 40 kDa, respectively), the reaction was stopped by diluting the reaction solution in DMSO to 100 mg/mL. Afterwards, the end-capping agent DATOU (10.0 mmol, 1.92 g) dissolved in DMSO (100 mg/mL) was used to end-cap the HC32 base polymers through Michael addition at room temperature (RT) for 48 h to obtain the HC32-DATOU polymers, which were purified by precipitation with diethyl twice to remove the excess monomers, oligomers and end-capping agent.

The final HC32-DATOU products were dried in a vacuum oven for 24 h and then freeze-dried for another 24 h to remove the residual solvents. To measure the M_{W} and PDI of the final products, 10 mg sample was dissolved in 1 mL DMF and GPC measurements were carried out as mentioned above. Proton Nuclear Magnetic Resonance (¹H NMR) was utilized to confirm chemical compositions and purity of the HC32-DATOU polymers, which were dissolved in CDCl₃ and ¹H NMR spectra was acquired on a 400 MHz Varian Inova spectrometer. Sample was reported in parts per million (ppm) relative to the solvent (7.24 ppm) or internal control (tetramethylsilane 0.00 ppm).

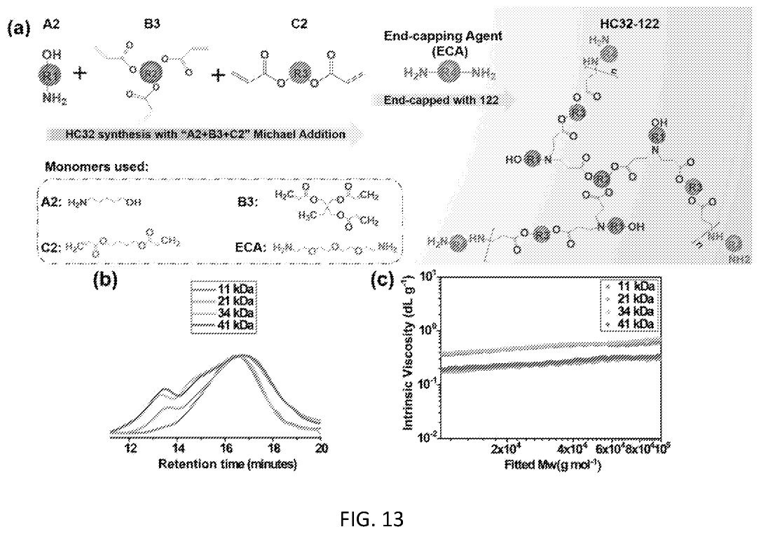

FIG. 13 shows that by increasing the polymerization time of the base polymers, four HC32-DATOU polymers with different M_{W}s were obtained. M_{W} of HC32-DATOU increased from 11 kDa to 41 kDa without gelation, demonstrating high flexibility of the “A2+B3+C2” Michael addition strategy in controlling the HAPE M_{W}. MH plot alpha values of all HC32-DATOU polymers are below 0.5 (FIG. 13c), indicating their highly branched structures. The following table shows PDI and MH plot alpha values for the HC32-DATOU polymers

| Mw (kDa) | PDI | alpha values |
|---|---|---|
| 11 | 3.4 | 0.44 |
| 21 | 6.3 | 0.38 |
| 34 | 8.5 | 0.29 |
| 41 | 12.9 | 0.33 |

MCC7 Biosynthesis.

Regular plasmid pcDNA3.1COL7A1 was obtained. MCC7 was biosynthesized by inserting the COL7A1 sequence originated from the pcDNA3.1COL7A1 to the MN511A-1 cassette offered from System Biosciences with cytomegalovirus promoter, induction and production of minicircle DNA were carried out according to the user's manual of System Bioscience and the published phiC31 plus 1-Scel digest system of minicircle technology (Gaspar, V.; de Melo-Diogo, D.; Costa, E.; Moreira, A.; Queiroz, J.; Pichon, C.; Correia, I.; Sousa, F. Minicircle DNA Vectors for Gene Therapy: Advances and Applications. Expert Opin. Biol. Ther. 2015, 15 (3), 353-379. https://doi.org/10.1517/14712598.2015.996544.). To confirm the biosynthesis of MCC7, DNA digestion study was carried out. To this end, 0.5 pcDNA3.1COL7A1, parental plasmid (MN511A-1-COL7A1) and MCC7 were digested by 1 μL EcoRI and then subjected to agarose gel electrophoresis at 100 V for 40 minutes. Then images were visualized using a Syngene's G:BOX.

Polyplex Preparation and Formulation

HC32-DATOU was dissolved in DMSO to a 100 μg/μL stock solution which was stored at −20° C. for the following studies. DNA was dissolved in TE buffer and stored at −20° C. as well. SA buffer was diluted to 0.025 M prior to use.

For standard polyplex preparation, according to the polymer/DNA weight ratio (w/w), DNA and polymer were dissolved in the SA buffer to equal volume, respectively. The polymer solution was added to the DNA solution, mixed for 10 seconds using a vortex and incubated for another 10 minutes at RT to allow the polyplex formation. For the formulation study, typically, 5 μg GFP plasmid DNA and 150m HC32-DATOU were dissolved in 200 μL SA, respectively, to formulate the HC32-DATOU/DNA polyplexes (w/w=30:1). The polyplexes were either immediately used as the fresh ones, or stored at RT, 4° C., −20° C. and −80° C., or lyophilized prior to transfection.

For lyophilization, sucrose was added to the polyplex solution to final sucrose concentrations of 0%, 1%, 3% and 5%, respectively. All samples were frozen at −80° C. for 1 h and then immediately subjected freeze dry with a Christ Alpha 1-2 LDplus Freeze Dryer at −55° C. for 24 h. Afterwards, the polyplexes were reconstituted with the original volume of SA and used for transfection.

After optimization of the polyplex formulation procedure (see Example 10, below), HC32-DATOU complexed with Gluc-encoding DNA stored at different conditions was used to evaluate the feasibility for long-term storage of polyplexes prior to transfection applications. Here, 0.5 μg DNA at 30:1 polymer/DNA w/w ratio was used for each well in 96-well plates.

DNA Condensation and Heparin Release Studies

To assess the DNA condensation ability of HC32-DATOU and the physical stability of the HC32-DATOU/DNA polyplexes, DNA condensation assay and heparin release assay were performed using agarose gel electrophoresis. 0.5 μg DNA (MCC7) was used for each sample and polyplexes were prepared at the w/w ratio of 30:1. Aqueous heparin solution was added in the polyplex solution with concentration increasing from 0.1-6 IU/μL. Naked DNA and HC32-DATOU/MCC7 polyplexes without heparin were used as the controls. All samples were incubated at RT for 2 h and then loaded on a 1% agarose gel stained with 10 μL SYBR safe DNA stain. Electrophoresis was performed in 1×TAE buffer at 100 V for 1 h.

PicoGreen Assay

PicoGreen assay was used to quantify the DNA binding affinity of HC32-DATOU and DNA release in the presence of heparin. HC32-DATOU/MCC7 polyplexes were prepared with 0.2 μg DNA at the 30:1 w/w ratio, and then heparin was introduced to the polyplex solution at the concentration of 0.3 IU/μL, 3 IU/μL and 6 IU/μL, respectively. Naked DNA and HC32-DATOU/MCC7 polyplex without heparin treatment were used as the controls. After 2 h incubation, all the samples were mixed with 10 μL PicoGreen working solution and incubated for another 5 minutes. Afterwards, the mixture solution was diluted by deionized water to a final concentration of 1 μg/mL in a black 96-well plate. Fluorescence measurements were carried out using a SpectraMax M3 plate reader with the excitation at 490 nm and the emission at 535 nm in quadruplicate. DNA release efficiency was quantified by normalizing the fluorescence intensity of samples to naked DNA control.

Size and Zeta Potential Measurements of Polyplexes

Polyplex size was measured by nanoparticle tracking analysis (NTA) using a Nanosight NS300. Polyplexes were prepared using 0.5 μg DNA with 30:1 w/w ratio in 10 μL SA. Next, the polyplex solution was diluted to 1 mL distilled water and then subjected to NTA analysis. A 60 second movie containing the Brownian motion tracking of the particles was recorded using the NTA software (Version 3.2). 10 tracks were assessed for each sample. Zeta potential measurements of polyplexes was conducted using a Malvern Instruments Zetasizer (Nano-ZS90) at a 90° scattering detector angle.

Transmission Electron Microscopy (TEM) Observation

Morphology of polyplexes was characterized by TEM. 80 μL polyplex solution with 2 μg MCC7 at the w/w ratio of 30:1 was centrifuged and the supernatant was discarded, and then polyplexes were further washed with 80 μL distilled water twice to remove excess salts. Afterwards, polyplexes were resuspended to a final volume of 10 μL distilled water. Then 2.5 μL polyplex solution was cast onto Formvar support films on 200 mesh copper grids and lyophilized immediately. Images were captured on a FEI Tecnai 120 TEM at 120 kV in UCD Conway Imaging Core Center.

Cell Culture

RDEBK and human keratinocyte (NHK) cells were cultured in keratinocyte cell basal medium (KBM-Gold) with the supplement pack (KGM-Gold SingleQuots) and 1% PS in a humid incubator with 5% CO2 at 37° C. under standard cell culture conditions.

GFP Expression and Cell Viability

GFP reporter gene transfection was first performed to evaluate the gene transfection efficiency of four HC32-DATOU polymers and screen out the best-performing candidate. RDEBKs were seeded in 96-well plates at a density of 2×10⁴ cells per well. Next day, 0.5 μg plasmid DNA encoding GFP was used for each well. HC32-DATOU polyplexes with different M_{w}s were prepared at polymer/DNA w/w ratios of 10:1, 30:1 and 50:1 in 20 μL SA, which was mixed with 80 μL fresh culture medium as the transfection medium. 4 h post transfection, transfection medium was replaced with fresh medium. 48 h post transfection, GFP expression of cells were visualized under a fluorescence microscope (Olympus IX81). Cell viability was measured with Alamarblue assay. Cell supernatants were removed and then cells were incubated with 10% Alamarblue reagent in HBSS for another 1 h at 37° C. Afterwards, the Alamarblue solution was transferred to a flat bottomed 96-well plate. Fluorescence intensity was read by a SpectraMax M3 plate reader with an excitation at 570 nm and emission at 590 nm. Fluorescence intensity of the untreated cell group was plotted as 100% viable. Cell viability was measured in quadruplicate and calculated by normalizing the fluorescence intensity of sample to that of the untreated group. HC32-DATOU showing the highest GFP expression and cell viability was used for the following studies.

In the formulation studies (see Example 10, below), SuperFect/DNA polyplexes were prepared at the w/w ratio of 3:1 according to the publication (Zeng, M.; Zhou, D.; Alshehri, F.; Lara-Sáez, I.; Lyu, Y.; Creagh-Flynn, J.; Xu, Q.; A, S.; Zhang, J.; Wang, W. Manipulation of Transgene Expression in Fibroblast Cells by a Multifunctional Linear-Branched Hybrid Poly(β-Amino Ester) Synthesized through an Oligomer Combination Approach. Nano Lett. 2019, 19 (1), 381-391. https://doi.org/10.1021/acs.nanolett.8b04098. Theoharis, S.; Krueger, U.; Tan, P. H.; Haskard, D. O.; Weber, M.; George, A. J. T. Targeting Gene Delivery to Activated Vascular Endothelium Using Anti E/P-Selectin Antibody Linked to PAMAM Dendrimers. J. Immunol. Methods 2009, 343 (2), 79-90. https://doi.org/10.1016/j.jim.2008.12.005.). Lipofectamine 2000/DNA lipoplexes were prepared according to manufacturer's protocol (2:1 volume/weight ratio). The median fluorescence intensity (MFI) and GFP-positive cells were quantified by flow cytometry on an Accuri C6 system in triplicate and further analyzed with Flowjo V10 software. 1×10⁴ cells were counted for each run.

Gluc Reporter Gene Transfection and Cell Viability

The HC32-DATOU was further evaluated in Gluc reporter gene transfection studies. Using 0.5 μg plasmid DNA encoding Gluc for each well, HC32-DATOU/DNA polyplexes were prepared at the w/w ratios of 20:1, 30:1 and 40:1, respectively. According to previous publications (Green, J. J.; Zugates, G. T.; Tedford, N. C.; Huang, Y.-H.; Griffith, L. G.; Lauffenburger, D. A.; Sawicki, J. A.; Langer, R.; Anderson, D. G. Combinatorial Modification of Degradable Polymers Enables Transfection of Human Cells Comparable to Adenovirus. Adv. Mater. 2007, 19 (19), 2836-2842. https://doi.org/10.1002/adma.200700371. Huang, J.-Y.; Gao, Y.; Cutlar, L.; O'Keeffe-Ahern, J.; Zhao, T.; Lin, F.-H.; Zhou, D.; McMahon, S.; Greiser, U.; Wang, W.; et al. Tailoring Highly Branched Poly(Beta-Amino Ester)s: A Synthetic Platform for Epidermal Gene Therapy. Chem. Commun. (Camb). 2015, 51 (40), 8473-8476. https://doi.org/10.1039/c5cc02193f. Zeng, M.; Zhou, D.; Ng, S.; Ahern, J. O.; Alshehri, F.; Gao, Y.; Pierucci, L.; Greiser, U.; Wang, W. Highly Branched Poly(5-Amino-1-Pentanol-Co-1,4-Butanediol Diacrylate) for High Performance Gene Transfection. Polymers (Basel). 2017, 9 (12), 161. https://doi.org/10.3390/polym9050161.), PEI/DNA polyplexes were prepared at w/w ratios of 1:1, 2:1 and 3:1, respectively.

RDEBKs were seeded and gene transfection was carried out as mentioned above. 48 h post transfection, to quantify the gene transfection efficiency, 50 μL of the cell supernatant was mixed with equal volume of Gluc assay working solution. Fluorescence intensity of the mixture was measured using a SpectraMax M3 plate reader with an excitation at 485 nm and emission at 525 nm. Gluc activity results were plotted in terms of relative light units (RLU). Cell viability were measured as mentioned above. Both Gluc activity and cell viability experiments were determined in quadruplicate.

Cellular Uptake of Polyplexes

Cy3 DNA labelling kits were used to label MCC7 according to standard protocol. RDEBKs were seeded in 96-well plates at a density of 1×10⁴ cells per well. Next day, using 0.25 μg MCC7 for each well, cells were transfected with HC32-DATOU/MCC7 polyplexes (w/w=30:1) and PEI/MCC7 (w/w=1:1) for 4 h, and then fixed with 4% PFA, permeabilized with 0.1% triton-100 and incubated with DAPI at a working concentration of 1 μg/mL in HBSS. Fluorescent images were taken with a microscope (Olympus IX81). The MFI and Cy3-positive proportion of cells were quantified by an Accuri C6 system in triplicate. Results were further analyzed with Flowjo V10 software with 1×10⁴ cells counted for each measurement.

Quantitative Reverse Transcription Polymerase Chain Reaction (RT-qPCR)

RT-qPCR was performed to quantify the COL7A1 mRNA expression. RDEBKs were seeded on 6-well plates at a density of 2.5×10⁵ cells per well one day prior to transfection. Cells were transfected with HC32-DATOU/MCC7 and PEI/MCC7 polyplexes complexed with 5 μg DNA at w/w ratios of 30:1 and 1:1. Three days post treatment, both treated and untreated cells were harvested and subjected to the purification of total RNA. RNA Extraction work was carried out according to the protocol of RNeasy Mini Kit. Next, 0.5 μg of total RNA from each group was used to synthesize the first-strand cDNA. The reverse transcription was performed with the primer 50 μM Oligo(dT)₂₀ according to the protocol of SuperScript III First-Strand Synthesis SuperMix. Afterwards, 1 μL of the final complementary DNA (cDNA) product was added to 9 μL of reaction mix (0.5 μL TaqMan primer, 5 μL TaqMan PCR mix, 3.5 μL RNase free water) which was loaded to one well of 384-well plates. Each sample was measured in triplicate. For COL7A1 quantitative gene expression, GAPDH was used as the endogenous control. Comparative CT values and TaqMan Reagents, QuantStudio 7 Flex System were set up for the experiments. Results were analyzed with the QuantStudio Real-Time PCR Software.

Cyto-Immunofluorescence Staining of C7

Cyto-immunofluorescence staining was used to determine C7 restoration of RDEBKs after treatment with HC32-DATOU/MCC7 and PEI/MCC7 polyplexes. 1.5×10⁴ cells were seeded on each coverslip in an 8-well chamber (Ibidi). 1 μg MCC7 was used for each well, HC32-DATOU/MCC7 and PEI/MCC7 polyplexes were prepared with the w/w ratio of 30:1 and 1:1, respectively. 3 days post transfection, cells were fixed with 4% PFA, permeated with 0.1% Triton X-100 and blocked in 5% goat serum in 1×DPBS for 1 h at RT, and then incubated with primary antibody (monoclonal anti-collagen, Type VII antibody produced in mouse) at 4° C. overnight at an antibody dilution of 1:200 in blocking buffer. Afterwards, cells were incubated with the secondary antibody (Alexa-568 goat anti-mouse IgG (H+L) at a dilution of 1:800 in blocking buffer). After final washes, the coverslips were mounted with Fluoroshield mounting medium with DAPI. Finally, cell images were captured with a fluorescence microscope (Olympus IX81).

Western Blotting

RDEBKs were seeded in a T-75 flask at a density of 1.5×10⁶ cells per flask one day prior to transfection. HC32-DATOU/MCC7 and PEI/MCC7 polyplexes at the w/w ratio of 30:1 and 1:1, respectively, were used for the transfection with 39 μg MCC7 for each flask. Four days post transfection, cells were harvested and treated with RIPA Lysis buffer which enables efficient cell lysis and solubilization of cellular proteins. 1 μL PIC was added to the cell lysis to a final volume of 50 μL and stored at −80° C. Bradford Assay was used to quantify the concentration of protein by normalizing the sample concentrations to the known BSA concentration. 40 μg denatured protein samples were loaded into the SDS-Page gel (4%-10%), and then electrophoresis was run at 75 V for 20 minutes followed by 120 V for 1 h. Protein samples were then transferred onto nitrocellulose membrane at 80 V for 1 h at RT followed by 90 V for 30 minutes at 4° C. Membrane blocking was carried out in the blocking buffer (5% BSA in TBST buffer) at RT for 1 h. β-Actin was used as the endogenous control. Then primary antibodies (polycolonal anti-C7 rabbit antibody and anti-actin mouse antibody at 2500 dilution in blocking buffer) were added to the membrane and incubated at 4° C. overnight. Following washing steps, secondary antibodies (anti-rabbit HRP and anti-mouse HRP at 5000 dilution in blocking buffer) were added to the membrane and incubated for 1 h at RT. After 3 times of TB ST washing, the membrane was visualized with the Pierce ECL Plus Substrate.

Statistics

SPSS Statistics for windows version 24 (IBM Corp., Armonk, N.Y., USA) was used for statistics. Student's t-test was used to analyze all the gene transfection data, which were expressed as mean±standard deviation (SD). For all analyses, p value <0.05 was considered statistically significant.

Results:

Reporter Gene Transfection in RDEBK Using HPAEs of Different Molecular Weights

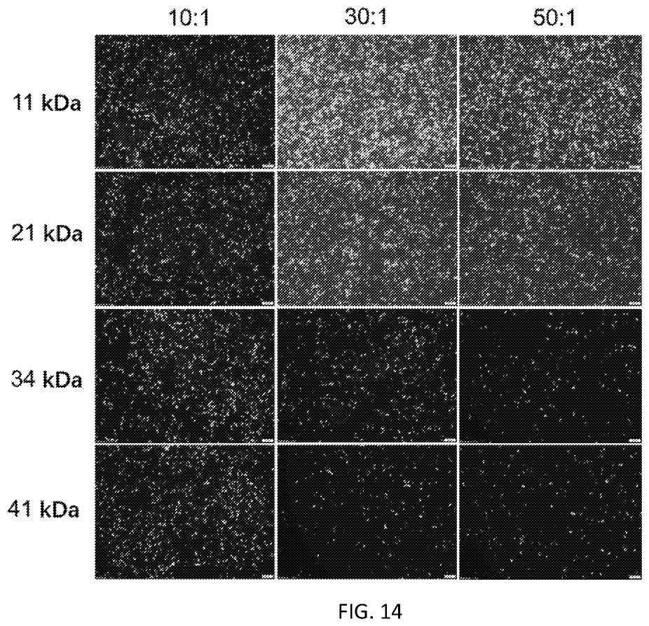

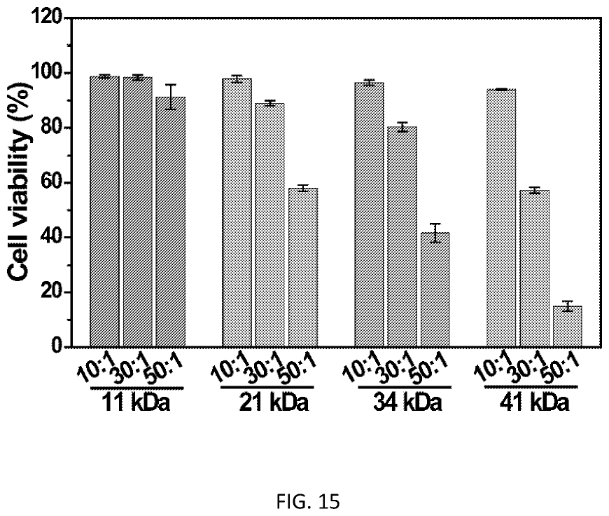

To identify the most favorable MW for gene delivery, four HC32-DATOU polymers with different M_{w}s were used to transfect RDEBK cells using GFP-encoding DNA as the reporter gene. As shown in FIG. 14 and FIG. 15, although no obvious cytotoxicity is observed at the polymer/DNA w/w ratio of 10:1, the transfection efficiency from all HC32-DATOU polymers is relatively low. When the w/w ratio is increased to 30:1 or greater, among all the polymers, 11 kDa HC32-DATOU at 30:1 achieves the highest transfection efficiency with the strongest GFP expression, while preserving high level cell viability of 98%. Generally, GFP expression decreased with the increasing M_{w} of HC32-DATOU polymers.

On the other hand, the cytotoxicity correlates very well with the increasing polymer M_{w}. For example, at the w/w of 50:1, as the M_{w} increases, cell viability decreases from 91% to 58%, 42% and 15%, respectively. Transfection efficiency is compromised with the increasing cytotoxicity which might be attributed to the incremental main chain of the polymer. These results demonstrate that M_{W} has significant effects on the transfection performance of HC32-DATOU, and a ˜10 kDa M_{w} is more favorable for RDEBK gene transfection to achieve both high transfection efficiency and low cytotoxicity.

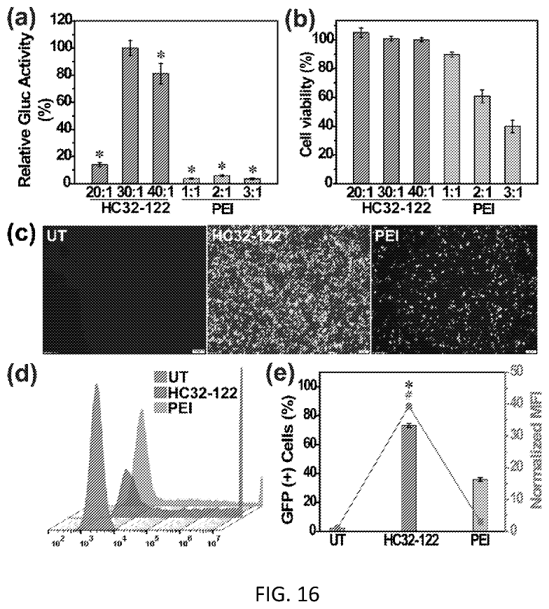

The high gene transfection ability of the 11 kDa HC32-DATOU was verified by comparing with the commercial gene transfection reagent PEI (M_{w}=25 kDa). As shown in FIG. 16a, at all three tested w/w ratios, the relative Gluc activity of RDEBK cells after transfection by HC32-DATOU/DNA polyplexes was much higher than that mediated by the PEI/DNA counterparts. The highest Gluc activity achieved by HC32-DATOU/DNA polyplexes at 30:1 w/w ratio was 17-fold higher than the PEI/DNA counterparts at the w/w ratio of 2:1.

Importantly, the HC32-DATOU/DNA polyplexes did not induce obvious cytotoxicity and preserved almost 100% cell viability. By contrast, PEI showed evident dose-independent cytotoxicity, the cell viability decreased significantly from 90% at 1:1 w/w ratio to 38% at 3:1 w/w ratio (FIG. 16b). Due to the considerable cytotoxicity, PEI with 1:1 w/w ratio was used for following studies.

To validate the high performance of HC32-DATOU/DNA polyplexes, GFP-encoding DNA was used for transfection. HC32-DATOU/DNA polyplexes mediated a much higher level of GFP expression than PEI, evidenced by the substantially stronger green fluorescence observed (FIG. 16c). Correspondingly, flow cytometry quantification analysis shows that more cells are shifted corresponding to the GFP-determining channel (FIG. 16d). 75% of the RDEBK cells were GFP-positive after transfection by the HC32-DATOU/DNA polyplexes, in contrast to 39% achieved by the PEI/DNA polyplexes. Moreover, the MFI of the RDEBK cells transfected by the HC32-DATOU/DNA polyplexes is 13-fold higher than that mediated by the PEI/DNA counterparts (FIG. 16e), indicating that much higher gene expression was achieved in individual cells. All these results demonstrate that HC32-DATOU is much more efficient and safer than PEI for gene transfection in RDEBK cells.

Biosynthesis of MCC7 and Cellular Uptake of HPAE/MCC7 Polyplexes by RDEBK Cells

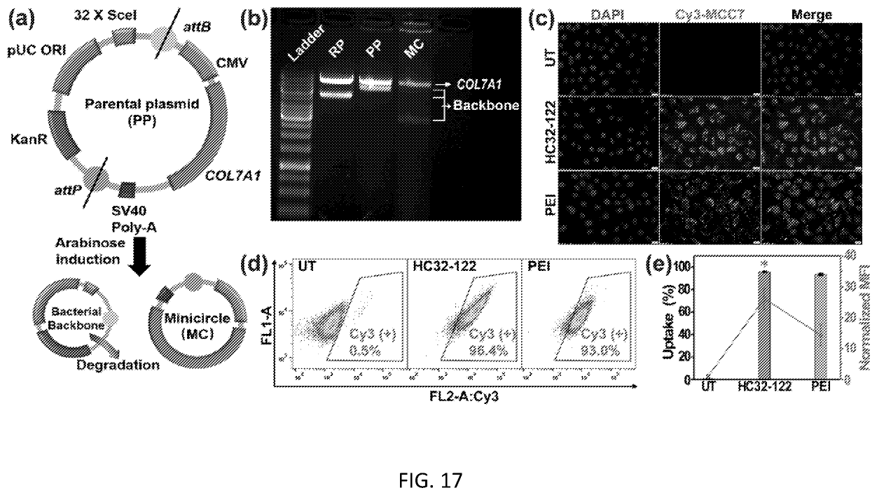

Utilizing a phiC31 plus 1-Scel digest system of minicircle technology (Gaspar, V.; de Melo-Diogo, D.; Costa, E.; Moreira, A.; Queiroz, J.; Pichon, C.; Correia, I.; Sousa, F. Minicircle DNA Vectors for Gene Therapy: Advances and Applications. Expert Opin. Biol. Ther. 2015, 15 (3), 353-379. https://doi.org/10.1517/14712598.2015.996544.), a minicircle DNA encoding the ˜9 kb full-length COL7A1 was biosynthesized (FIG. 17a). Gel electrophoresis shows that among all the three COL7A1-tagged DNA, MCC7 only has 3 kb length of backbone, which is 2 kb and 5 kb shorter than the regular plasmid (RP) pcDNA3.1COL7A1 and parental plasmid (PP) MN511A-1-COL7A1 (FIG. 17b), respectively, indicating the MCC7 with miniaturized derivative from the traditional PP vector devoid of bacterial sequences.

Cellular uptake of HC32-DATOU/MCC7 polyplexes was conducted in RDEBK cells. MCC7 was labelled with the red fluorescent dye Cy3, HC32-DATOU/MCC7 polyplexes were prepared as mentioned above. After 4 h of transfection, very strong red fluorescence was observed around the nucleus in the cells (FIG. 17c). In comparison, the PEI/MCC7 polyplexes show much lower cellular uptake efficiency, as evidenced by the much weaker red fluorescence. Flow cytometry measurements further demonstrate that although the percentage of Cy3-positive cells is similar (96.4% versus 93%, FIG. 17d), MFI of the cells incubated with the HC32-DATOU/MCC7 polyplexes was around 2-fold higher than that treated by the PEI/MCC7 counterparts (FIG. 17e), indicating a higher number of DNA copies was taken up by RDEBK cells. The maximum DNA sizes that RV and adeno-associated virus (AAV) vectors can carry are 7-8 kb and 5 kb, respectively. The fact that HC32-DATOU is capable of delivering 12 kb-length MCC7 into RDEBK cells in an efficient manner highlights its potential to achieve high C7 expression for RDEB treatment.

High Levels of COL7A1 mRNA and Recombinant C7 Expression

Following internalization, vector/DNA polyplexes are challenged by intra-cellular barriers, including endo/lysosomal escape, transport through cytoplasm, DNA release and nucleus entry. The cell cycle is another obstacle to nuclear uptake efficiency of cells undergoing mitosis is greater than 10 times higher than those in the growth phase of the cell cycle.

To evaluate the transcript COL7A1 mRNA and C7 protein expression mediated by the HC32-DATOU/MCC7 polyplexes, RT-qPCR, immunofluorescence staining and western blotting studies were performed.

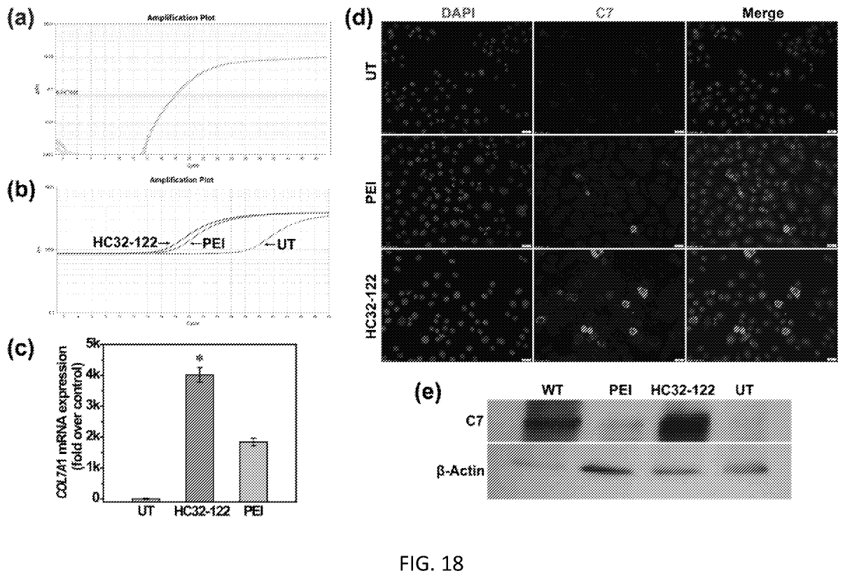

FIG. 18a and FIG. 18b outline the RT-qPCR amplification plots of the endogenous control GAPDH and COL7A1 mRNA expression, respectively. After normalized to the endogenous control, it is shown that the HC32-DATOU/MCC7 polyplexes mediated a 4019-fold upregulation of COL7A1 mRNA expression in comparison with the UT cells (FIG. 18c), 2.2-fold higher than that mediated by the PEI/MCC7 polyplexes. Immunofluorescence staining studies (FIG. 18d) further reveals that null-C7 expression was detected for the untreated RDEBK cells. In contrast, after transfection with the HC32-DATOU/MCC7 polyplexes, much higher level of cellular C7 expression around the nucleus was observed in the cyto-immunofluorescence images. Moreover, in agreement with the results of COL7A1 mRNA expression, HC32-DATOU/DNA polyplexes mediated more efficient C7 expression than the PEI/MCC7 counterparts.

Western blotting results show that no C7 secretion was detected from untreated RDEBK cells (FIG. 18e). On the contrary, a very clear 290-kDa protein band of C7 is visible after transfection with HC32-DATOU/polyplexes, the C7 band is even as strong as that of the wild-type NHK cells with full function of C7 production. It is noted that although PEI/MCC7 polyplexes achieved significant high level of COL7A1 mRNA expression, C7 production is limited.

A mechanism study demonstrated that the compact DNA structure increased cellular uptake, intracellular vector copy numbers, nuclear location and mRNA transcription levels (Kobelt, D.; Schleef, M.; Schmeer, M.; Aumann, J.; Schlag, P. M.; Walther, W. Performance of High Quality Minicircle DNA for in Vitro and in Vivo Gene Transfer. Mol. Biotechnol. 2013, 53 (1), 80-89. https://doi.org/10.1007/s12033-012-9535-6.). Herein, taking advantage of the optimized polymer and miniaturized gene construct, HC32-DATOU can effectively deliver COL7A1 gene into RDEBK cells, promote subsequent mRNA transcription and ultimate recombinant C7 expression, thereby strengthening the skin integrity.

Mechanistic Study of High Gene Transfection Efficiency of HPAE/MCC7 Polyplexes

DNA condensation, binding, polyplex size, zeta potential, morphology and DNA release are related to transfection performance. To better understand the mechanisms of the high gene transfection efficiency mediated by HC32-DATOU/MCC7 polyplexes, these physicochemical parameters were investigated.

The cationic HC32-DATOU is believed to condense the negatively charged MCC7 to form polyplexes via electrostatic self-assembly (FIG. 19a). To confirm this, agarose gel electrophoresis was conducted to evaluate the DNA condensation ability of HC32-DATOU 2 h post polyplex preparation. As shown in FIG. 19b, naked MCC7 DNA shifted on the gel, whereas HC32-DATOU was capable of condensing DNA on the well without obvious DNA shifting. Heparin competition assay further showed that HC32-DATOU Polyplexes with low heparin concentrations (0.1-0.3 IU/μL) still condensed the majority of DNA, suggesting strong DNA condensation ability and highly stable property of HC32-DATOU polyplexes.

Similarly, as quantified by PicoGreen Assay (FIG. 19c), HC32-DATOU showed stable and high DNA binding affinity of 96.3%, indicating only 3.7% of DNA unpacking. When 3 and 6 IU/μL concentrations of heparin were applied, 68.2% and 99.6% of DNA unpacking were detected respectively. These results demonstrate that HC32-DATOU condenses, binds and releases DNA efficiently in a controlled manner with the presence of adjustable negatively-charged heparin.

At the optimized w/w ratio for efficient C7 expression (30:1), nanoparticles exhibited a mean size of 110 nm and a mode size of 81 nm, respectively (FIG. 19d), with the zeta potential of +37.4 mV (FIG. 19e), indicating a compact nanoparticle structure with positive surface charge. It is known that polyplexes were most commonly formed of spherical or toroidal shape. Here, HC32-DATOU/MCC7 polyplexes manifested uniform and spherical morphology (FIG. 19f).

The transfection efficiency enhancement is reported to be benefited from the diamine end group modification, which increases the polymers' cationic charge, leading to improve the polymer/DNA binding dynamics and the condensation of DNA into nanoparticles and protect DNA from degradation. Apart from the end-modification with diamine DATOU, multiple DNA-binding/condensation moieties—including primary, secondary, tertiary amines—reside in HC32 backbone and terminal groups. Generally, LPAE/DNA particles are less than 250 nm and particles of smaller size were found to be more efficiently internalized by cells.

Without being bound by any theory, it is possible that the small, compact, uniform and cationic properties of HC32/MCC7 polyplexes enable the high cellular uptake efficiency. In addition, multiple ionizable secondary and tertiary amines can buffer a wide range of protons, which may facilitate the endo/lysosomal escape via the “proton sponge effect”. Afterwards, the stable gene packaging stability of HC32-DATOU suggests that it can assist the intracellular transport of polyplexes through the cytoplasm toward the nucleus.

An efficient vector must balance sufficient binding strength to protect DNA with the ability to release DNA. Without being bound by any theory, the moderate electrostatic interaction between HC32-DATOU and MCC7 and the biodegradable property of HC32-DATOU may facilitate gene release from polyplexes inside nucleus to start the transcription steps.

### Example 10: Lyophilized Compositions Comprising the HPAE Polyplexes of the Present Disclosure

The following example describes the results from varying the storage conditions and cryoprotectant concentration in the lyophilization process for exemplary HPAE/DNA polyplex formulations.

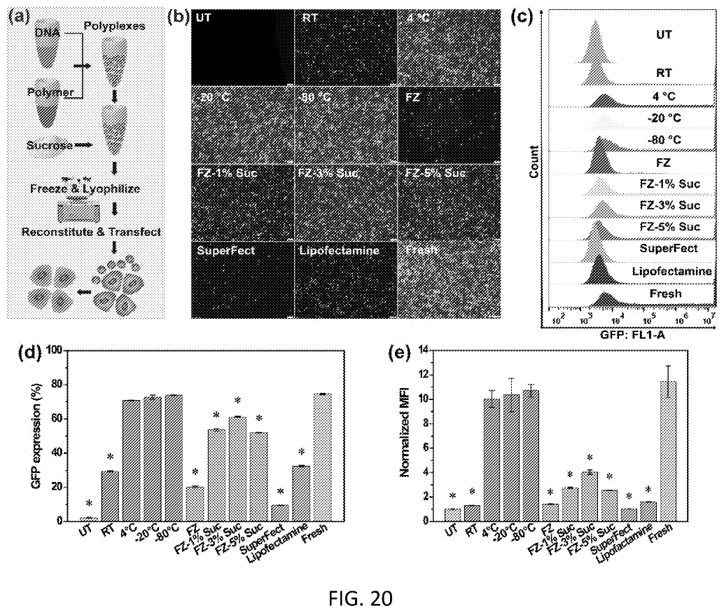

The HC32-DATOU/DNA polyplex formulation was optimized by varying the storage condition and cryoprotectant concentration in the lyophilization process. FIG. 20a illustrates the scheme of polyplex lyophilization fabrication and gene transfection studies in RDEBK cells using GFP-encoding DNA. Besides the fresh prepared polyplex, all polyplexes were employed in gene transfection 1 day post preparation. As shown in FIG. 20b, by varying the storage temperature, except the one stored at RT, fresh polyplexes and polyplexes stored at 4° C., −20° C. and −80° C. show comparable and high GFP expression with significant shift of the cell population in the flow cytometry histogram distributions (FIG. 20c). As shown in FIGS. 20d and 20e, the efficiency quantified with flow cytometry was higher than 70%, and the normalized MFI was around 10-fold higher than the UT group. These results demonstrate that low storage temperature is favorable for maintaining the high gene transfection ability of HC32-DATOU/DNA polyplexes.

Next, using sucrose as the cryoprotectant during freeze-drying process, the effects of cryoprotectant concentration on the gene transfection ability of HC32-DATOU/DNA polyplexes was studied. Freeze-drying polyplexes without any cryoprotectant (0% sucrose) resulted in the loss of the majority of transfection ability exhibiting 20% efficiency and only 1.4-fold higher MFI compared to the UT cells.

When 1%, 3% and 5% sucrose was added into the polyplex solution prior to lyophilization, transfection efficiency was increased to 54%, 61% and 52%, respectively. These results demonstrate that 3% of sucrose is more efficient for maintaining the gene transfection ability of HC32-DATOU/DNA polyplexes. Although the gene transfection was somewhat lower than freshly prepared counterparts, gene transfection ability of the polyplexes stored at low temperature or freeze-dried with sucrose is still much higher than that of the commercial reagents SuperFect and Lipofectamine, which is 10% and 32% efficiency, respectively.

Furthermore, polyplex lyophilization has unique advantages. First, it enables subsequent reconstruction of polyplexes at a higher concentration, which is particularly beneficial for in vivo injection that requires a limited administration volume. Second, easily adjustable solute (sucrose) can make the reconstructed polyplex solution isotonic during formulation. Also, lyophilized polyplexes with sucrose are expected to be more stable in the presence of serum compared with freshly prepared polyplexes. Finally, lyophilized polyplexes can be stored for years without losing efficacy.

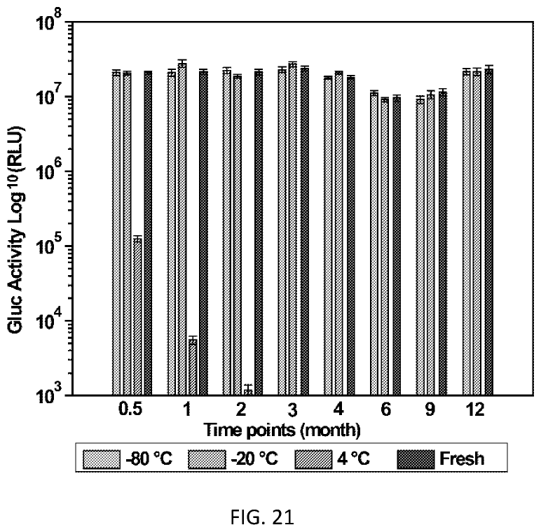

A gene transfection study of the top-performing HC32-DATOU/DNA polyplexes (4° C., −20° C. and −80° C. groups) with different storage time was carried out to evaluate shelf life. As shown in FIG. 21, after storage at 4° C. for 0.5 or 1 month, Gluc activity of the polyplexes was two to three magnitudes lower than that mediated by the freshly prepared ones. After 2 months, efficiency of the polyplexes became negligible.

In contrast, even after one year, polyplexes stored at −20° C. and −80° C. mediated the same level of Gluc activity as the freshly formulated polyplexes. These results demonstrate that the HC32-DATOU/DNA polyplexes are very stable and retain their full function of gene transfection by simply storing at −20° C. or −80° C., making them highly feasible for clinical applications.

Materials for Examples 9 and 10: Monomers 5-amino-1-pentanol (AP, 99%), trimethylolpropane triacrylate (TMPTA, 99%), 1,11-diamino-3,6,9-trioxaundecane (DATOU, 98%) were purchased from Sigma-Aldrich, and 1,4-butanediol diacrylate (BDA, 98%) was purchased from VWR. Chemicals lithium bromide (LiBr, 99%), tris-buffered saline and Tween 20 (TBST), paraformaldehyde (PFA) and triton X-100 were purchased from Sigma-Aldrich. Solvents dimethyl sulfoxide (DMSO, Sigma-Aldrich, 99%), dimethylformamide (DMF, Fisher Scientific, 99%), diethyl ether (Sigma-Aldrich, 99%) and deuterated chloroform (CDCl₃, Sigma-Aldrich, 99.9%) were used as received. Branched polyethyleneimine (PEI, M_{w}=25 kDa, Sigma-Aldrich), SuperFect (QIAGEN), Lipofectamine 2000 (Invitrogen) were used as the commercial reagent controls. Keratinocyte cell basal medium (Clonetics KBM-Gold) with the supplement pack (Clonetics KGM-Gold SingleQuots) was purchased from Lonza. Cell secreted Gaussia princeps luciferase (Glue) plasmid and BioLux Gaussia luciferase assay kits were obtained from New England Biolabs UK. Green fluorescent protein (GFP) plasmid was purchased from Aldevron. Hank's balanced salt solution (HBSS), sodium acetate (SA, pH 5.2±0.1, 3 M) buffer, tris acetate-EDTA (TAE) buffer and Radio-Immunoprecipitation assay (RIPA) buffer, agarose, bovine serum albumin (BSA), goat serum, monoclonal anti-C7 antibody produced in mouse, Protease Inhibitor Cocktail (PIC) and Bradford Reagent were purchased from Sigma-Aldrich. 1× Dulbecco's phosphate buffered saline (PBS), Gibco OPTI-MEM I reduced serum medium, 4′,6-diamidino-2-phenylindole (DAPI) and PicoGreen assay kits were purchased from Life Technologies. Penicillin-streptomycin (PS), EcoRI, Alexa-568 goat anti-mouse IgG (H+L) highly cross-adsorbed secondary antibody and Pierce ECL plus Western Blotting substrate were purchased from Thermo Fisher Scientific. Alamarblue assay kits, SYBR safe DNA gel stain and SuperScript III First-Strand Synthesis SuperMix were purchased from Invitrogen. Collagen type VII alpha 1 (Fam-MGB, primer & probe), human glyceraldehyde-3-phosphate dehydrogenase (GAPDH) endogenous control (VIC/MGB probe, primer limited) and TaqMan gene expression master mix were purchased from Applied Biosystems. TE buffer (QIAGEN), Cy3 DNA labelling kit (Mirus), RNeasy Mini Kit (QIAGEN), Fluoroshield mounting medium with DAPI (Abcam) were used as per manufacturers' protocols. Polyclonal anti-C7 rabbit primary antibody (Merck Millipore), anti-beta actin mouse primary antibody (Abcam), anti-rabbit IgG HRP-linked antibody (Cell Signaling) and anti-mouse IgG HRP-linked antibody (Cell Signaling) were used as received.

All documents cited or otherwise referenced or disclosed herein are incorporated by reference in their entirety for all purposes.

### REFERENCES

[1] L. Naldini, Gene therapy returns to centre stage. Nature 2015.
[2] H. Yin, R. L. Kanasty, A. A. Eltoukhy, A. J. Vegas, J. R. Dorkin, D. G. Anderson, Non-viral vectors for gene-based therapy. Nat. Rev. Genet. 2014.
[3] M. Foldvari, D. W. Chen, N. Nafissi, D. Calderon, L. Narsineni, A. Rafiee, J. Control. Release 2016.
[4] H. Lv, S. Zhang, B. Wang, S. Cui, J. Yan, Toxicity of cationic lipids and cationic polymers in gene delivery. J. Control. Release 2006.
[5] D. M. Lynn, R. Langer, J Am Chem Soc 2000.
[6] J. J. Green, G. T. Zugates, N. C. Tedford, Y. H. Huang, L. G. Griffith, D. A. Lauffenburger, J. A. Sawicki, R. Langer, D. G. Anderson, Adv. Mater. 2007.
[7] J. C. Sunshine, D. Y. Peng, J. J. Green, Mol. Pharm. 2012.
[8] D. G. Anderson, A. Akinc, N. Hossain, R. Langer, Mol. Ther. 2005.
[9] A. Akinc, D. M. Lynn, D. G. Anderson, R. Langer, J. Am. Chem. Soc. 2003.
[10] D. G. Anderson, D. M. Lynn, R. Langer, Angew. Chemie—Int. Ed. 2003.
[11] J. C. Sunshine, S. B. Sunshine, I. Bhutto, J. T. Handa, J. J. Green, PLoS One 2012.
[12] A. Mangraviti, S. Y. Tzeng, K. L. Kozielski, Y. Wang, Y. Jin, D. Gullotti, M. Pedone, N. Buaron, A. Liu, D. R. Wilson, S. K. Hansen, F. J. Rodriguez, G. D. Gao, F. Dimeco, H. Brem, A. Olivi, B. Tyler, J. J. Green, ACS Nano 2015.
[13] D. Zhou, L. Cutlar, Y. Gao, W. Wang, J. O. Keeffe-ahern, S. Mcmahon, B. Duarte, F. Larcher, B. J. Rodriguez, U. Greiser, W. Wang, Sci. Adv. 2016, 1.
[14] D. Zhou, Y. Gao, J. O'Keeffe Ahern, A. Sigen, Q. Xu, X. Huang, U. Greiser, W. Wang, ACS Appl. Mater. Interfaces 2016.
[15] D. Zhou, Y. Gao, A. Aied, L. Cutlar, O. Igoucheva, B. Newland, V. Alexeeve, U. Greiser, J. Uitto, W. Wang, J. Control. Release 2016.
[16] M. Zeng, D. Zhou, S. Ng, J. O. Ahern, F. Alshehri, Y. Gao, L. Pierucci, U. Greiser, W. Wang, Polymers (Basel). 2017, 9, 161.
[17] D. T. Woodley, M. Chen, Journal of Investigative Dermatology. 2015.
[18] J.-D. Fine, L. Bruckner-Tuderman, R. A. J. Eady, E. A. Bauer, J. W. Bauer, C. Has, A. Heagerty, H. Hintner, A. Hovnanian, M. F. Jonkman, I. Leigh, M. P. Marinkovich, A. E. Martinez, J. A. McGrath, J. E. Mellerio, C. Moss, D. F. Murrell, H. Shimizu, J. Uitto, D. Woodley, G. Zambruno, J. Am. Acad. Dermatol. 2014.
[19] L. Cutlar, D. Zhou, X. Hu, B. Duarte, U. Greiser, F. Larcher, W. Wang, A non-viral gene therapy for treatment of recessive dystrophic epidermolysis bullosa. Exp. Dermatol. 2016.
[20] J. W. Fountain, W. K. Lockwood, F. S. Collins, Gene 1988.
[21] T. Wong, L. Gammon, L. Liu, J. E. Mellerio, P. J. C. Dopping-Hepenstal, J. Pacy, G. Elia, R. Jeffery, I. M. Leigh, H. Naysaria, J. a McGrath, J. Invest. Dermatol. 2008.
[22] G. Petrof, M. Martinez-Queipo, J. E. Mellerio, P. Kemp, J. A. McGrath, Br. J. Dermatol. 2013.
[23] A. Nakayama, M. Sato, M. Shinohara, S. Matsubara, T. Yokomine, E. Akasaka, M. Yoshida, S. Takao, Cloning Stem Cells 2007.
[24] M. Lee, K. Chea, R. Pyda, M. Chua, I. Dominguez, J. Biomol. Tech. 2017.
[25] M. S. Tabar, M. Hesaraki, F. Esfandiari, F. S. Samani, H. Vakilian, H. Baharvand, Cell J. 2015.
[26] E. T. Jordan, M. Collins, J. Terefe, L. Ugozzoli, T. Rubio, J. Biomol. Tech. 2008.
[27] Y. Liu, D. Wu, Y. Ma, G. Tang, S. Wang, C. He, T. Chung, S. Goh, Chem. Commun. (Camb). 2003.
[28] J. J. Green, R. Langer, D. G. Anderson, A combinatorial polymer library approach yields insight into nonviral gene delivery. Acc. Chem. Res. 2008.
[29] D. W. Pack, A. S. Hoffman, S. Pun, P. S. Stayton, Design and development of polymers for gene delivery. Nat. Rev. Drug Discov. 2005.
[30] M. A. Mintzer, E. E. Simanek, Nonviral vectors for gene delivery. Chem. Rev. 2009.
[31] D. V. Schaffer, N. A. Fidelman, N. Dan, D. A. Lauffenburger, Biotechnol. Bioeng. 2000.
[32] O. Zelphati, F. C. Szoka, Proc. Natl. Acad. Sci. U.S.A. 1996.
[33] J. REJMAN, V. OBERLE, I. S. ZUHORN, D. HOEKSTRA, Biochem. J. 2004.
[34] A. Verma, O. Uzun, Y. Hu, Y. Hu, H. S. Han, N. Watson, S. Chen, D. J. Irvine, F. Stellacci, Nat. Mater. 2008.
[35] J. A. McGrath, A. Ishida-Yamamoto, A. O'Grady, I. M. Leigh, R. A. J. Eady, J. Invest. Dermatol. 1993, 100, 366.
[36] M. Goto, D. Sawamura, K. Ito, M. Abe, W. Nishie, K. Sakai, A. Shibaki, M. Akiyama, H. Shimizu, J. Invest. Dermatol. 2006.
[37] Z. Y. Chen, C. Y. He, A. Ehrhardt, M. A. Kay, Mol. Ther. 2003.
[38] F. Jia, K. D. Wilson, N. Sun, D. M. Gupta, M. Huang, Z. Li, N. J. Panetta, Z. Y. Chen, R. C. Robbins, M. A. Kay, M. T. Longaker, J. C. Wu, Nat. Methods 2010.
[39] C. Perdoni, M. J. Osborn, J. Tolar, Transl. Res. 2016.
[40] C. Georgiadis, F. Syed, A. Petrova, A. Abdul-Wahab, S. M. Lwin, F. Farzaneh, L. Chan, S. Ghani, R. A. Fleck, L. Glover, J. R. McMillan, M. Chen, A. J. Thrasher, J. A. McGrath, W. L. Di, W. Qasim, J. Invest. Dermatol. 2016.

1-141. (canceled)

142. A polyplex comprising a nucleic acid component and a highly branched poly(β-amino ester), wherein the nucleic acid component is a nanoplasmid, minicircle, or gene editing system, and the highly branched poly(β-amino ester) comprises formula (I): 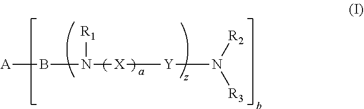
wherein each A is independently a linear or branched carbon chain of 1 to 30 carbon atoms, a linear or branched heteroatom-containing carbon chains of 1 to 30 atoms, a carbocycle containing 3 to 30 carbon atoms, or a heterocycle containing 3 to 30 atoms;
wherein A is optionally substituted with one or more halogen, hydroxyl, amino group, sulfonyl group, sulphonamide group, thiol, C₁-C₆ alkyl, C₁-C₆ alkoxy, C₁-C₆ether, C₁-C₆ thioether, C₁-C₆ sulfone, C₁-C₆ sulfoxide, C₁-C₆primary amide, C₁-C₆ secondary amide, halo C₁-C₆ alkyl, carboxyl group, cyano group, nitro group, nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂—C₅ heteroaryl or C₆-C₁₀ aryl; wherein each R′ is independently selected, from the group consisting of hydrogen and C₁-C₆ alkyl;
each B is independently 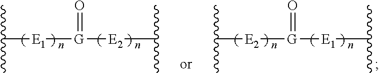
G is —C—, —S—, —S(O)—, —P(OR₁)—, or —P(OH)—; n is at least 1;
each E₁ is selected from the group consisting of covalent bond, —N—, —O—, —S—, alkylene, heteroalkylene, alkenyl, heteroalkenylene, alkynyl, heteroalkynylene;
each E₂ is selected from the group consisting of covalent bond, —N—, —O—, —S—, alkylene, heteroalkylene, alkenyl, heteroalkenylene, alkynyl, heteroalkynylene
each X is independently 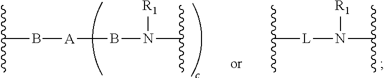
each Y is independently 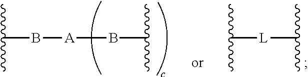
each L is independently a second linking moiety;
each R₁, R₂ and R₃ are independently, at each occurrence H, C₁-C₄₀alkyl, C₁-C₄₀ heteroalkyl, C₂-C₄₀alkenyl, C₂-C₄₀ heteroalkenylene, C₄-C₈cycloalkenyl, C₂-C₄₀alkynyl, C₂-C₄₀ heteroalkynylene, C₃-C₈cycloalkyl, heterocyclyl, aryl, or heteroaryl; wherein the heterocyclyl and heteroaryl contain 1-5 heteroatoms selected from the group consisting of N, S, P and O; wherein the C₁-C₆alkyl, C₂-C₈alkenyl, C₄-C₈cycloalkenyl, C₂-C₆alkynyl, C₃-C₈cycloalkyl, heterocyclyl, aryl, and heteroaryl are optionally substituted with D, halogen, C₁-C₆alkyl, —OH, —NH(C₁-C₆alkyl), or —N(C₁-C₆alkyl)₂; or
wherein R₂ and R₃ together with the atom to which they are attached can form heterocyclyl or heteroaryl containing 1-3 heteroatoms selected from the group consisting of N, S, P and O;
a is 1-1000;
b is 3 or 4;
c is 1-3; and
z is 1-100;
with the proviso that at least one of R₂ and R₃ is not H.

143. The polyplex of claim 142, wherein the nucleic acid component is a gene editing system.

144. The polyplex of claim 143, wherein the gene editing system is a (i) clustered, regularly interspaced, palindromic repeats (CRISPR)-associated (Cas) system; (ii) a transcription activator-like effector nuclease (TALEN) system; or (iii) a zinc finger nuclease (ZFN) system.

145. The polyplex of claim 144, wherein the gene editing system is a (i) clustered, regularly interspaced, palindromic repeats (CRISPR)-associated (Cas) system.

146. The polyplex of claim 145, wherein the gene is COL7A1.

147. The polyplex of claim 142, wherein the polyplex delivers a functional COL7A1 gene to promote the expression of type VII collagen protein (C7).

148. The polyplex of claim 142, wherein the polymer has a MW of between about 5 kDa and about 50 kDa.

149. The polyplex of claim 142, wherein the polymer has a MW of about 5 kDa to about 15 kDa.

150. The polyplex of claim 142, wherein the polymer has an alpha parameter defined from the Mark-Houwink of less than about 0.5.

151. The polyplex of claim 142, wherein the polymer has an alpha parameter defined from the Mark-Houwink equation ranging from about 0.3 to about 0.5.

152. A polyplex comprising a nucleic acid component and a highly branched poly(β-amino ester), wherein the nucleic acid component is a nanoplasmid, minicircle, or gene editing system, and the highly branched poly(β-amino ester) is made by a process comprising reacting together:
(a) a compound of formula (A) 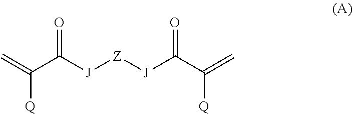
(b) a first amine having the formula R₁—NH₂ or R₁—N(H)—Z′—N(H)—R₁;
(c) a second amine having the formula R₂—NH₂ or R₂—N(H)—Z″—N(H)—R₂; and
(d) a compound of formula (B): 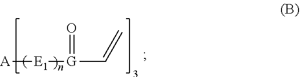
wherein
each J is independently —O— or —NH—;
Z, Z′, and Z′ are linking moieties;
A is an optionally substituted linear or branched carbon chain of 1 to 30 carbon atoms, an optionally substituted linear or branched heteroatom-containing carbon chains of 2 to 30 atoms, a carbocycle containing 3 to 30 carbon atoms, or an optionally substituted heterocycle containing 3 to 30 atoms;
G is —C—, —S—, —S(O)—, —P(OR₁)—, or —P(OH)—;
each Q is H or a C₁-C₁₀ linear or branched alkyl group;
each E₁ is independently selected from the group consisting of covalent bond, —N—, —O—, —S—, alkylene, heteroalkylene, alkenyl, heteroalkenylene, alkynyl, heteroalkynylene;
R₁ and R₂ are each independently C₁-C₄₀alkyl, C₁-C₄₀ heteroalkyl, C₂-C₄₀alkenyl, C₂-C₄₀ heteroalkenylene, C₄-C₈cycloalkenyl, C₂-C₄₀alkynyl, C₂-C₄₀ heteroalkynylene, C₃-C₈cycloalkyl, heterocyclyl, aryl, or heteroaryl; wherein the heterocyclyl and heteroaryl contain 1-5 heteroatoms selected from the group consisting of N, S, P and O; wherein the C₁-C₄₀alkyl, C₂-C₄₀alkenyl, C₄-C₈cycloalkenyl, C₂-C₄₀alkynyl, C₃-C₈cycloalkyl, heterocyclyl, aryl, and heteroaryl are optionally substituted with D, halogen, C₁-C₆alkyl, —OH, —O—C₁-C₆alkyl, —NH₂, —NH(C₁-C₆alkyl), or —N(C₁-C₆alkyl)₂; and R₁ is unsubstituted or substituted with at least one of a halogen, a hydroxyl, an amino group, a sulfonyl group, a sulphonamide group, a thiol, a C₁-C₆ alkyl, a C₁-C₆alkoxy, a C₁-C₆ ether, a C₁-C₆ thioether, a C₁-C₆ sulfone, a C₁-C₆ sulfoxide, a C₁-C₆ primary amide, a C₁-C₆ secondary amide, a halo C₁-C₆ alkyl, a carboxyl group, a cyano group, a nitro group, a nitroso group, —OC(O)NR′R′, —N(R′)C(O)NR′R′, —N(R′)C(O)O—C₁-C₆ alkyl, C₃-C₆cycloalkyl, C₃-C₆ heterocyclyl, C₂-C₅ heteroaryl and C₆-C₁₀) aryl; wherein each R′ is independently selected, from the group consisting of hydrogen and C₁-C₆ alkyl; and
each n is at least 1.

153. The polyplex of claim 152, wherein the compound of formula (B) is 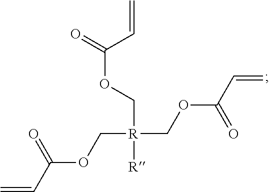
wherein
R is a linear or branched carbon chain of 1 to 10 carbon atoms, a linear or branched heteroatom-containing carbon chains of 1 to 10 atoms, a carbocycle containing 3 to 10 carbon atoms, or a heterocycle containing 3 to 10 atoms, and R is unsubstituted or substituted with at least one of a halogen, a hydroxyl, an amino group, a sulfonyl group, a sulphonamide group, a thiol, a C₁-C₆ alkyl, a C₁-C₆ alkoxy, a C₁-C₆ ether, a C₁-C₆ thioether, a C₁-C₆ sulfone, a C₁-C₆ sulfoxide, a C₁-C₆ primary amide, a C₁-C₆ secondary amide, a halo C₁-C₆alkyl, a carboxyl group, a cyano group, a nitro group, a nitroso group, —OC(O)NR′R′, —N(R′)C(O)NR′R′, N(R′)C(O)O—C₁-C₆alkyl, C₃-C₆ cycloalkyl, C₃-C₆ heterocyclyl, C₂-C₅ heteroaryl and C₆-C₁₀ aryl; wherein each R′ is independently selected, from the group consisting of hydrogen and C₁-C₆ alkyl; and
R″ is an unsubstituted or substituted, linear or branched carbon chain of 1 to 10 carbon atoms, a linear or branched heteroatom-containing carbon chains of 1 to 10 atoms, a carbocycle containing 3 to 10 carbon atoms, or a heterocycle containing 3 to 10 atoms.

154. The polyplex of claim 153, wherein:
a) J is O, each Q is H, and Z is 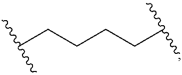
b) the first amine is R₁—NH₂, wherein R₁ is 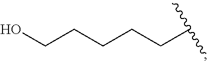
and
c) the second amine is R₂—NH₂, wherein R₂ is C₁-C₄₀alkyl wherein the C₁-C₄₀alkyl is optionally substituted with —NH₂.

155. The polyplex of claim 152, wherein:
a) J is O, each Q is H, and Z is 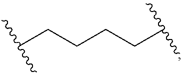
b) the first amine is R₁—NH₂, wherein R₁ is 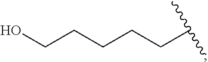
and
c) the second amine is R₂—NH₂, wherein R₂ is

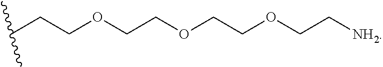 [SMILES: *CCOCCOCCOCCN]

156. The polyplex of claim 152, wherein the compound of formula (B) is

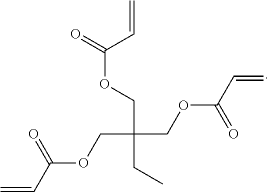

157. The polyplex of claim 142, wherein the polymer comprises: 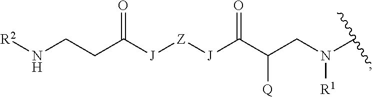
wherein J is O, each Q is H, and Z is 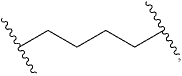
R₁ is 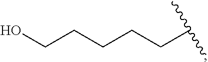
and R₂ is C₁-C₄₀alkyl wherein the C₁-C₄₀alkyl is optionally substituted with —NH₂ or

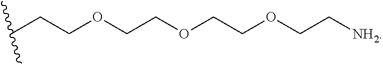 [SMILES: *CCOCCOCCOCCN]

158. The polyplex of claim 157, wherein the polymer comprises:

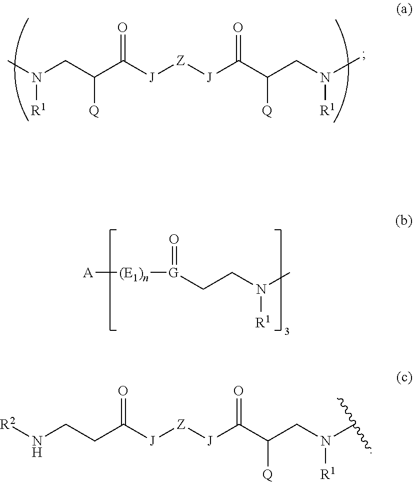

159. The polyplex of claim 157, wherein the polymer comprises:

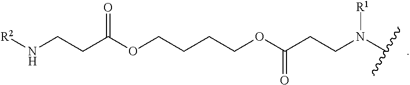

160. The polyplex of any of claim 142, wherein the polymer comprises: 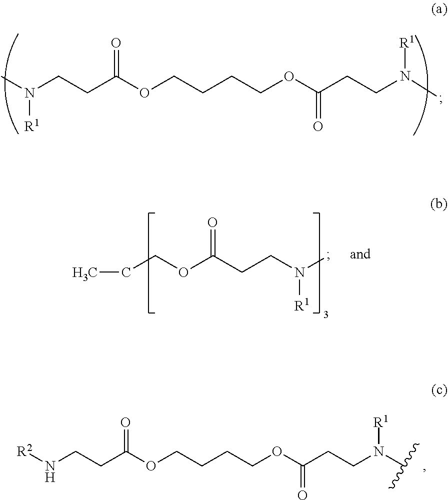
wherein
R₁ is 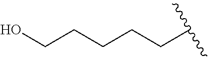 [SMILES: *CCCCCO]
and
R₂ is selected from

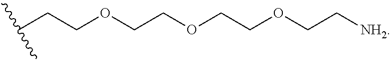 [SMILES: *CCOCCOCCOCCN]

161. The polyplex of any of claim 142, wherein the polymer comprises: 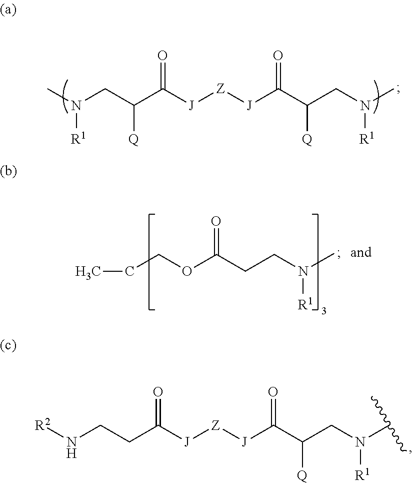
wherein
J is O and Z is 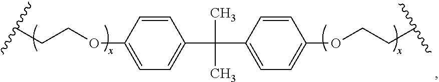
wherein x is 1-1000;
R₁ is 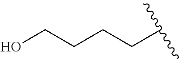 [SMILES: *CCCCO]
and
R₂ is

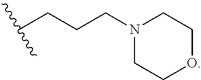 [SMILES: *CCCN1CCOCC1]

162. A pharmaceutical composition comprising an effective amount of a polyplex of claim 142, in combination with a pharmaceutically acceptable carrier.

163. A method of cell transfection comprising contacting one or more target cells with a pharmaceutical composition of claim 162 under conditions suitable to transfect the target cell with a polyplex.

164. A method of treating a disease in a patient in need thereof, comprising administering a therapeutically effective pharmaceutical composition of claim 162, wherein the administration of the composition corrects a defective translation of a target gene in the subject.

165. The method of claim 164, wherein the gene is COL7A1 and the disease is a form of epidermolysis bullosa.

166. The polyplex of claim 142, wherein the polymer is made by a process comprising reacting together:
(a) a compound of Formula (A), a compound of Formula (B), and a first amine (C), wherein:
(A) the compound of Formula (A) is: 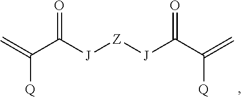
wherein J is O, each Q is H, and Z is 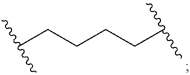 [SMILES: *CCCC*]
(B) the compound of Formula (B) is: 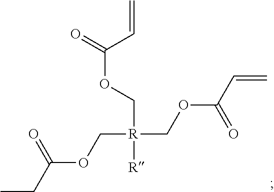
wherein
R is a carbon atom; and
R″ is an unsubstituted or substituted, linear or branched carbon chain of 1 to 10 carbon atoms, a linear or branched heteroatom-containing carbon chains of 1 to 10 atoms, a carbocycle containing 3 to 10 carbon atoms, or a heterocycle containing 3 to 10 atoms; and
(C) the first amine has the formula R₁—NH₂; wherein R₁ is 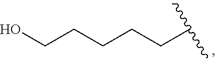 [SMILES: *CCCCCO]
and
(b) reacting the product of Step (a) with a second amine having the formula R₂—NH₂, wherein R₂ is C₁-C₄₀alkyl wherein the C₁-C₄₀alkyl is optionally substituted with —NH₂ or

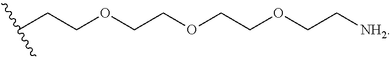 [SMILES: *CCOCCOCCOCCN]

167. The polyplex of claim 165, wherein R₂ is

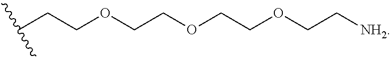 [SMILES: *CCOCCOCCOCCN]

168. A polyplex comprising a nucleic acid component and a highly branched poly(β-amino ester), wherein the nucleic acid component is a gene editing system, and wherein the highly branched poly(β-amino ester) is made by a process comprising reacting together:
(a) a compound of Formula (A), a compound of Formula (B), and a first amine (C), wherein:
(A) the compound of Formula (A) is bisphenol ethoxylate diacrylate;
(B) the compound of Formula (B) is trimethylolpropane triacrylate;
(C) the first amine (C) is 4-amino-1-butanol; and
(b) reacting the product of Step (a) with a second amine, wherein the second amine is 4-amino-1-butanol.

169. The polyplex of claim 168, wherein the gene editing system is a clustered, regularly interspaced, palindromic repeats (CRISPR)-associated (Cas) system.

170. The polyplex of claim 168, wherein the polyplex edits a target gene when administered to a subject in need thereof.

171. The polyplex of claim 170, wherein the target gene COL7A1.

172. The polyplex of claim 169, wherein the polyplex provides a functional COL7A1 gene to promote the expression of type VII collagen protein (C7) when administered to a subject in need thereof.

173. The polyplex of claim 168, wherein the polymer has a MW of between about 5 kDa and about 50 kDa.

174. The polyplex of claim 168, wherein the polymer has a MW of about 5 kDa to about 15 kDa.

175. The polyplex of claim 142, wherein the nucleic acid component comprises a gene associated with a genetic disease or disorder.

176. The polyplex of claim 175, wherein the genetic disease or disorder is caused by a mutation in one or more genes that results in low, absent, or dysfunctional protein expression.

177. The polyplex of claim 175, wherein the genetic disease or disorder is a genodermatosis disease.

178. The polyplex of claim 176, wherein the gene is selected from the group consisting of COL7A1, LAMB3, ADA, SERPINA1, CFTR, HTT, NF1, PHA, HBS, FERMT1, KRT14, DSP, SPINK5, and FLG.

179. The polyplex of claim 145, wherein the gene is associated with a genetic disease or disorder.

180. The polyplex of claim 179, wherein the genetic disease or disorder is caused by a mutation in one or more genes that results in low, absent, or dysfunctional protein expression.

181. The polyplex of claim 179, wherein the genetic disease or disorder is a genodermatosis disease.

182. The polyplex of claim 180, wherein the gene is selected from the group consisting of COL7A1, LAMB3, ADA, SERPINA1, CFTR, HTT, NF1, PHA, HBS, FERMT1, KRT14, DSP, SPINK5, and FLG.

183. The method of claim 164, wherein the disease is adenosine deaminase (ADA) deficiency, Alpha-1 Antitrypsin Deficiency, cystic fibrosis, Huntington's Disease, Neurofibromatosis Type 1, Phenylketonuria, Sickle Cell Disease, Sporadic Inclusion Body Myositis, Duchenne muscular dystrophy, Kindler syndrome, Junctional Epidermolysis Bullosa, Epidermolysis bullosa dystrophica (autosomal recessive), Epidermolysis bullosa dystrophica (localisata variant), Epidermolysis bullosa pruriginosa, Epidermolysis bullosa (pretibial), Dermatopathia pigmentosa reticularis, Epidermolysis bullosa simplex (Dowling-Meara-type), Epidermolysis bullosa simplex (Koebner-type), Epidermolysis bullosa simplex (recessive 1), Epidermolysis bullosa simplex (Weber-Cockayne-type), Naegeli-Franceschetti-Jadassohn syndrome, Epidermolysis bullosa (lethal acantholytic), Netherton Syndrome, Ichthyosis Vulgaris, Atopic Dermatitis, Usher's syndrome, Ehlers-Danlos syndrome, Homozygous Familial Hypercholesterolemia (HoFH), or Crohn's disease.
